(19) United States
(12) Patent Application Publication
(10) Pub. No.: US 2024/0341178 A1
(43) Pub. Date: Oct. 10, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hee-Ryong KANG, Gyeonggi-do (KR); So-Young JUNG, Gyeonggi-do (KR); Hyo-Soon PARK, Gyeonggi-do (KR); Jeong-Eun YANG, Gyeonggi-do (KR); Sang-Hee CHO, Gyeonggi-do (KR); So-Mi PARK, Gyeonggi-do (KR)

(21) Appl. No.: 18/592,645

(22) Filed: Mar. 1, 2024

(30) Foreign Application Priority Data
Apr. 6, 2023 (KR) 10-2023-0045313

(51) Int. Cl. H10K 85/60; C07D 413/04; C07D 413/14; C09K 11/06; H10K 50/11; H10K 101/00

(52) U.S. Cl. CPC H10K 85/657; C07D 413/04; C07D 413/14; C09K 11/06; H10K 85/615; H10K 85/636; H10K 85/654; H10K 85/6574; C09K 2211/1018; H10K 50/11; H10K 2101/90

(57) ABSTRACT

The present disclosure relates to a plurality of host materials, an organic electroluminescent compound, and an organic electroluminescent device comprising the same. By comprising a plurality of host materials and/or an organic electroluminescent compound according to the present disclosure, an organic electroluminescent device having low driving voltage and/or high luminous efficiency and/or long lifespan characteristics can be provided.

### TECHNICAL FIELD

The present disclosure relates to a plurality of host materials, an organic electroluminescent compound, and an organic electroluminescent device comprising the same.

### BACKGROUND ART

The TPD/Alq₃ bilayer small molecule organic electroluminescent device (OLED) with green-emission, which is constituted with a light-emitting layer and a charge transport layer, was first developed by Tang, et al., of Eastman Kodak in 1987. Thereafter, the studies on an OLED have been rapidly affected and OLEDs have been commercialized.

At present, an organic electroluminescent device mainly includes phosphorescent materials having excellent luminous efficiency in panel realization. An OLED having low driving voltage, high luminous efficiency and/or long lifespan characteristics is required for long time use and a high resolution of a display.

Korean Patent No. 1777454 and Chinese Patent Application Laid-open No. 110734431 disclose an OLED device comprising naphthoxazole-based compounds and naphthothiazole-based compounds as materials for a light-emitting layer, etc. However, said references do not specifically disclose a compound having an organic electroluminescent compound claimed in the present disclosure, as its basic skeleton, and also do not disclose a plurality of host materials comprising the same.

### DISCLOSURE OF THE INVENTION

### Problems to be Solved

The object of the present disclosure is firstly to provide an organic electroluminescent compound and a plurality of host materials which are able to produce an organic electroluminescent device with low driving voltage and/or high luminous efficiency and/or long lifespan characteristics; and secondly to provide an organic electroluminescent device comprising an organic electroluminescent compound and/or a plurality of host materials.

### Solution to Problems

As a result of intensive study to solve the above-detailed technical problem, the present inventors found that the aforementioned objective can be achieved by a plurality of host materials comprising at least one first host compound(s) represented by the following Formula 1 and at least one second host compound(s) represented by the following Formula 2, so that the present invention was completed.

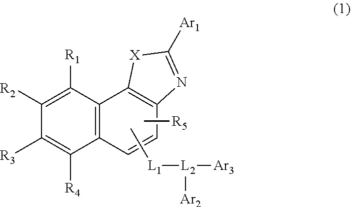

In Formula 1,
X represents —O— or —S—;
R₁ to R₅ each independently represent, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to 30-membered)heteroaryl, a substituted or unsubstituted (C1-C30)alkoxy, or —NRₐR_{b};
Rₐ and R_{b} each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
L₁ represents a single bond or a substituted or unsubstituted (C6-C30)arylene;
L₂ represents a substituted or unsubstituted nitrogen-containing (3- to 30-membered)heteroarylene; and
Ar₁ to Ar₃ each independently represent, a substituted or unsubstituted (C6-C30)aryl or a substituted or unsubstituted (3- to 30-membered)heteroaryl; provided that Ar₂ and Ar₃ are different from each other;

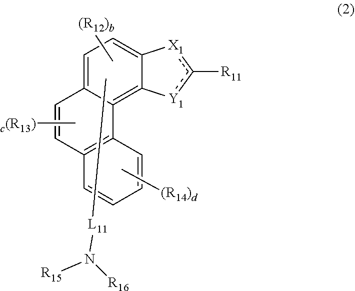

In Formula 2,
X₁ and Y₁ each independently represent, —N═, —NR_{c}, —O— or —S—, provided that any one of X₁ and Y₁ is —N═, and the other of X₁ and Y₁ is —NR_{c}—, —O— or —S—;
R₁₁ represents a substituted or unsubstituted (C6-C30)aryl, or a substituted or unsubstituted (3- to 30-membered)heteroaryl;
R₁₂ to R₁₄, and R_{c} each independently represent, hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -L₁₂-N(Ar₁₁)(Ar₁₂); or may be linked to the adjacent substituents to form a ring(s);
R₁₅ and R₁₆ each independently represent, a substituted or unsubstituted (C6-C30)aryl, or a substituted or unsubstituted (3- to 30-membered)heteroaryl;
L₁₁ and L₁₂ each independently represent, a single bond, a substituted or unsubstituted (C6-C30)arylene, or a substituted or unsubstituted (3- to 30-membered)heteroarylene;
Ar₁₁ and Ar₁₂ each independently represent, hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (3- to- 30-membered)heteroaryl; and
b and c each independently represent, an integer of 1 or 2, and d represents an integer of 1 to 4, and when b to d are an integer of 2 or more, each of R₁₂ to R₁₄ may be the same or different.

### Advantageous Effects of Invention

By using a plurality of host materials and/or an organic electroluminescent compound according to the present disclosure, an organic electroluminescent device having low driving voltage and/or high luminous efficiency and/or long lifespan characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound(s) represented by Formula 1 and at least one a second host compound(s) represented by Formula 2, and an organic electroluminescent device comprising the same.

The present disclosure relates to an organic electroluminescent compound represented by Formula 1, an organic electroluminescent material comprising the organic electroluminescent compound, and an organic electroluminescent device comprising the organic electroluminescent compound and/or the organic electroluminescent material.

The present disclosure relates to an organic electroluminescent compound represented by Formula 1, a plurality of host materials comprising the organic electroluminescent compound, and an organic electroluminescent device comprising the organic electroluminescent compound and/or the plurality of host materials.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc. The hole transport band material may be one or more selected from the group consisting of a hole transport material, a hole injection material, an electron blocking material, a hole auxiliary material, and a light-emitting auxiliary material.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C2-C30)alkenyl” is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. Herein, the term “(C2-C30)alkynyl” is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms, preferably 5 to 7 ring backbone atoms and at least one heteroatom selected from the group consisting of B, N, O, S, Si, and P, preferably O, S, and N, and includes tetrahydrofuran, pyrrolidine, thiolan, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The term “(3- to 30-membered)heteroaryl(ene)” in the present disclosure is an aryl having 3 to 30 ring backbone atoms including at least one heteroatom selected from the group consisting of B, N, O, S, Si, P, Se, and Ge in which the number of the ring backbone carbon atoms is preferably 5 to 25. The number of the heteroatoms in the heteroaryl is preferably 1 to 4. The above heteroaryl may be a monocyclic ring, or a fused ring condensed with at least one benzene ring; and may be partially saturated. Also, the above heteroaryl herein may be one formed by linking at least one heteroaryl or aryl group to a heteroaryl group via a single bond(s). Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. The term “a fused ring of an (C3-C30) aliphatic ring and an (C6-C30) aromatic ring” in the present disclosure means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of an (C3-C30) aliphatic ring and an (C6-C30) aromatic ring may be replaced with at least one heteroatom selected from B, N, O, S, Si and P, preferably at least one heteroatom selected from N, O and S. The term “halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, i.e., at the 1 and 2 positions on benzene. Meta position is the next substitution position of the immediately adjacent substitution position, i.e., a compound with substituents at the 1 and 3 positions on benzene. Para position is the next substitution position of the meta position, i.e., a compound with substituents at the 1 and 4 positions on benzene.

The term “a ring formed in linking to an adjacent substituent” in the present disclosure means a substituted or unsubstituted (3- to 30-membered) mono- or polycyclic, alicyclic, aromatic ring, or a combination thereof, preferably a substituted or unsubstituted (3- to 26-membered) mono- or polycyclic, alicyclic, aromatic ring, or a combination thereof, formed by linking or fusing two or more adjacent substituents, Further, the formed ring may be included at least one heteroatom selected from the group consisting of B, N, O, S, Si and P, preferably at least one heteroatom selected from the group consisting of N, O and S. According to one embodiment of the present disclosure, the number of atoms in the ring skeleton is 5 to 20; according to another embodiment of the present disclosure, the number of atoms in the ring skeleton is 5 to 15. The fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, the term “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. Preferably, the substituted alkyl, the substituted alkenyl, the substituted cycloalkyl, the substituted aryl(ene), the substituted heteroaryl(ene), the substituted alkoxy, the substituted trialkylsilyl, the substituted dialkylarylsilyl, the substituted alkyldiarylsilyl, the substituted triarylsilyl, and the substituted fused ring of aliphatic ring and aromatic ring in the formulas of the present disclosure, each independently are substituted with least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (C6-C30)aryl unsubstituted or substituted with at least one of (C1-C30)alkyl and di(C6-C30)arylamino; (3- to- 30-membered)heteroaryl unsubstituted or substituted with at least one (C6-C30)aryl; tri(C1-C30)alkylsilyl; tri(C6-C30)arylsilyl; di(C1-C30)alkyl(C6-C30)arylsilyl; (C1-C30)alkyldi(C6-C30)arylsilyl; a fused ring of (C3-C30) aliphatic ring and (C6-C30) aromatic ring; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to- 30-membered)heteroarylamino; (C1-C30)alkyl(C2-C30)alkenylamino; (C1-C30)alkyl(C6-C30)arylamino; (C1-C30)alkyl(3- to-30-membered)heteroarylamino; (C2-C30)alkenyl(C6-C30)arylamino; (C2-C30)alkenyl(3-to- 30-membered)heteroarylamino; (C6-C30)aryl(3- to- 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. For example, the substituents may be substituted with deuterium, cyclohexyl, diphenylamine, naphthyl, anthracenyl, fluorenyl substituted with phenyl, fluoranthenyl, pyridyl substituted with phenyl, imidazolyl substituted with phenyl, or phenoxazinyl, etc.

In the present formula, when there are a plurality of substituents represented by the same symbol, each of the substituents represented by the same symbol may be the same as or different from each other.

Hereinafter, a plurality of host materials according to one embodiment will be described.

A plurality of host materials according to one embodiment comprises at least one first host compound(s) and at least one second host compound(s), wherein the first host compound is represented by Formula 1 and the second host compound is represented by Formula 2.

The first host compound as the host materials according to one embodiment is represented by the following Formula 1.

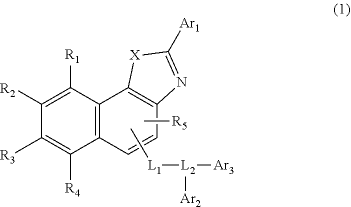

In Formula 1,
X represents —O— or —S—;
R₁ to R₅ each independently represent, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to- 30-membered)heteroaryl, a substituted or unsubstituted (C1-C30)alkoxy, or —NRₐR_{b};
Rₐ and R_{b} each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to- 30-membered)heteroaryl;
L₁ represents a single bond or a substituted or unsubstituted (C6-C30)arylene;
L₂ represents a substituted or unsubstituted nitrogen-containing (3- to- 30-membered)heteroarylene; and
Ar₁ and Ar₃ each independently represent, a substituted or unsubstituted (C6-C30)aryl or a substituted or unsubstituted (3- to- 30-membered)heteroaryl; provided that Ar₂ and Ar₃ are different from each other.

In one embodiment, R₁ to R₅ each independently may be, hydrogen, deuterium, or a substituted or unsubstituted (C6-C30)aryl, preferably hydrogen or a substituted or unsubstituted (C6-C25)aryl, more preferably hydrogen or a substituted or unsubstituted (C6-C18)aryl. For example, R₁ to R₄ each independently may be a substituted or unsubstituted phenyl, a substituted or unsubstituted naphthyl, or a substituted or unsubstituted o-biphenyl, and R₅ may be hydrogen.

In one embodiment, L₁ may be a single bond or a substituted or unsubstituted (C6-C30)arylene, preferably a single bond or a substituted or unsubstituted (C6-C25)arylene, more preferably a single bond or a substituted or unsubstituted (C6-C18)arylene. For example, L₁ may be a single bond, a substituted or unsubstituted phenylene, or a substituted or unsubstituted naphthylene.

In one embodiment, L₂ may be a substituted or unsubstituted (5- to 30-membered)heteroarylene containing at least one nitrogen (N), preferably a substituted or unsubstituted (5- to 25-membered)heteroarylene containing at least two nitrogens, more preferably a substituted or unsubstituted (5- to 18-membered)heteroarylene containing at least three nitrogens. For example, L₂ may be a substituted or unsubstituted pyridylene, a substituted or unsubstituted pyrimidylene, a substituted or unsubstituted triazinylene, a substituted or unsubstituted quinazolinylene, a substituted or unsubstituted quinoxalinylene, a substituted or unsubstituted benzoquinazolinylene, or a substituted or unsubstituted benzoquinoxalinylene. For example, L₂ may be a substituted or unsubstituted pyridylene, a substituted or unsubstituted pyrimidylene, a substituted or unsubstituted triazinylene, a substituted or unsubstituted quinazolinylene, or a substituted or unsubstituted quinoxalinylene.

In one embodiment, Ar₁ may be a substituted or unsubstituted (C6-C30)aryl. For example, Arₙ may be phenyl unsubstituted or substituted with at least one deuterium, a substituted or unsubstituted naphthyl, a substituted or unsubstituted p-biphenyl, a substituted or unsubstituted o-biphenyl, or a substituted or unsubstituted phenanthrenyl.

In one embodiment, Ar₂ and Ar₃ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl, wherein Ar₂ and Ar₃ are different from each other. For example, Ar₂ and Ar₃ each independently may be phenyl unsubstituted or substituted with at least one deuterium or adamantanyl, a substituted or unsubstituted naphthyl, a substituted or unsubstituted o-biphenyl, a substituted or unsubstituted m-biphenyl, a substituted or unsubstituted phenanthrenyl, dibenzofuranyl unsubstituted or substituted with at least one of deuterium; and phenyl, a substituted or unsubstituted benzonaphthofuranyl, a substituted or unsubstituted dinaphthofuranyl, a substituted or unsubstituted dibenzothiophenyl, or a substituted or unsubstituted dibenzoselenophenyl.

In one embodiment, at least one of Ar₂ and Ar₃ may be a substituted or unsubstituted (5- to 30-membered)heteroaryl. For example, at least one of Ar₂ and Ar₃ may be a substituted or unsubstituted dibenzofuranyl, a substituted or unsubstituted dibenzothiophenyl, or a substituted or unsubstituted dibenzoselenophenyl.

According to one embodiment, the compound represented by Formula 1 above may be more specifically illustrated by the following compounds, but is not limited thereto.

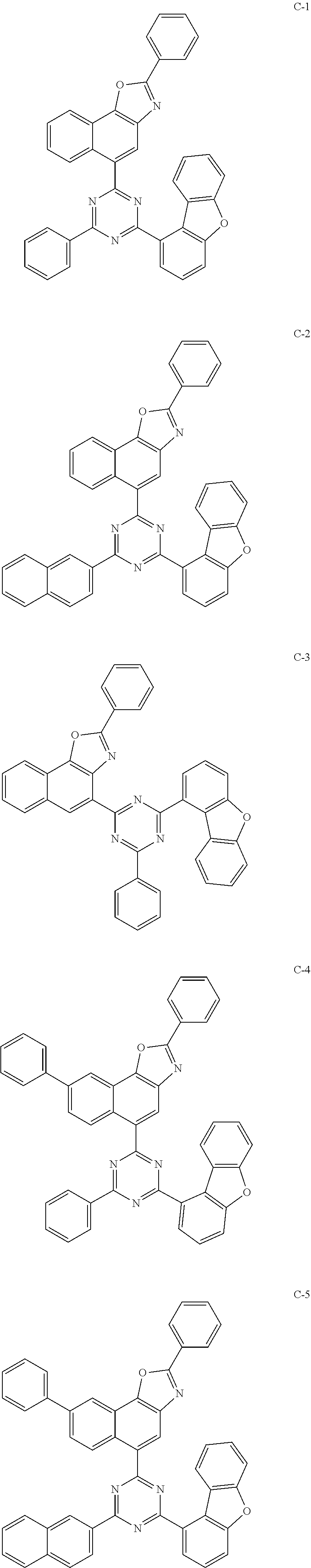 [SMILES: c1ccc(-c2nc(-c3cc4nc(-c5ccccc5)oc4c4ccccc34)nc(-c3cccc4oc5ccccc5c34)n2)cc1 ; c1ccc(-c2nc3cc(-c4nc(-c5ccc6ccccc6c5)nc(-c5cccc6oc7ccccc7c56)n4)c4ccccc4c3o2)cc1 ; c1ccc(-c2nc(-c3cc4ccccc4c4oc(-c5ccccc5)nc34)nc(-c3cccc4oc5ccccc5c34)n2)cc1 ; c1ccc(-c2ccc3c(-c4nc(-c5ccccc5)nc(-c5cccc6oc7ccccc7c56)n4)cc4nc(-c5ccccc5)oc4c3c2)cc1 ; c1ccc(-c2ccc3c(-c4nc(-c5ccc6ccccc6c5)nc(-c5cccc6oc7ccccc7c56)n4)cc4nc(-c5ccccc5)oc4c3c2)cc1]

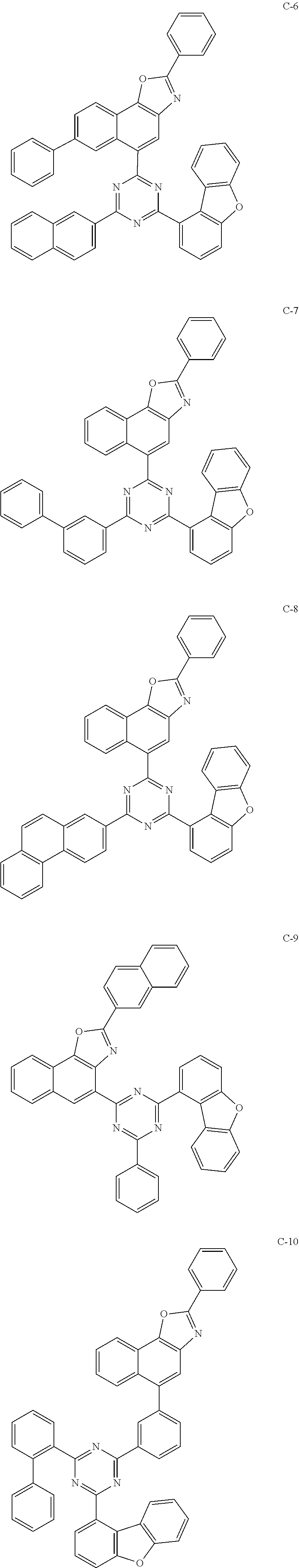 [SMILES: c1ccc(-c2ccc3c(c2)c(-c2nc(-c4ccc5ccccc5c4)nc(-c4cccc5oc6ccccc6c45)n2)cc2nc(-c4ccccc4)oc23)cc1 ; c1ccc(-c2cccc(-c3nc(-c4cc5nc(-c6ccccc6)oc5c5ccccc45)nc(-c4cccc5oc6ccccc6c45)n3)c2)cc1 ; c1ccc(-c2nc3cc(-c4nc(-c5ccc6c(ccc7ccccc76)c5)nc(-c5cccc6oc7ccccc7c56)n4)c4ccccc4c3o2)cc1 ; c1ccc(-c2nc(-c3cc4ccccc4c4oc(-c5ccc6ccccc6c5)nc34)nc(-c3cccc4oc5ccccc5c34)n2)cc1 ; c1ccc(-c2nc3cc(-c4cccc(-c5nc(-c6ccccc6-c6ccccc6)nc(-c6cccc7oc8ccccc8c67)n5)c4)c4ccccc4c3o2)cc1]

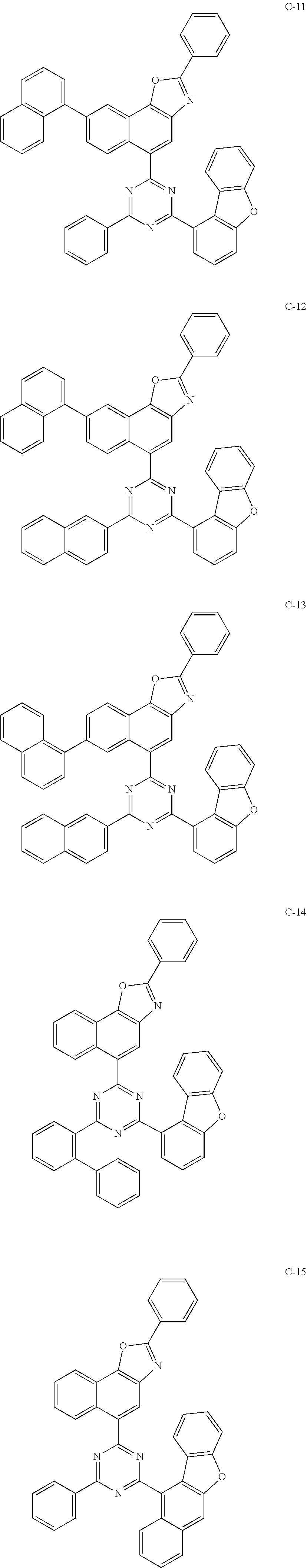 [SMILES: c1ccc(-c2nc(-c3cc4nc(-c5ccccc5)oc4c4cc(-c5cccc6ccccc56)ccc34)nc(-c3cccc4oc5ccccc5c34)n2)cc1 ; c1ccc(-c2nc3cc(-c4nc(-c5ccc6ccccc6c5)nc(-c5cccc6oc7ccccc7c56)n4)c4ccc(-c5cccc6ccccc56)cc4c3o2)cc1 ; c1ccc(-c2nc3cc(-c4nc(-c5ccc6ccccc6c5)nc(-c5cccc6oc7ccccc7c56)n4)c4cc(-c5cccc6ccccc56)ccc4c3o2)cc1 ; c1ccc(-c2nc3cc(-c4nc(-c5ccccc5-c5ccccc5)nc(-c5cccc6oc7ccccc7c56)n4)c4ccccc4c3o2)cc1 ; c1ccc(-c2nc(-c3cc4nc(-c5ccccc5)oc4c4ccccc34)nc(-c3c4ccccc4cc4oc5ccccc5c34)n2)cc1]

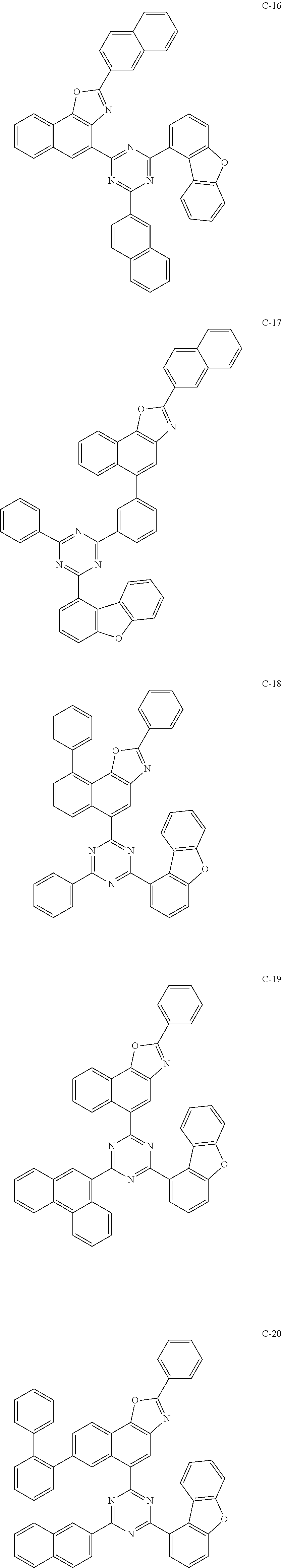 [SMILES: c1ccc2cc(-c3nc(-c4cc5ccccc5c5oc(-c6ccc7ccccc7c6)nc45)nc(-c4cccc5oc6ccccc6c45)n3)ccc2c1 ; c1ccc(-c2nc(-c3cccc(-c4cc5nc(-c6ccc7ccccc7c6)oc5c5ccccc45)c3)nc(-c3cccc4oc5ccccc5c34)n2)cc1 ; c1ccc(-c2nc(-c3cc4nc(-c5ccccc5)oc4c4c(-c5ccccc5)cccc34)nc(-c3cccc4oc5ccccc5c34)n2)cc1 ; c1ccc(-c2nc3cc(-c4nc(-c5cc6ccccc6c6ccccc56)nc(-c5cccc6oc7ccccc7c56)n4)c4ccccc4c3o2)cc1 ; c1ccc(-c2nc3cc(-c4nc(-c5ccc6ccccc6c5)nc(-c5cccc6oc7ccccc7c56)n4)c4cc(-c5ccccc5-c5ccccc5)ccc4c3o2)cc1]

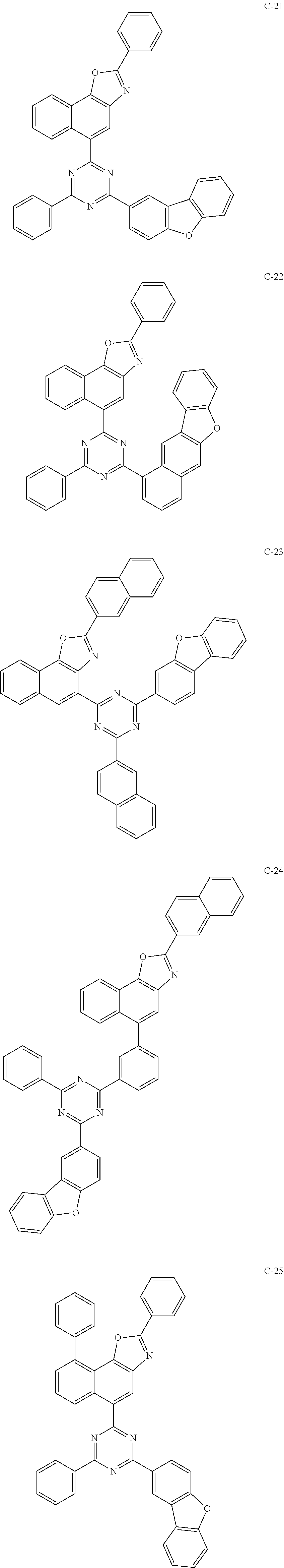 [SMILES: c1ccc(-c2nc(-c3ccc4oc5ccccc5c4c3)nc(-c3cc4nc(-c5ccccc5)oc4c4ccccc34)n2)cc1 ; c1ccc(-c2nc(-c3cccc4cc5oc6ccccc6c5cc34)nc(-c3cc4nc(-c5ccccc5)oc4c4ccccc34)n2)cc1 ; c1ccc2cc(-c3nc(-c4ccc5c(c4)oc4ccccc45)nc(-c4cc5ccccc5c5oc(-c6ccc7ccccc7c6)nc45)n3)ccc2c1 ; c1ccc(-c2nc(-c3cccc(-c4cc5nc(-c6ccc7ccccc7c6)oc5c5ccccc45)c3)nc(-c3ccc4oc5ccccc5c4c3)n2)cc1 ; c1ccc(-c2nc(-c3ccc4oc5ccccc5c4c3)nc(-c3cc4nc(-c5ccccc5)oc4c4c(-c5ccccc5)cccc34)n2)cc1]

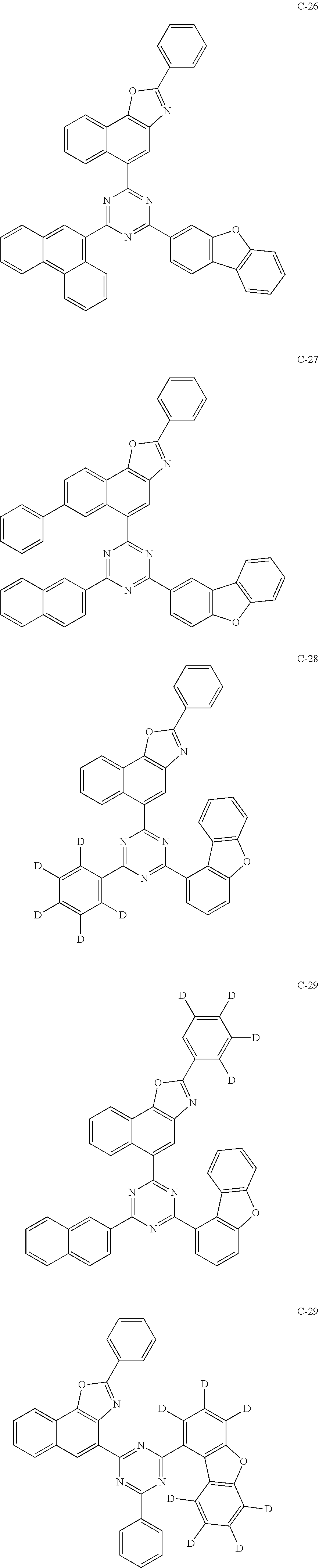

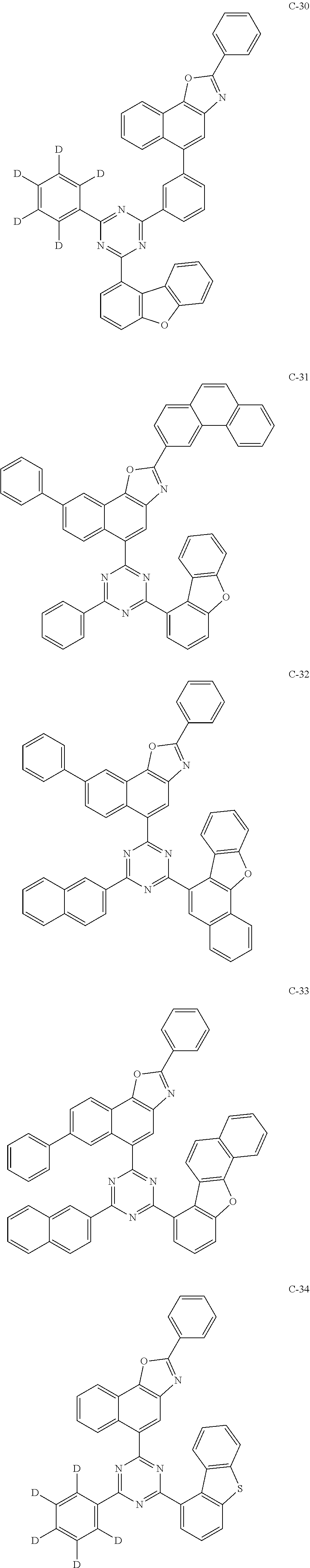

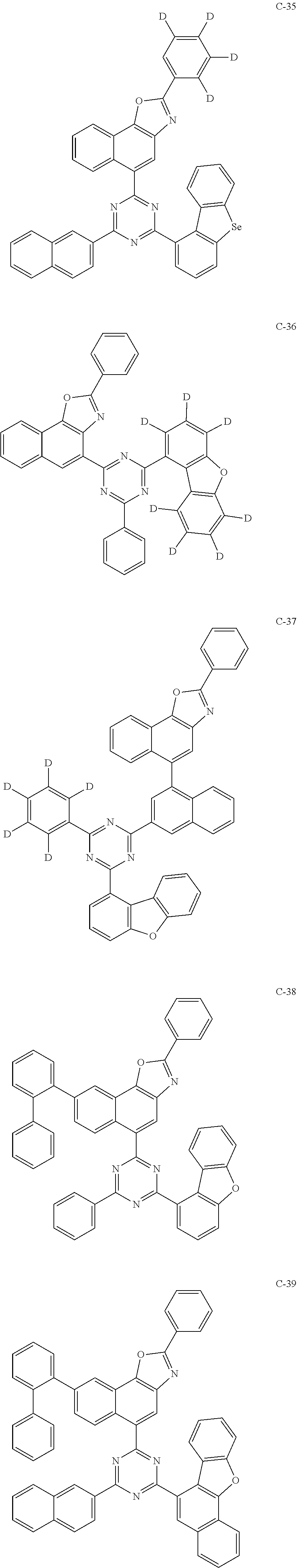

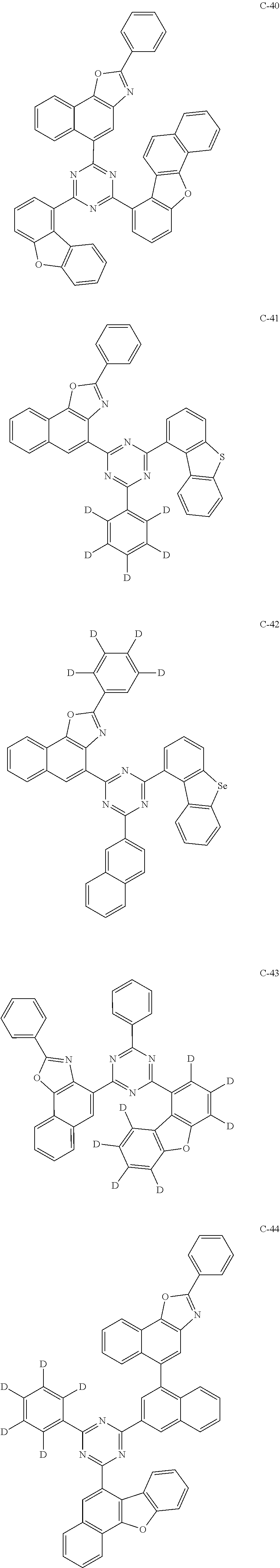

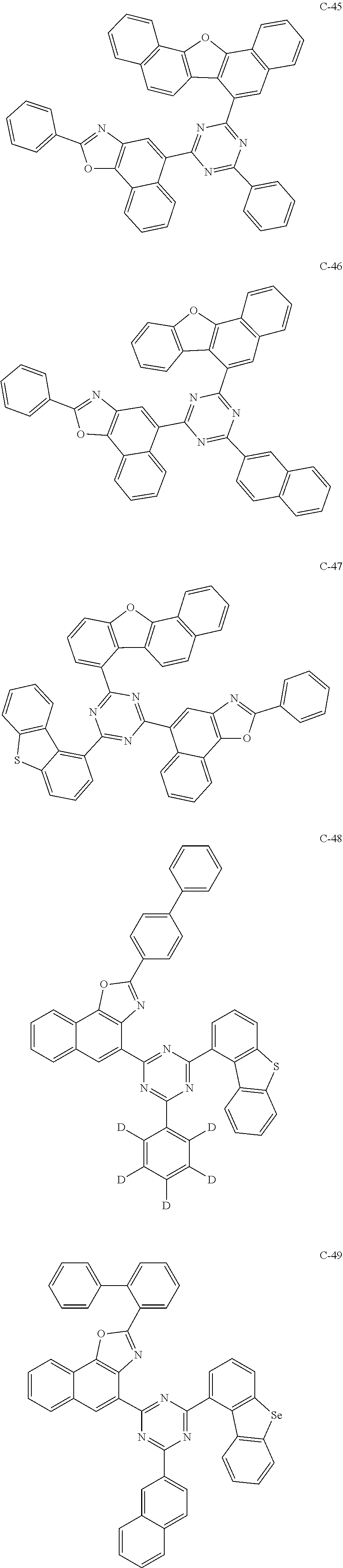 [SMILES: c1ccc(-c2nc(-c3cc4nc(-c5ccccc5)oc4c4ccccc34)nc(-c3cc4ccccc4c4oc5c6ccccc6ccc5c34)n2)cc1 ; c1ccc(-c2nc3cc(-c4nc(-c5ccc6ccccc6c5)nc(-c5cc6ccccc6c6oc7ccccc7c56)n4)c4ccccc4c3o2)cc1 ; c1ccc(-c2nc3cc(-c4nc(-c5cccc6oc7c8ccccc8ccc7c56)nc(-c5cccc6sc7ccccc7c56)n4)c4ccccc4c3o2)cc1 ; [2H]c1c([2H])c([2H])c(-c2nc(-c3cc4ccccc4c4oc(-c5ccc(-c6ccccc6)cc5)nc34)nc(-c3cccc4sc5ccccc5c34)n2)c([2H])c1[2H] ; c1ccc(-c2ccccc2-c2nc3c(-c4nc(-c5ccc6ccccc6c5)nc(-c5cccc6[se]c7ccccc7c56)n4)cc4ccccc4c3o2)cc1]

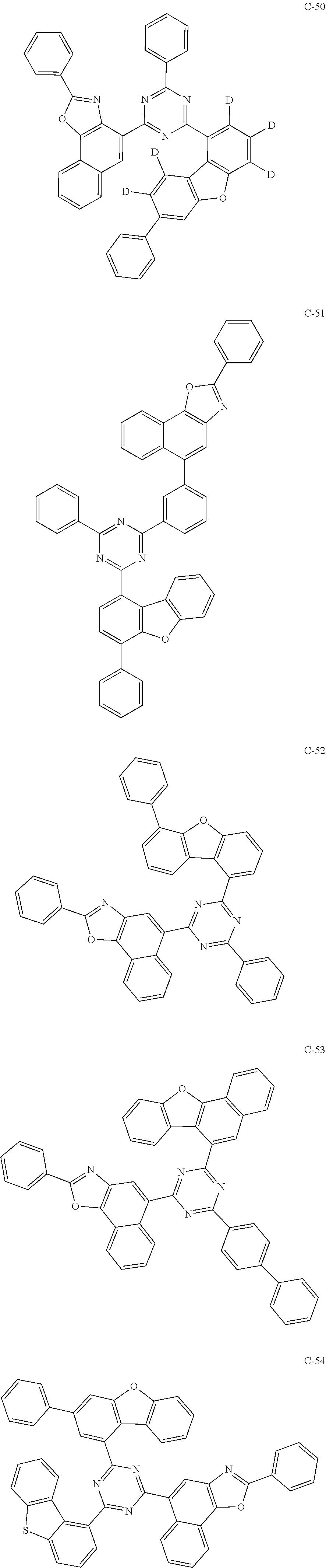 [SMILES: [2H]c1c([2H])c(-c2nc(-c3ccccc3)nc(-c3cc4ccccc4c4oc(-c5ccccc5)nc34)n2)c2c(oc3cc(-c4ccccc4)c([2H])c([2H])c32)c1[2H] ; c1ccc(-c2nc(-c3cccc(-c4cc5nc(-c6ccccc6)oc5c5ccccc45)c3)nc(-c3ccc(-c4ccccc4)c4oc5ccccc5c34)n2)cc1 ; c1ccc(-c2nc(-c3cc4nc(-c5ccccc5)oc4c4ccccc34)nc(-c3cccc4oc5c(-c6ccccc6)cccc5c34)n2)cc1 ; c1ccc(-c2ccc(-c3nc(-c4cc5nc(-c6ccccc6)oc5c5ccccc45)nc(-c4cc5ccccc5c5oc6ccccc6c45)n3)cc2)cc1 ; c1ccc(-c2cc(-c3nc(-c4cc5nc(-c6ccccc6)oc5c5ccccc45)nc(-c4cccc5sc6ccccc6c45)n3)c3c(c2)oc2ccccc23)cc1]

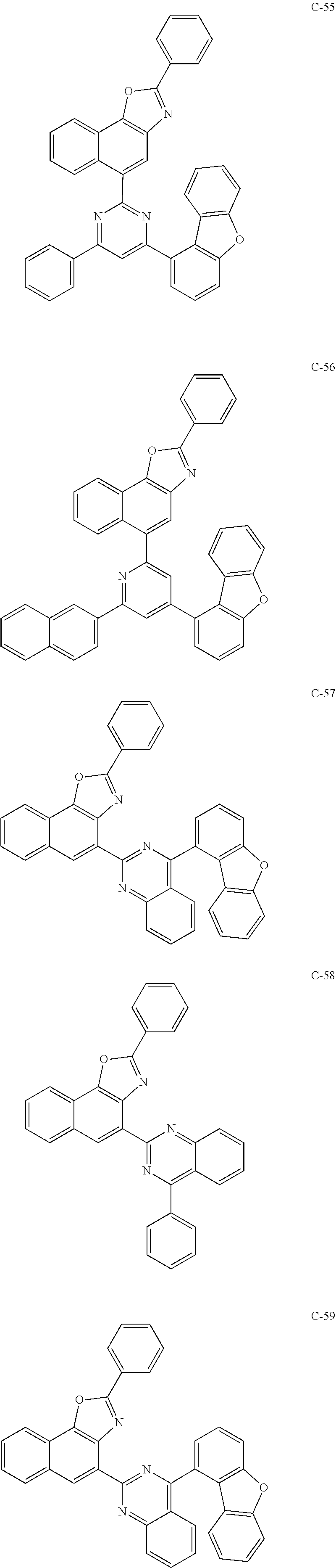 [SMILES: c1ccc(-c2cc(-c3cccc4oc5ccccc5c34)nc(-c3cc4nc(-c5ccccc5)oc4c4ccccc34)n2)cc1 ; c1ccc(-c2nc3cc(-c4cc(-c5cccc6oc7ccccc7c56)cc(-c5ccc6ccccc6c5)n4)c4ccccc4c3o2)cc1 ; c1ccc(-c2nc3c(-c4nc(-c5cccc6oc7ccccc7c56)c5ccccc5n4)cc4ccccc4c3o2)cc1 ; c1ccc(-c2nc3c(-c4nc(-c5ccccc5)c5ccccc5n4)cc4ccccc4c3o2)cc1 ; c1ccc(-c2nc3c(-c4nc(-c5cccc6oc7ccccc7c56)c5ccccc5n4)cc4ccccc4c3o2)cc1]

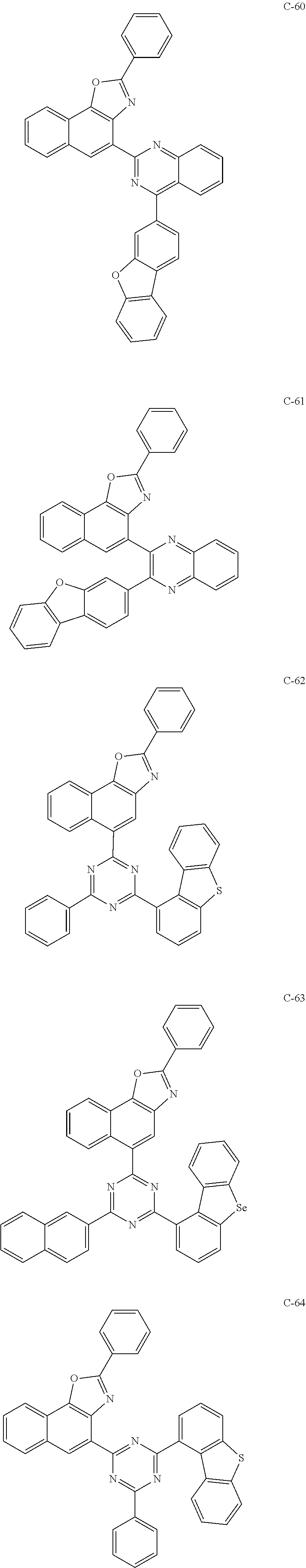 [SMILES: c1ccc(-c2nc3c(-c4nc(-c5ccc6c(c5)oc5ccccc56)c5ccccc5n4)cc4ccccc4c3o2)cc1 ; c1ccc(-c2nc3c(-c4nc5ccccc5nc4-c4ccc5c(c4)oc4ccccc45)cc4ccccc4c3o2)cc1 ; c1ccc(-c2nc(-c3cc4nc(-c5ccccc5)oc4c4ccccc34)nc(-c3cccc4sc5ccccc5c34)n2)cc1 ; c1ccc(-c2nc3cc(-c4nc(-c5ccc6ccccc6c5)nc(-c5cccc6[se]c7ccccc7c56)n4)c4ccccc4c3o2)cc1 ; c1ccc(-c2nc(-c3cc4ccccc4c4oc(-c5ccccc5)nc34)nc(-c3cccc4sc5ccccc5c34)n2)cc1]

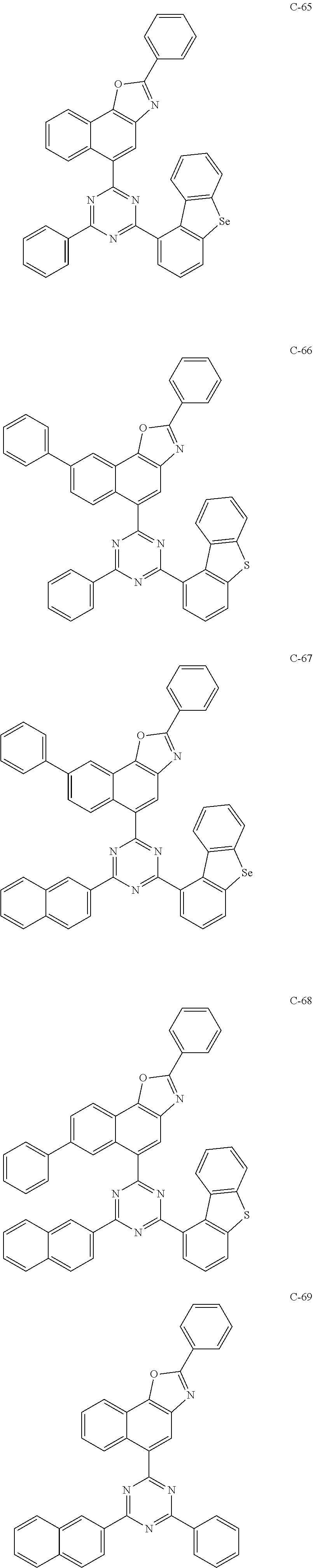

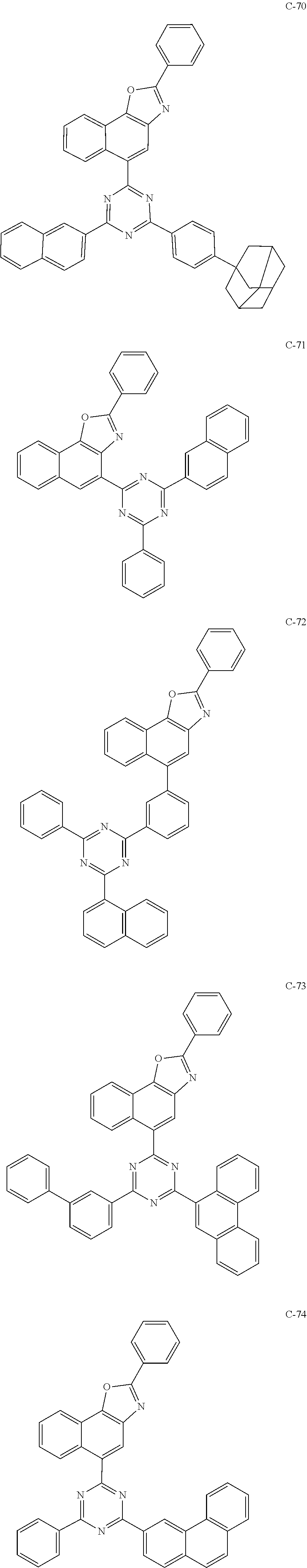 [SMILES: c1ccc(-c2nc3cc(-c4nc(-c5ccc(C67CC8CC9CC(C6)C98C7)cc5)nc(-c5ccc6ccccc6c5)n4)c4ccccc4c3o2)cc1 ; c1ccc(-c2nc(-c3ccc4ccccc4c3)nc(-c3cc4ccccc4c4oc(-c5ccccc5)nc34)n2)cc1 ; c1ccc(-c2nc(-c3cccc(-c4cc5nc(-c6ccccc6)oc5c5ccccc45)c3)nc(-c3cccc4ccccc34)n2)cc1 ; c1ccc(-c2cccc(-c3nc(-c4cc5ccccc5c5ccccc45)nc(-c4cc5nc(-c6ccccc6)oc5c5ccccc45)n3)c2)cc1 ; c1ccc(-c2nc(-c3ccc4ccc5ccccc5c4c3)nc(-c3cc4nc(-c5ccccc5)oc4c4ccccc34)n2)cc1]

The compound represented by Formula 1 according to the present disclosure may be prepared as shown in the following Reaction Scheme 1, but is not limited thereto, and may also be prepared by synthetic methods known to those skilled in the art.

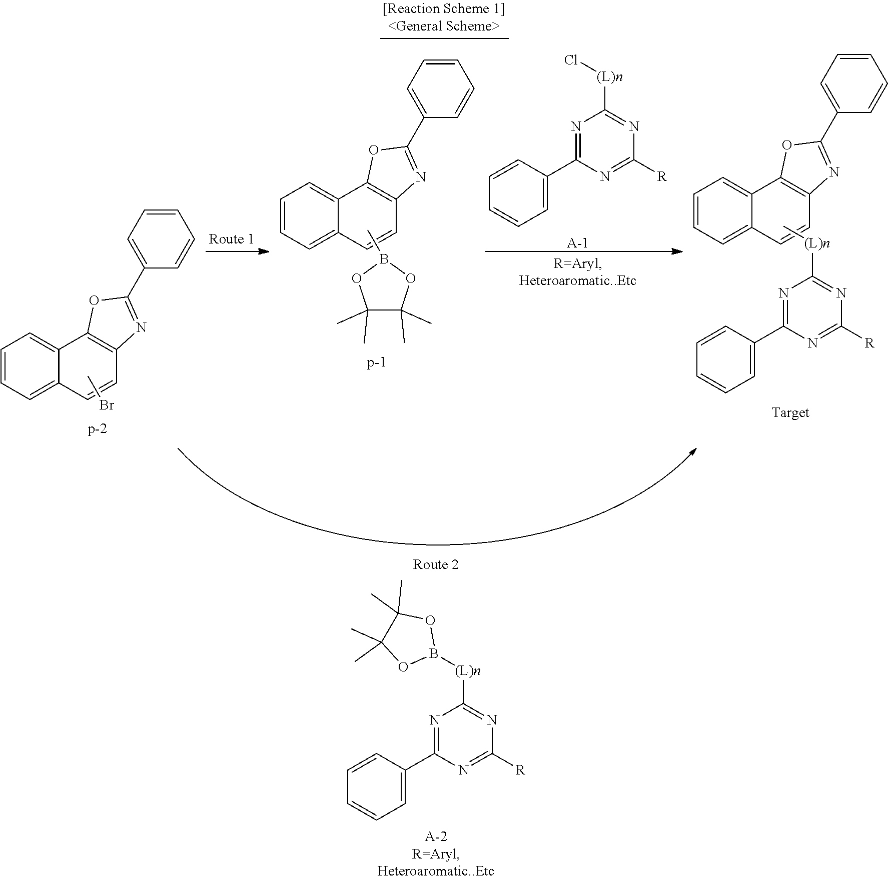

As described above, exemplary synthesis examples of the compounds represented by formula 1 are explained, but they are based on Buchwald-Hartwig cross coupling reaction, N-arylation reaction, H-mont-mediated etherification reaction, Miyaura borylation reaction, Suzuki cross-coupling reaction, intramolecular acid-induced cyclization reaction, Pd(II)-catalyzed oxidative cyclization reaction, Grignard reaction, Heck reaction, cyclic dehydration reaction, SN₁ substitution reaction, SN₂ substitution reaction, and phosphine-mediated reductive cyclization reaction etc. It will be understood by one skilled in the art that the above reaction proceeds even if other substituents defined in Formula 1 other than the substituents described in the specific synthesis examples, are bonded.

A second host compound, which is another host material according to one embodiment, is a compound represented by the following Formula 2.

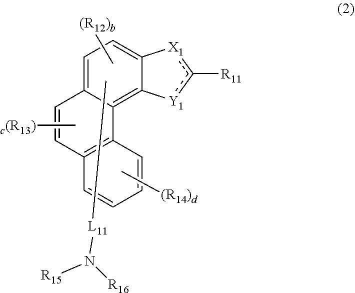

In Formula 2,
X₁ and Y₁ each independently represent, —N═, —NR_{c}, —O— or —S—, provided that any one of X₁ and Y₁ is —N═, and the other of X₁ and Y₁ is —NR_{c}—, —O— or —S—;
R₁₁ represents a substituted or unsubstituted (C6-C30)aryl or a substituted or unsubstituted (3- to- 30-membered)heteroaryl;
R₁₂ to R₁₄, and R_{c} each independently represent, hydrogen, deuterium, halogen, cyano, a substituted or unsubstituted (C1-C30)alkyl, a substituted or unsubstituted (C6-C30)aryl, a substituted or unsubstituted (3- to- 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -L₁₂-N(Ar₁₁)(Ar₁₂); or may be linked to the adjacent substituents to form a ring(s);
R₁₅ and R₁₆ each independently represent, a substituted or unsubstituted (C6-C30)aryl or a substituted or unsubstituted (3- to- 30-membered)heteroaryl;
L₁₁ and L₁₂ each independently represent, a single bond, a substituted or unsubstituted (C6-C30)arylene, or a substituted or unsubstituted (3- to- 30-membered)heteroarylene;
Ar₁₁ and Ar₁₂ each independently represent, hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (3- to- 30-membered)heteroaryl; and
b and c each independently represent, an integer of 1 or 2, and d represents an integer of 1 to 4, and when b to d are an integer of 2 or more, each of R₁₂ to R₁₄ may be the same or different.

In one embodiment, X₁ and Y₁ each independently may be —N═, —NR_{c}, —O—, or —S—, For example, X₁ may be —N═ and Y₁ may be —O— or —S—; or X₁ may be —O— or —S—, and Y₁ may be —N═.

In one embodiment, R₁₁ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R₁₁ may be a substituted or unsubstituted phenyl, a substituted or unsubstituted naphthyl, a substituted or unsubstituted o-biphenyl, a substituted or unsubstituted p-biphenyl, a substituted or unsubstituted phenanthrenyl, or a substituted or unsubstituted pyridyl.

In one embodiment, R₁₂ to R₁₄ each independently may be hydrogen, deuterium, halogen, cyano, or a substituted or unsubstituted (C6-C30)aryl; or may be linked to the adjacent substituents to form a ring(s), preferably hydrogen, deuterium, or a substituted or unsubstituted (C6-C25)aryl; or may be linked to the adjacent substituents to form a substituted or unsubstituted (5- to 30-membered) monocyclic or polycyclic aromatic ring(s), more preferably hydrogen or a substituted or unsubstituted (C6-C18)aryl; or may be linked to the adjacent substituents to form a substituted or unsubstituted (5- to 30-membered) monocyclic aromatic ring(s). For example, R₁₂ to R₁₄ each independently may be hydrogen or a substituted or unsubstituted phenyl; or may be linked to the adjacent substituents to form a benzene ring(s).

In one embodiment, L₁₁ may be a single bond, a substituted or unsubstituted (C6-C30)arylene, or a substituted or unsubstituted (5- to 30-membered)heteroarylene, preferably a single bond or a substituted or unsubstituted (C6-C25)arylene, more preferably a single bond or a substituted or unsubstituted (C6-C18)arylene. For example, L₁₁ may be a single bond or a substituted or unsubstituted phenylene.

In one embodiment, R₁₅ and R₁₆ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl. For example, R₁₅ and R₁₆ each independently may be phenyl unsubstituted or substituted with at least one selected from cyclohexyl; diphenylamine; naphthyl; anthracenyl; fluorenyl substituted with phenyl; fluoranthenyl; pyridyl substituted with phenyl; imidazolyl substituted with phenyl; and phenoxazinyl, naphthyl unsubstituted or substituted with phenyl, a substituted or unsubstituted phenanthrenyl, p-biphenyl unsubstituted or substituted with at least one selected from deuterium; methy and tert-butyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted o-terphenyl, a substituted or unsubstituted o-tetraphenyl, a substituted or unsubstituted dimethylfluorenyl, a substituted or unsubstituted diphenylfluorenyl, a substituted or unsubstituted benzofluorenyl, a substituted or unsubstituted spirobifluorenyl, a substituted or unsubstituted 9,9,10,10-tetramethylphenanthrenyl, C22aryl, a substituted or unsubstituted benzothiophenyl, carbazolyl unsubstituted or substituted with phenyl, dibenzofuranyl unsubstituted or substituted with phenyl, a substituted or unsubstituted naphthobenzofuranyl, a substituted or unsubstituted dibenzothiophenyl, or a substituted or unsubstituted naphthobenzothiophenyl.

According to one embodiment, the compound represented by Formula 2 above may be more specifically illustrated by the following compounds, but is not limited thereto.

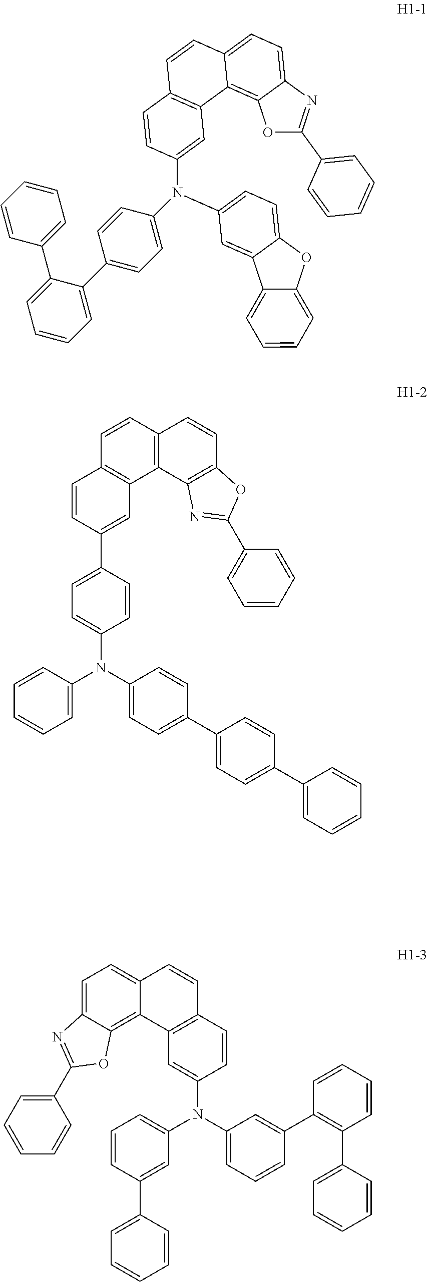 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

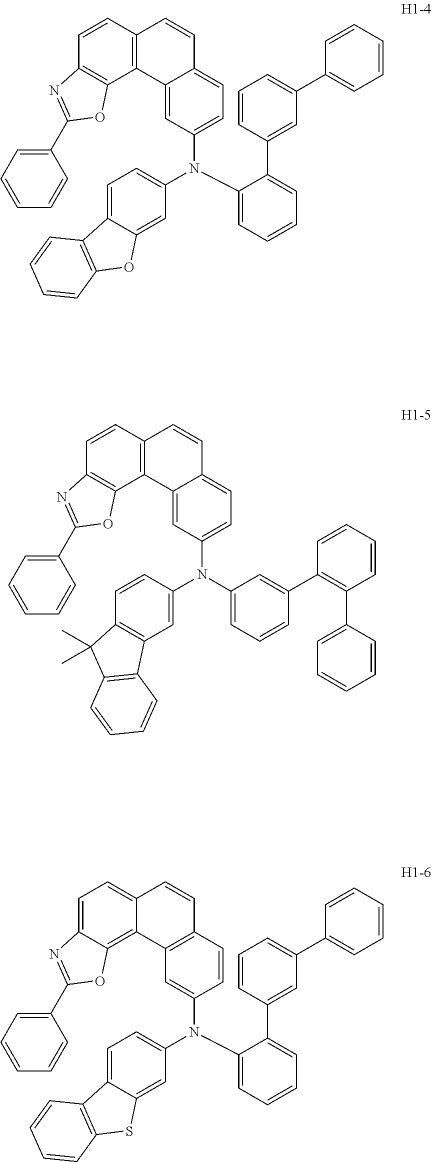 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

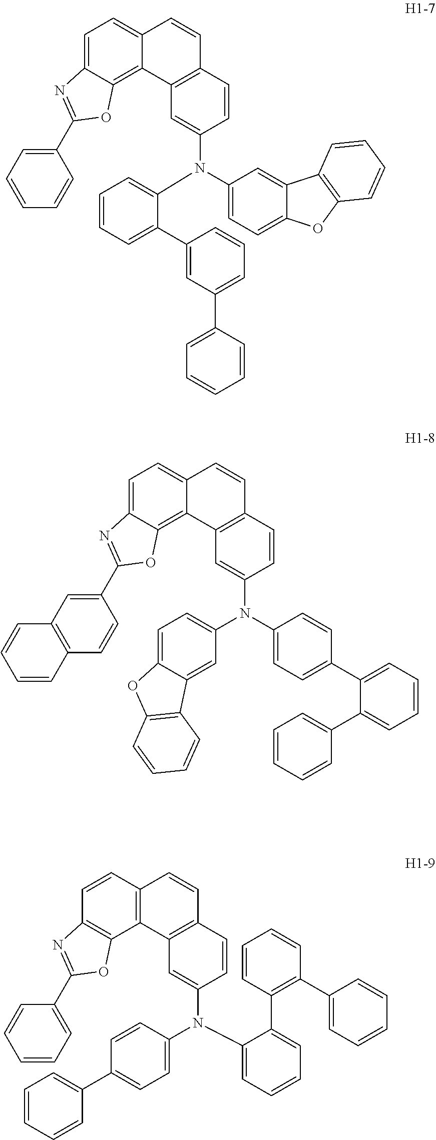 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1]

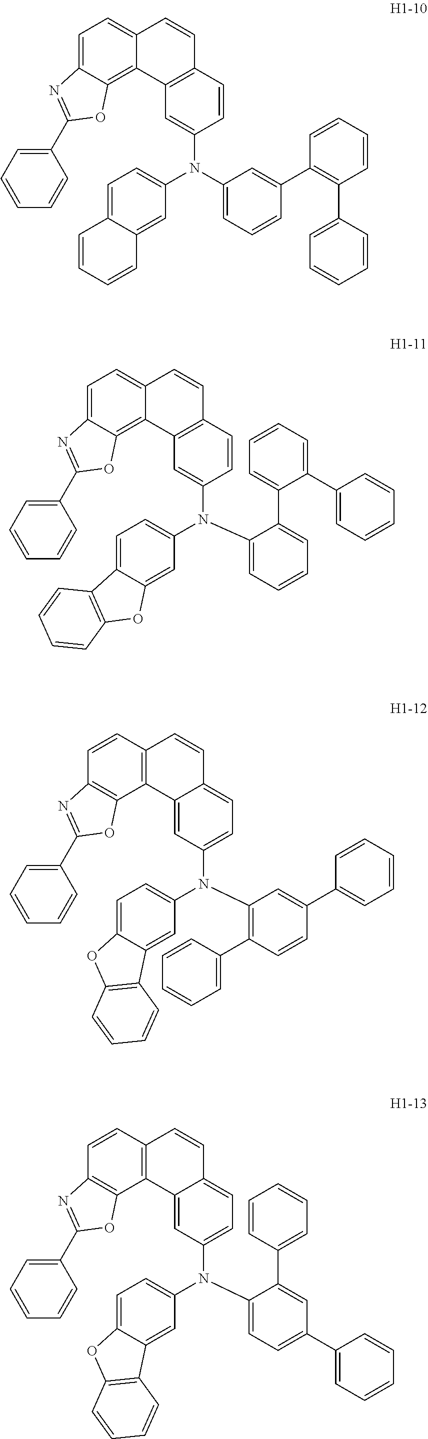 [SMILES: c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1]

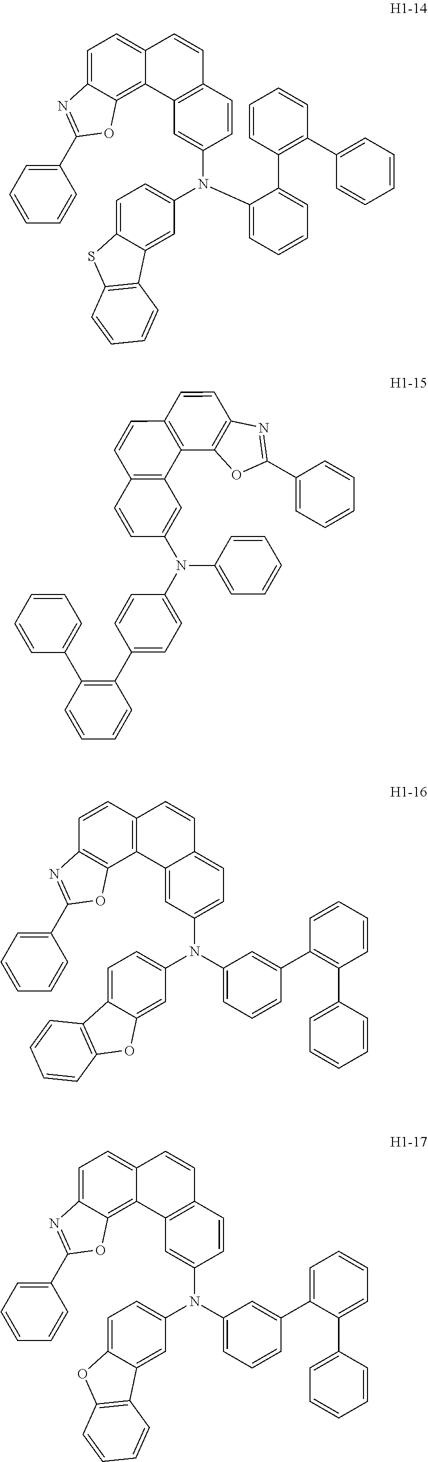 [SMILES: c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

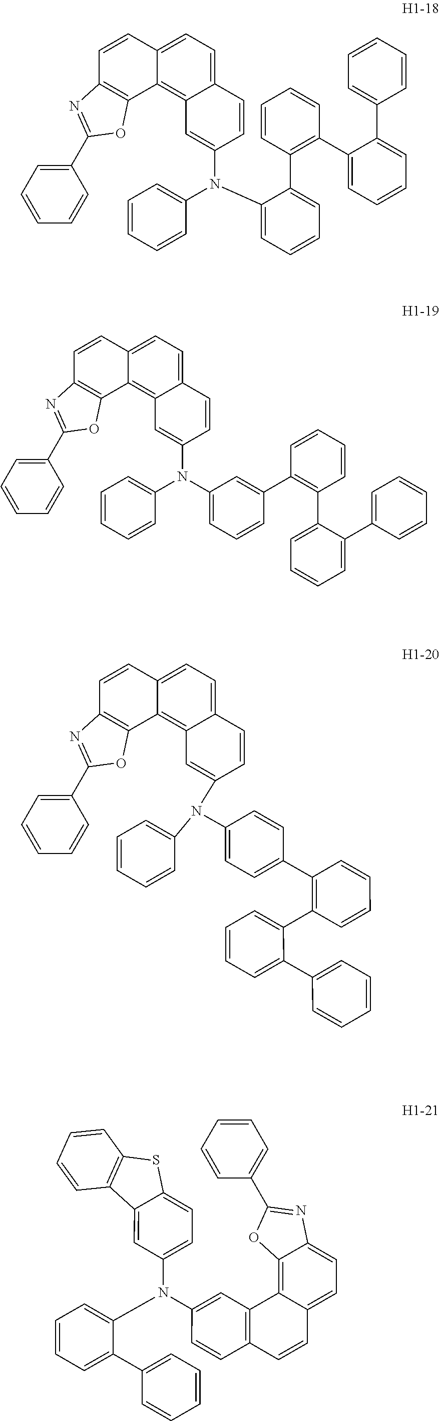 [SMILES: c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccc7sc8ccccc8c7c6)c6ccccc6-c6ccccc6)cc5c4c3o2)cc1]

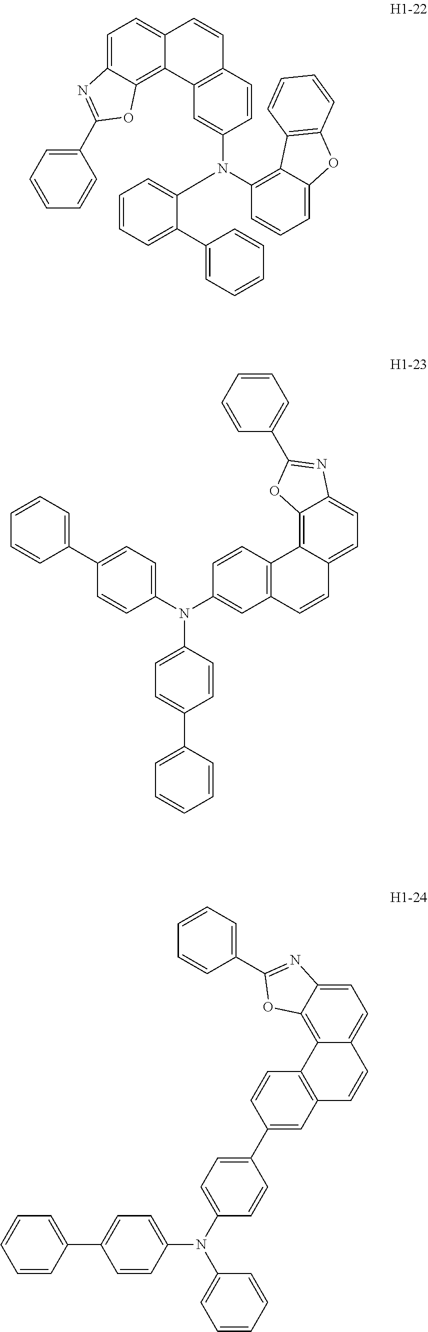 [SMILES: c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1]

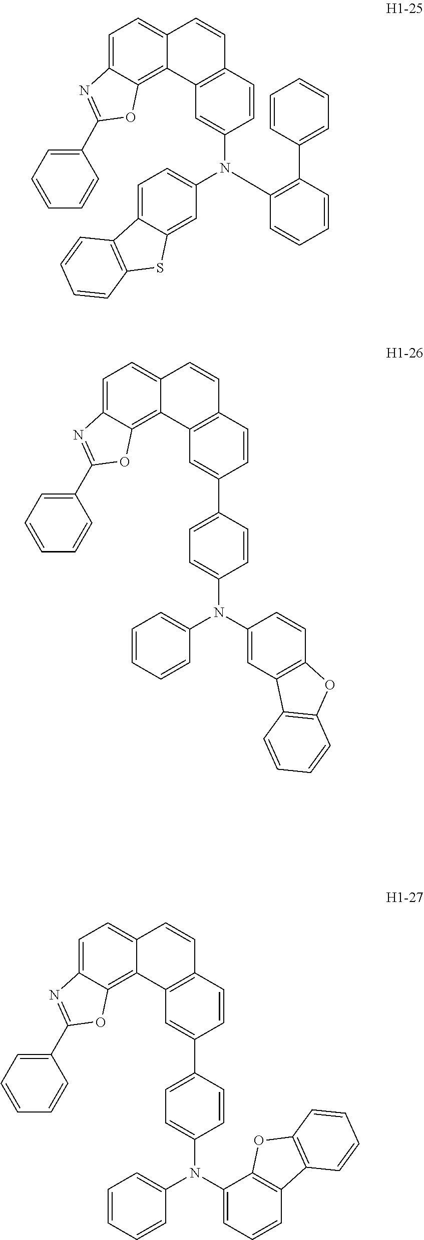 [SMILES: c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1 ; c1ccc(-c2nc3ccc4ccc5ccc(-c6ccc(N(c7ccccc7)c7cccc8c7oc7ccccc78)cc6)cc5c4c3o2)cc1]

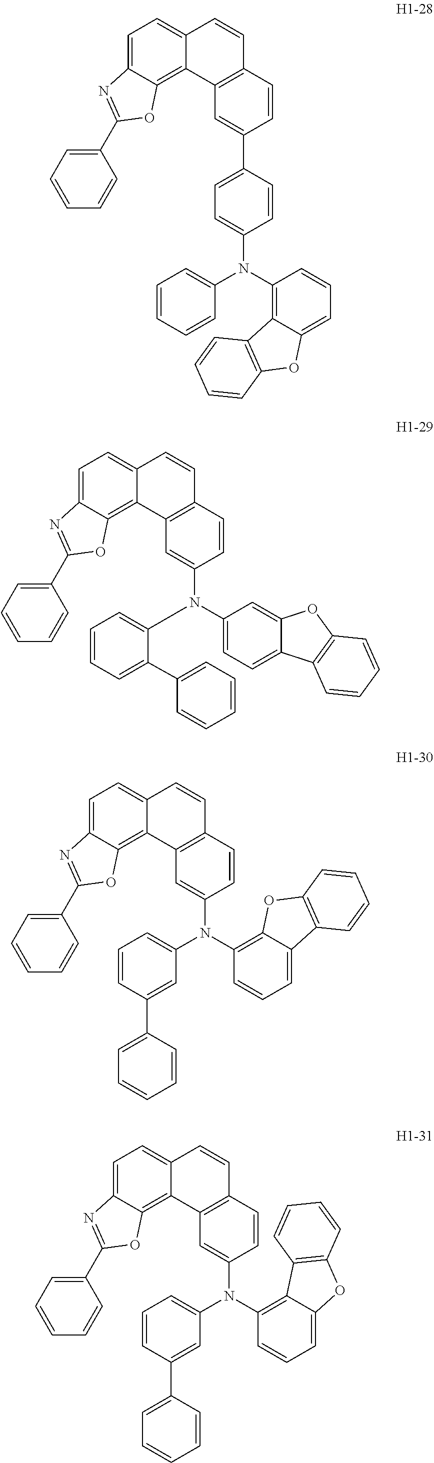

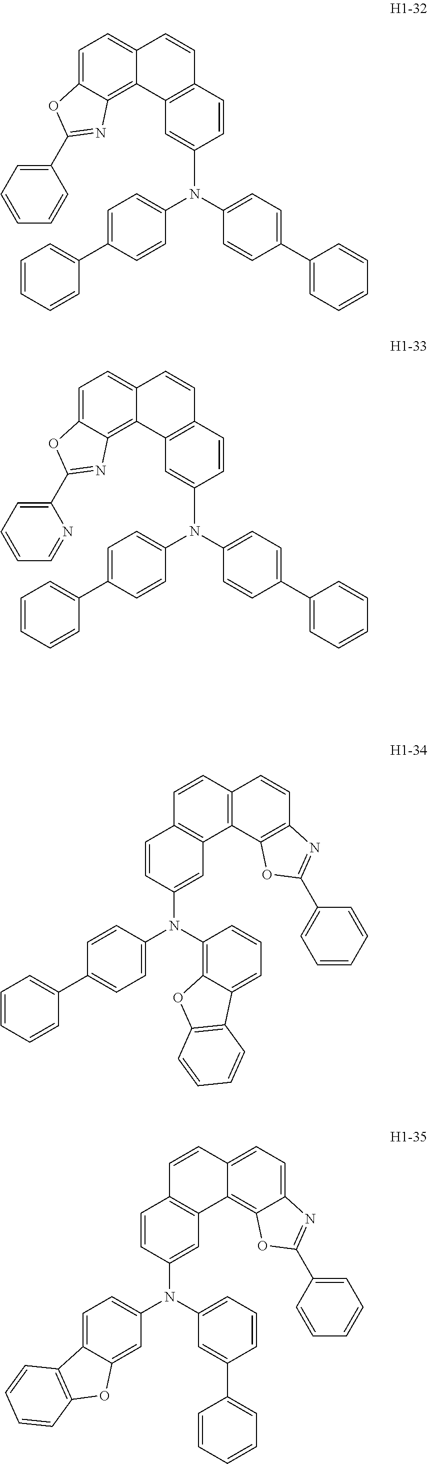 [SMILES: c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1]

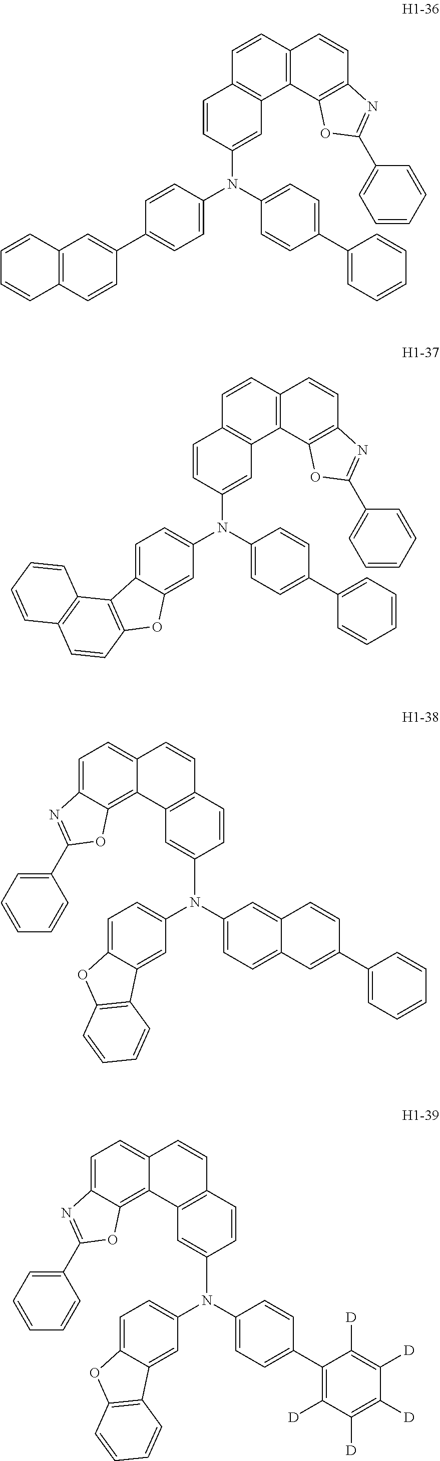 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H]]

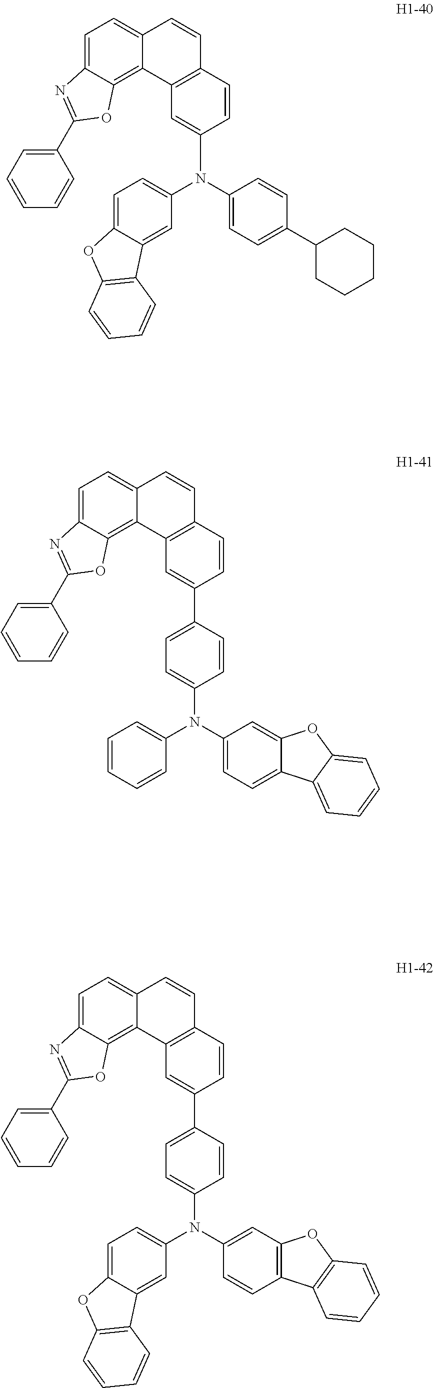

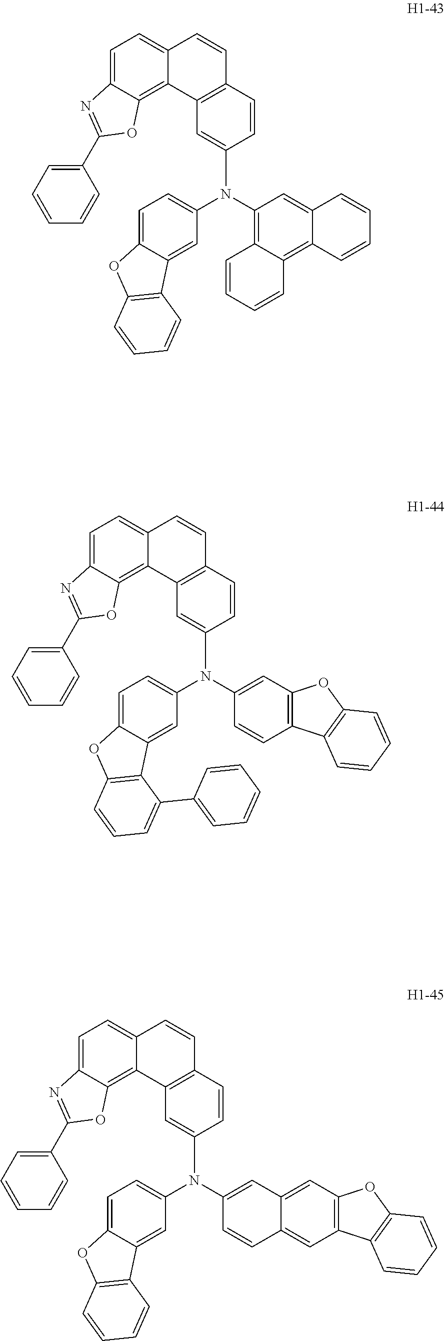 [SMILES: c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

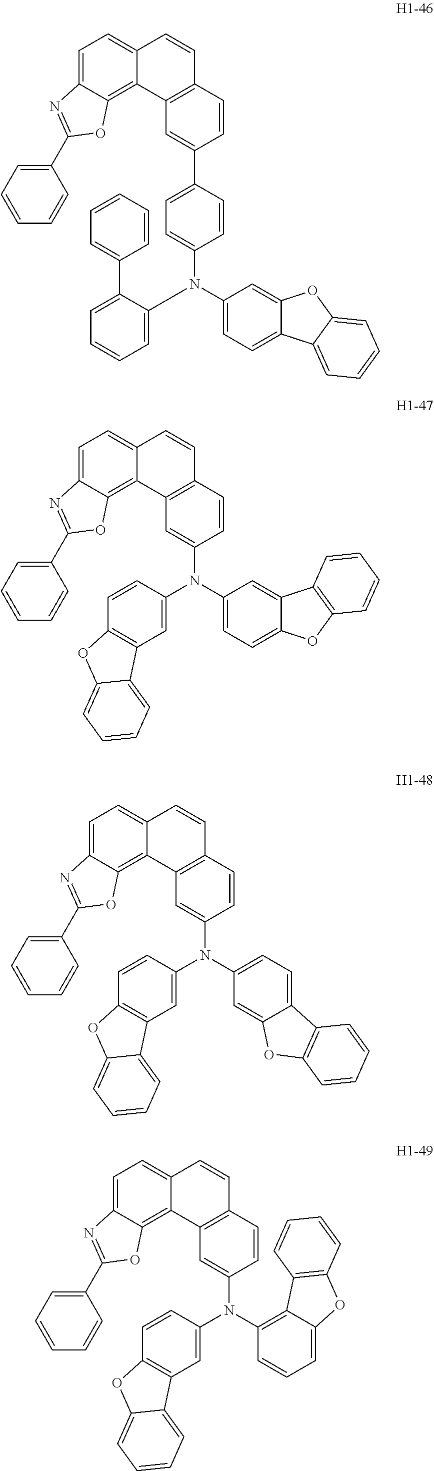 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

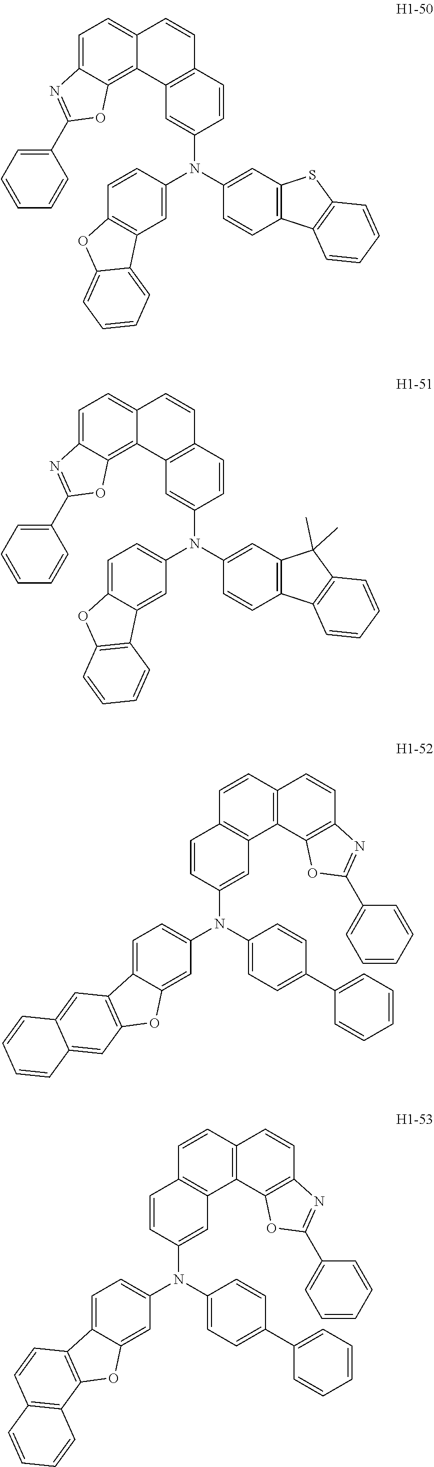 [SMILES: c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1]

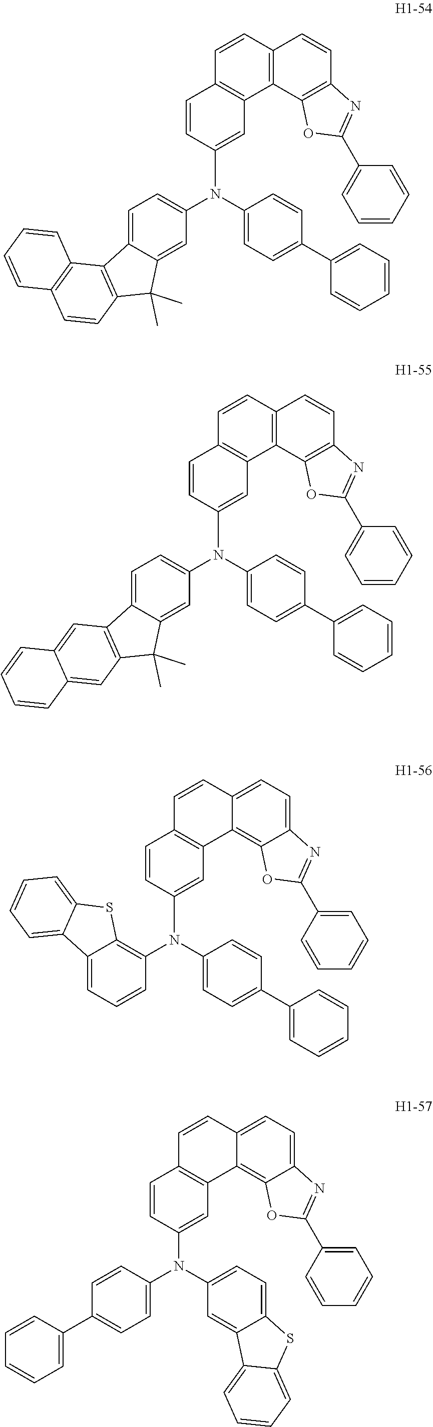 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

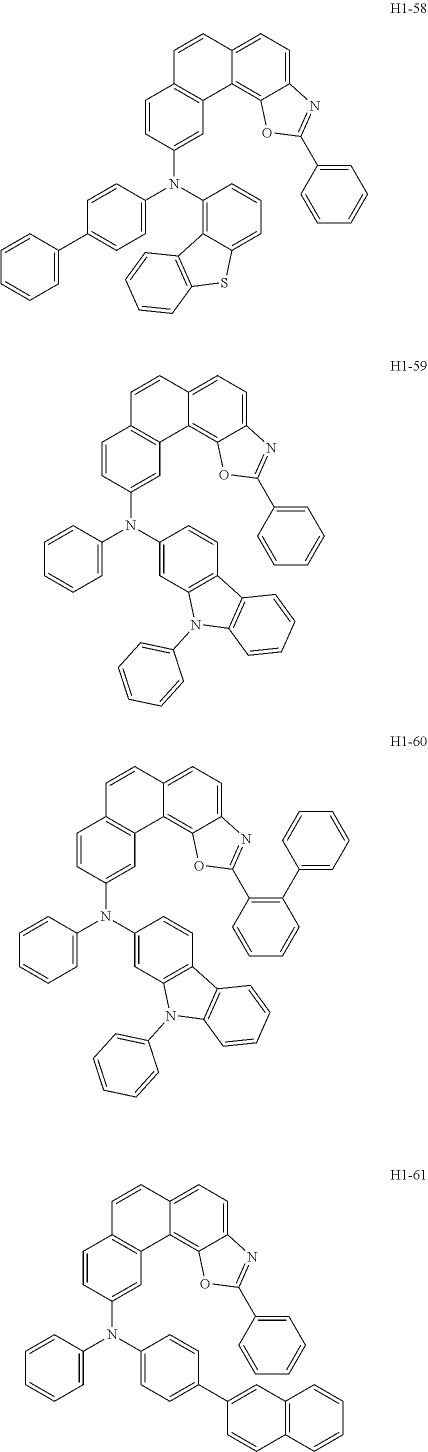 [SMILES: c1ccc(-c2ccc(N(c3ccc4ccc5ccc6nc(-c7ccccc7)oc6c5c4c3)c3cccc4sc5ccccc5c34)cc2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc(-c7ccc8ccccc8c7)cc6)cc5c4c3o2)cc1]

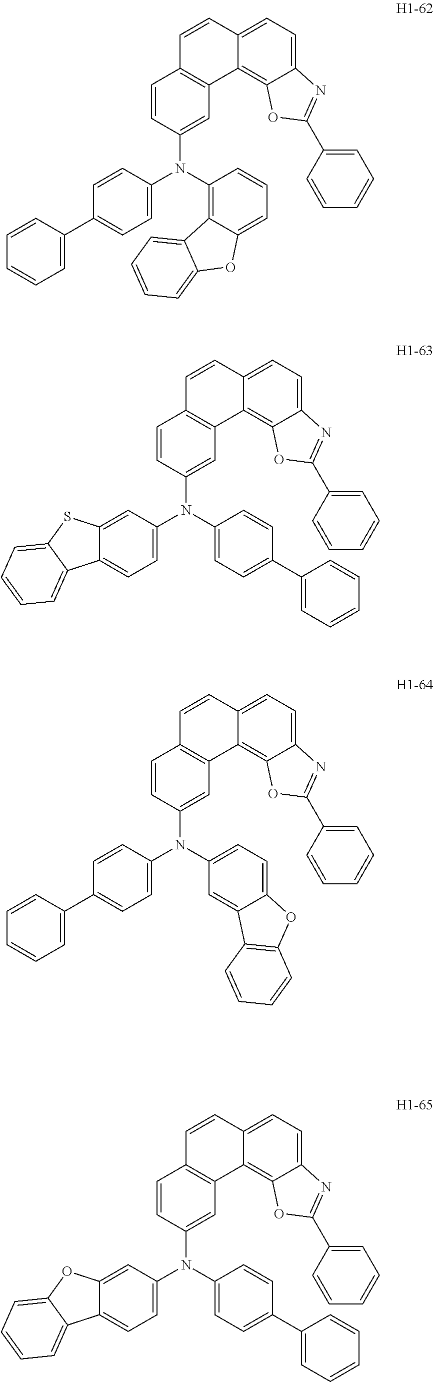 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1]

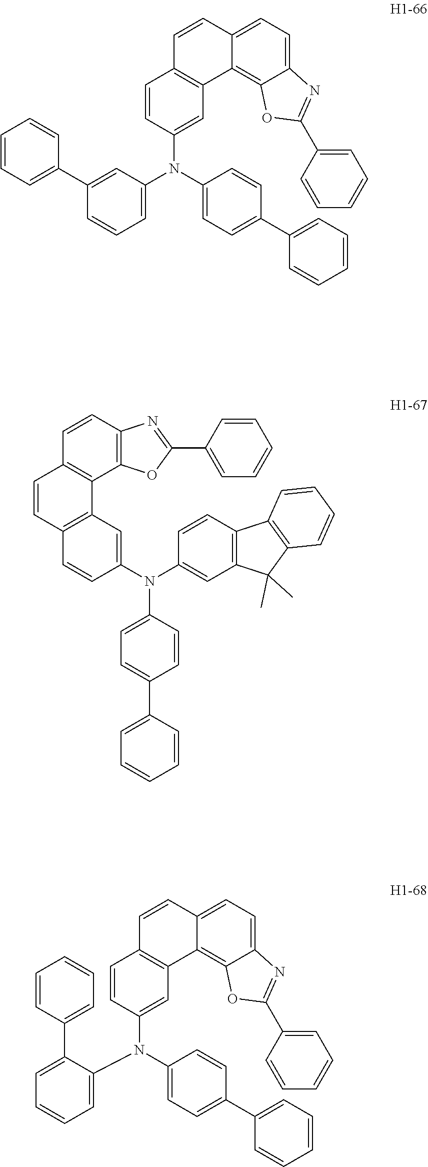 [SMILES: c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

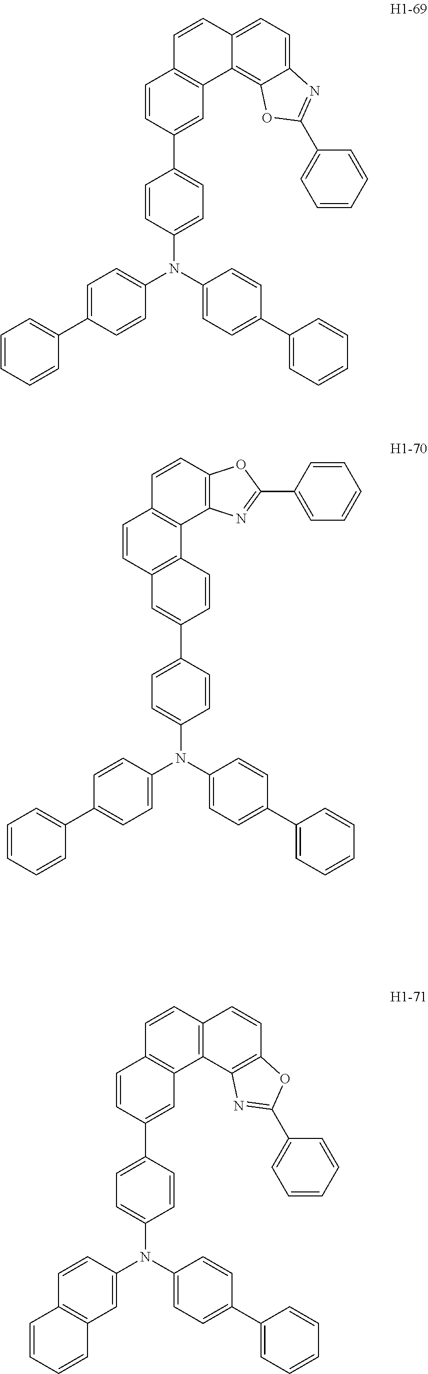 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

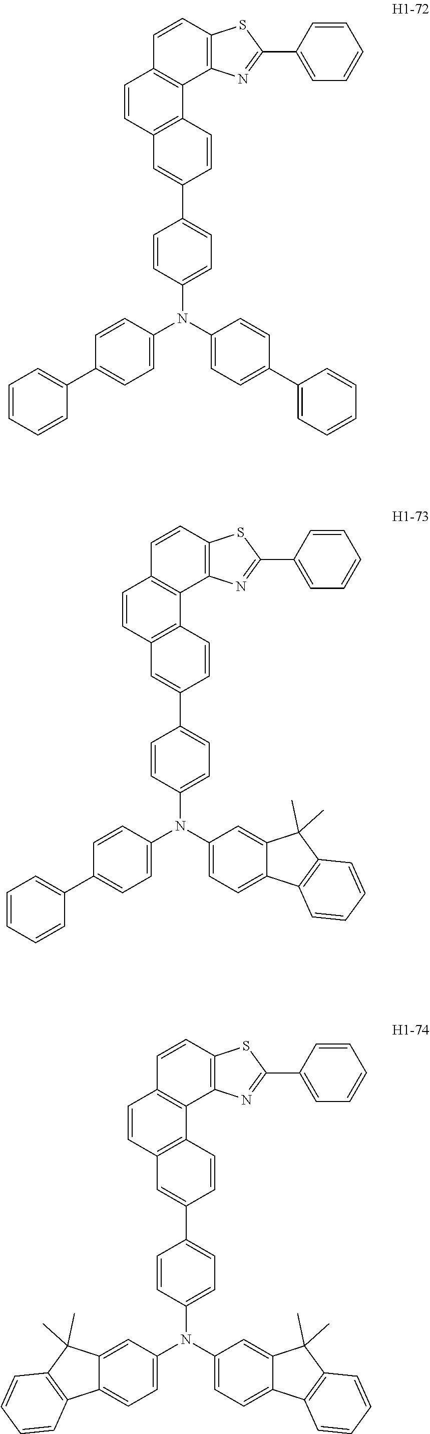 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

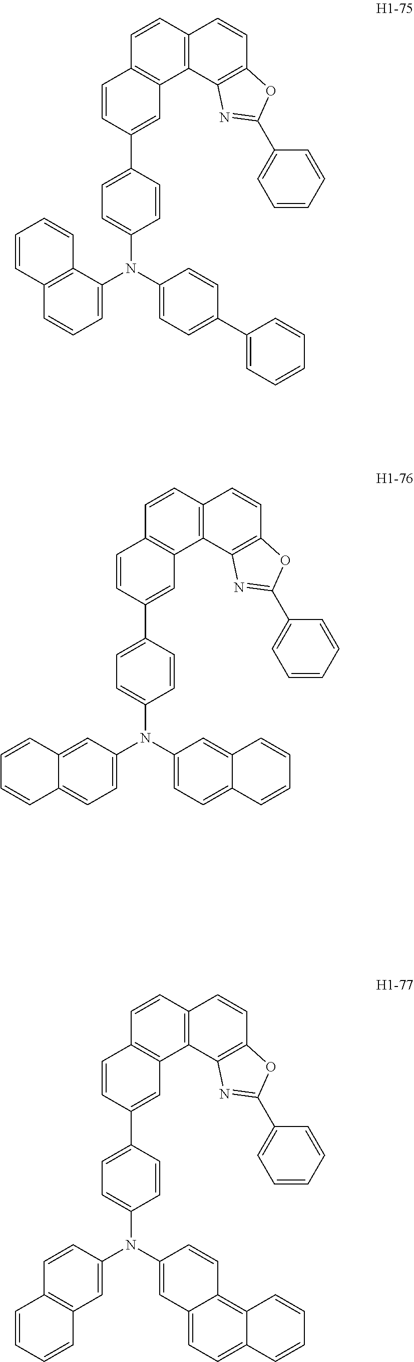 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

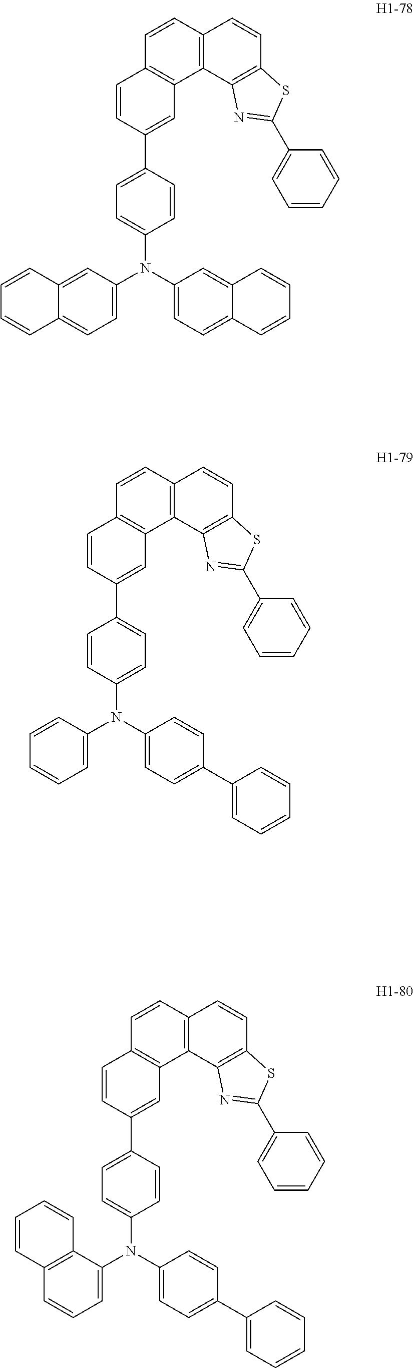 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

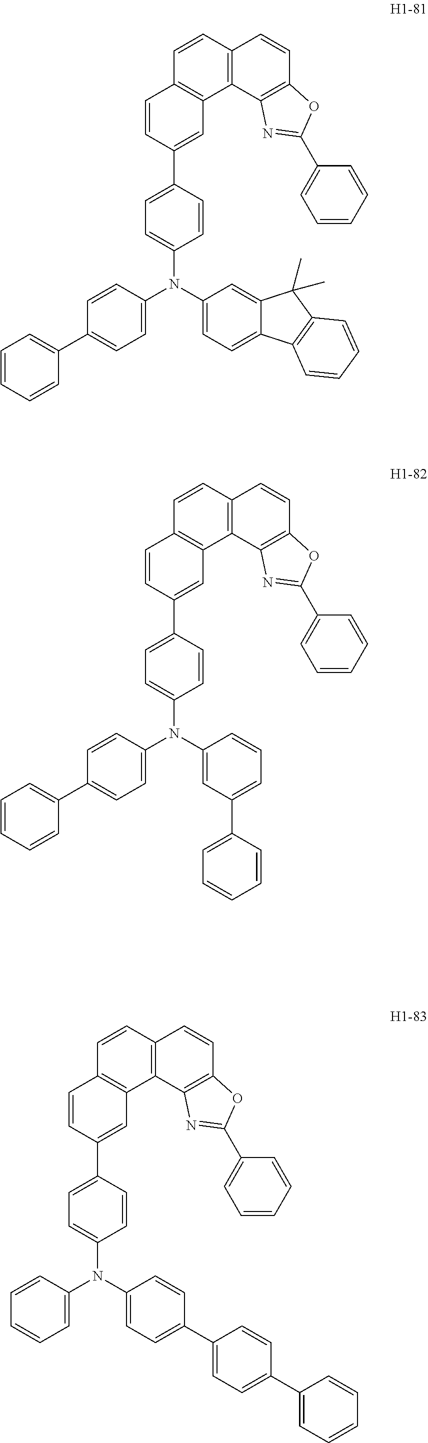 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

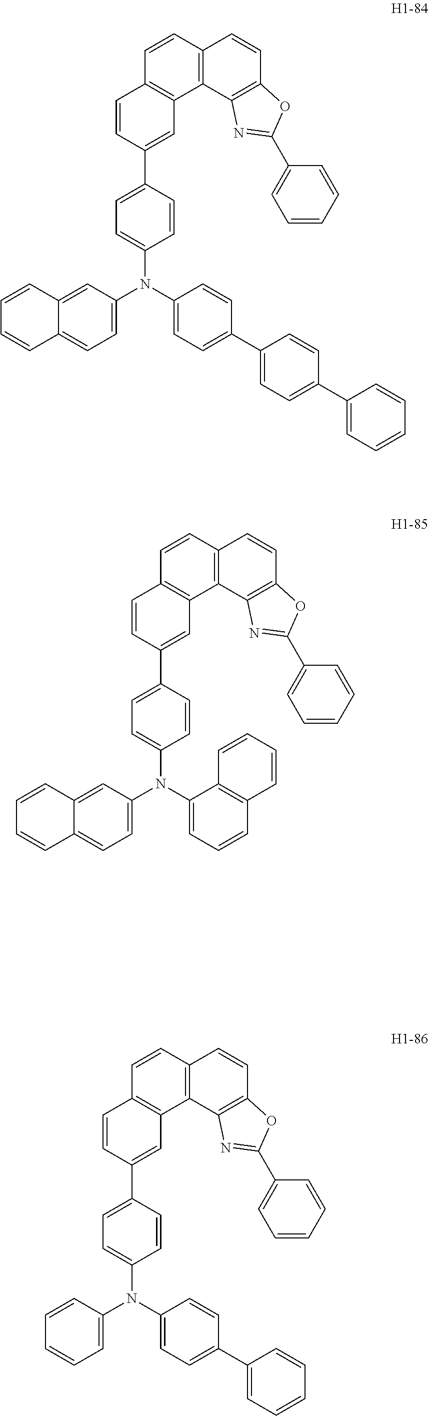 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

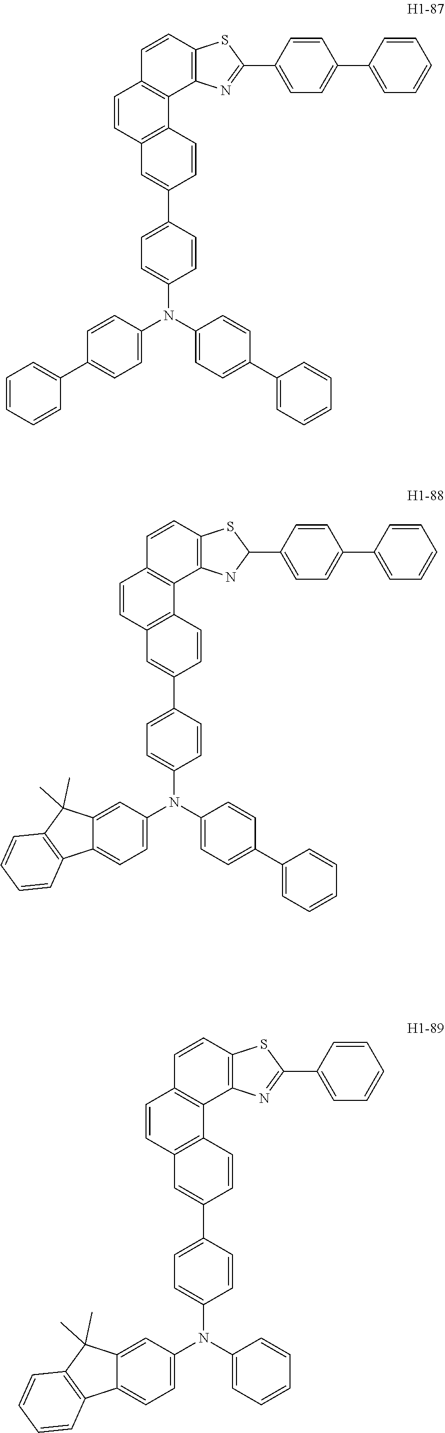

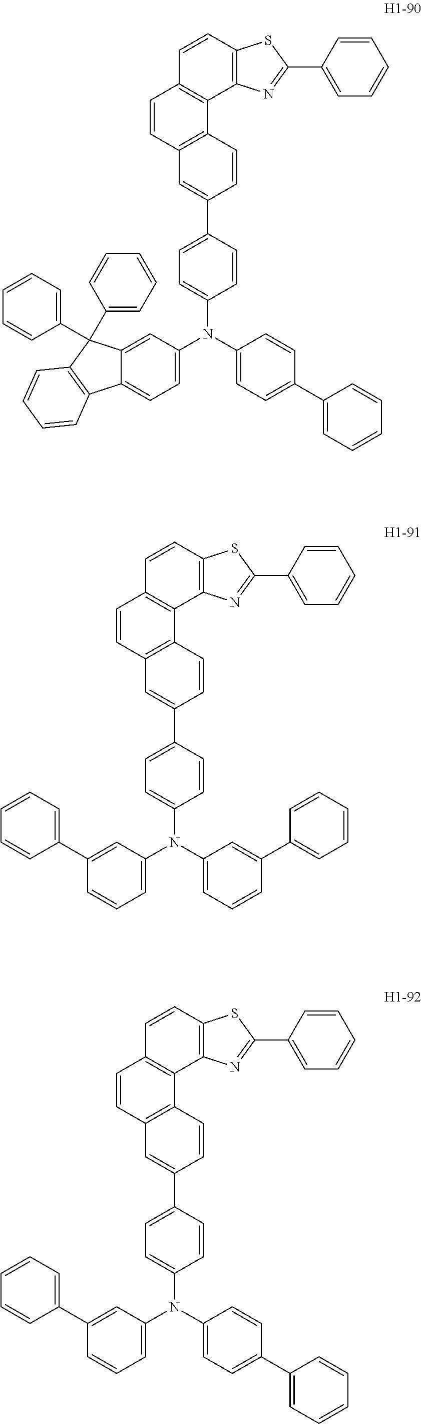 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

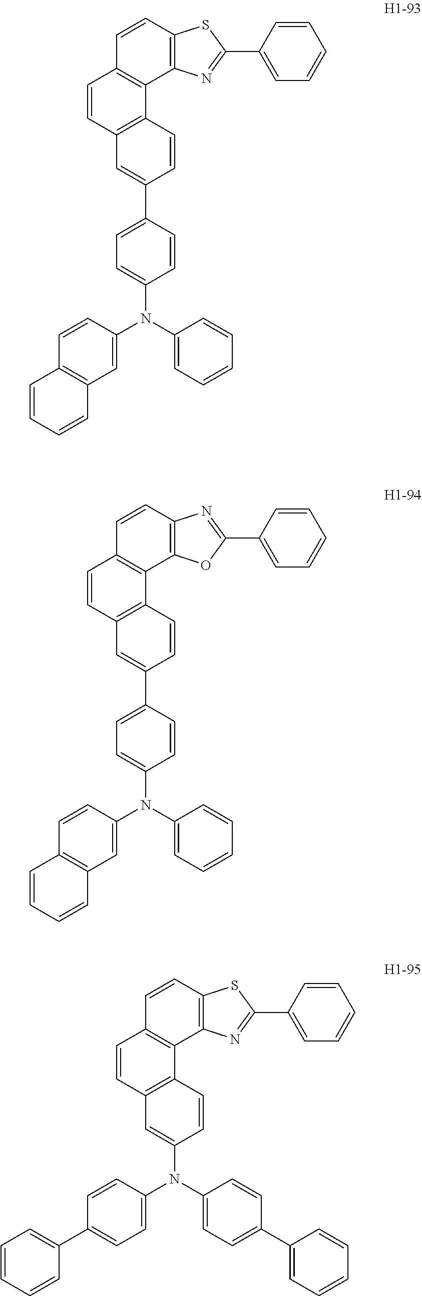 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

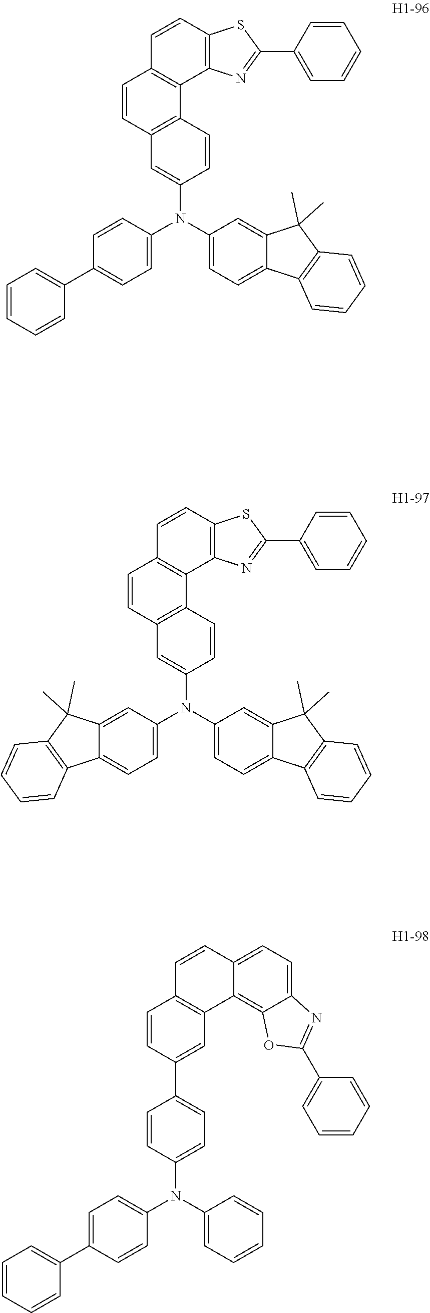 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

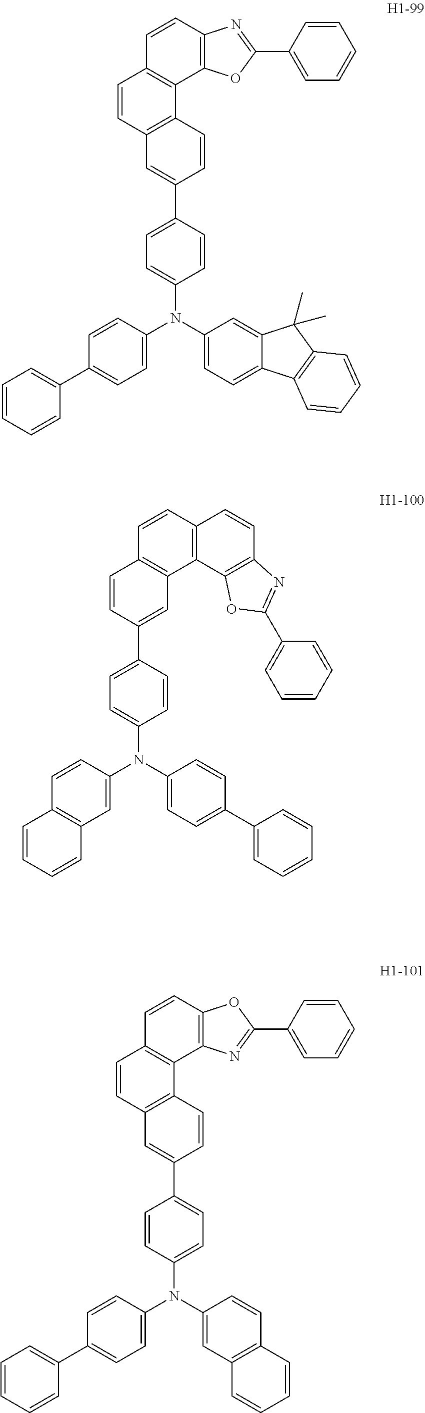 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

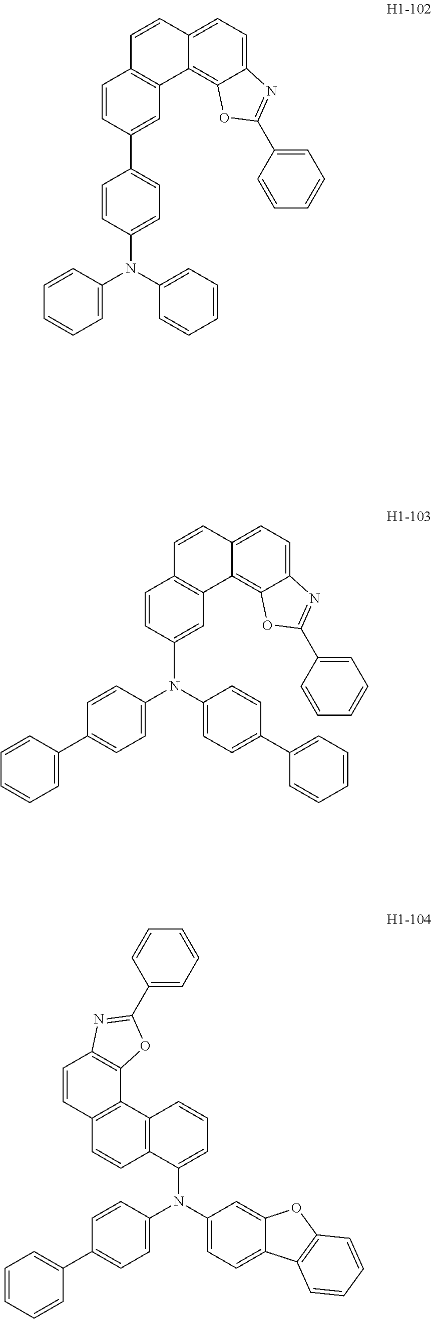 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1]

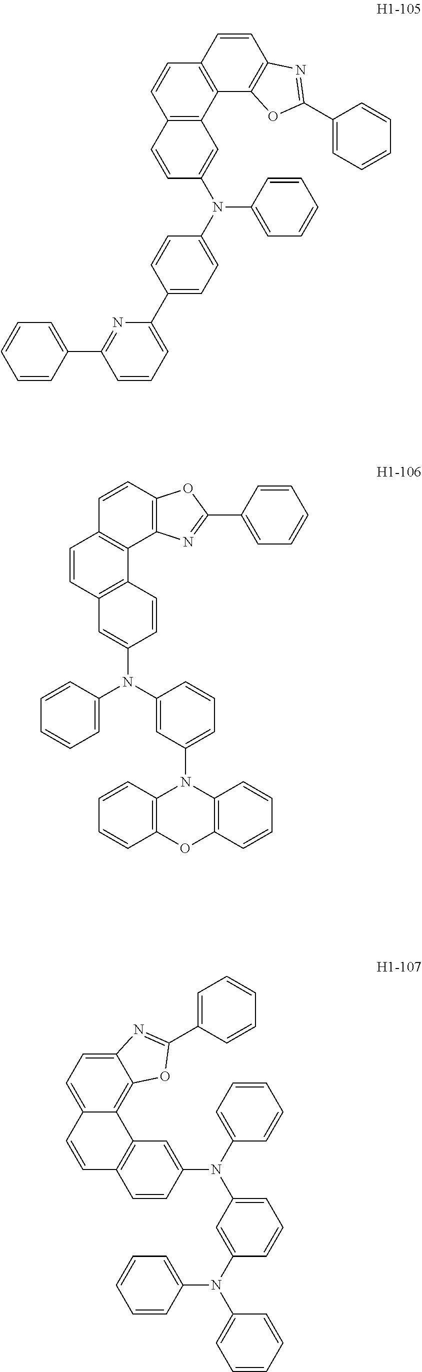 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

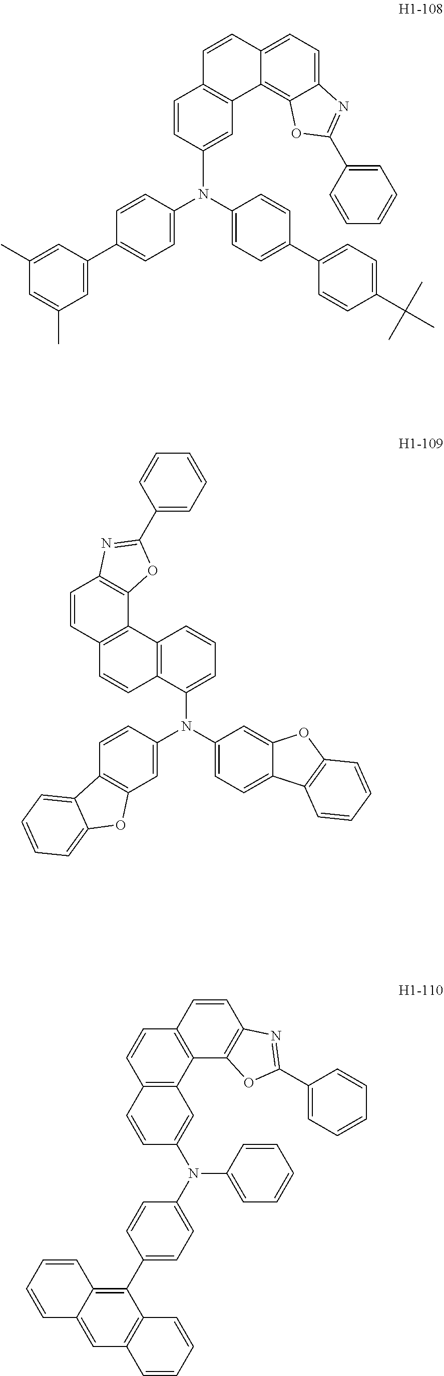 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1]

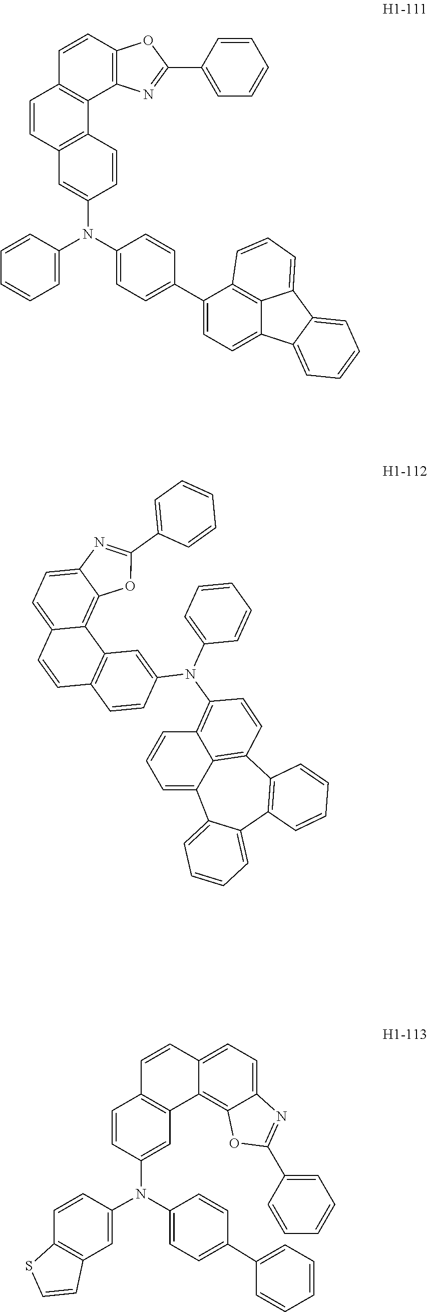

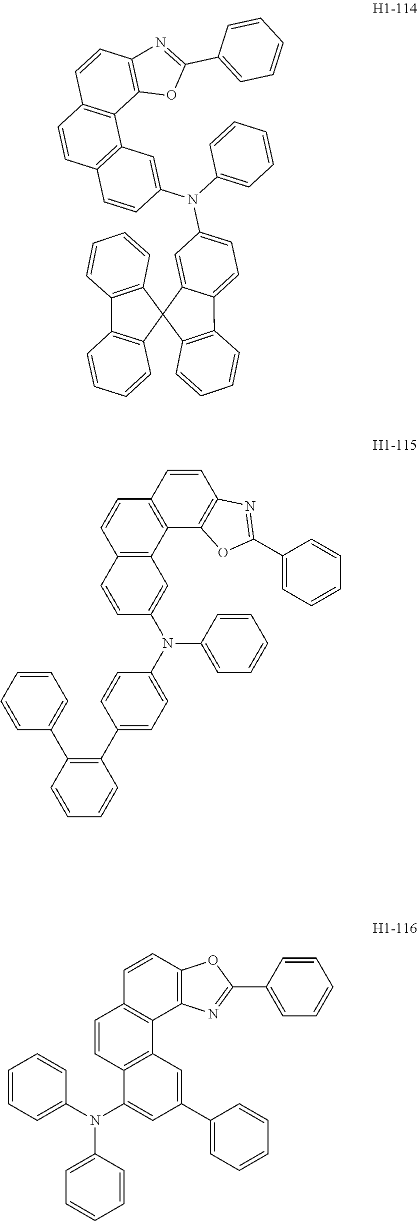 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1]

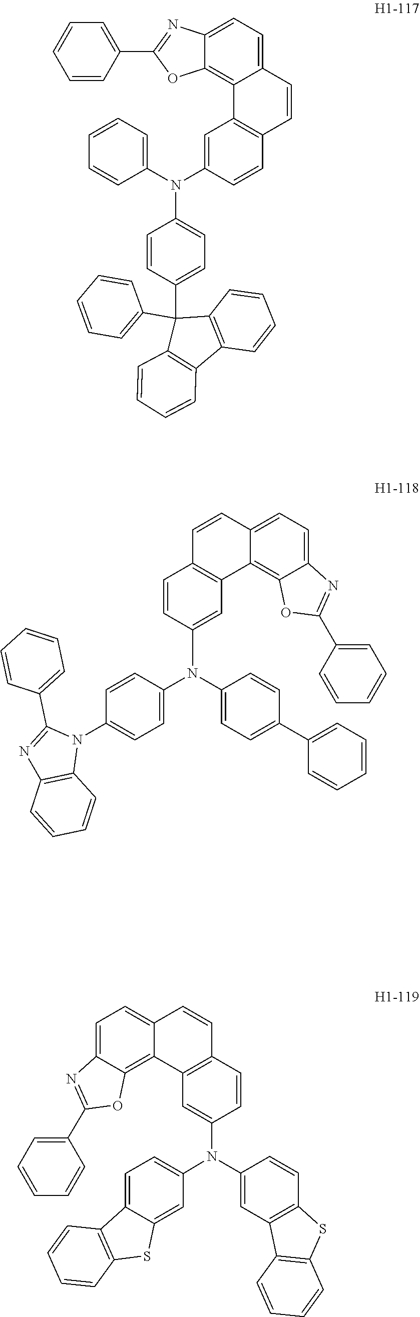 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

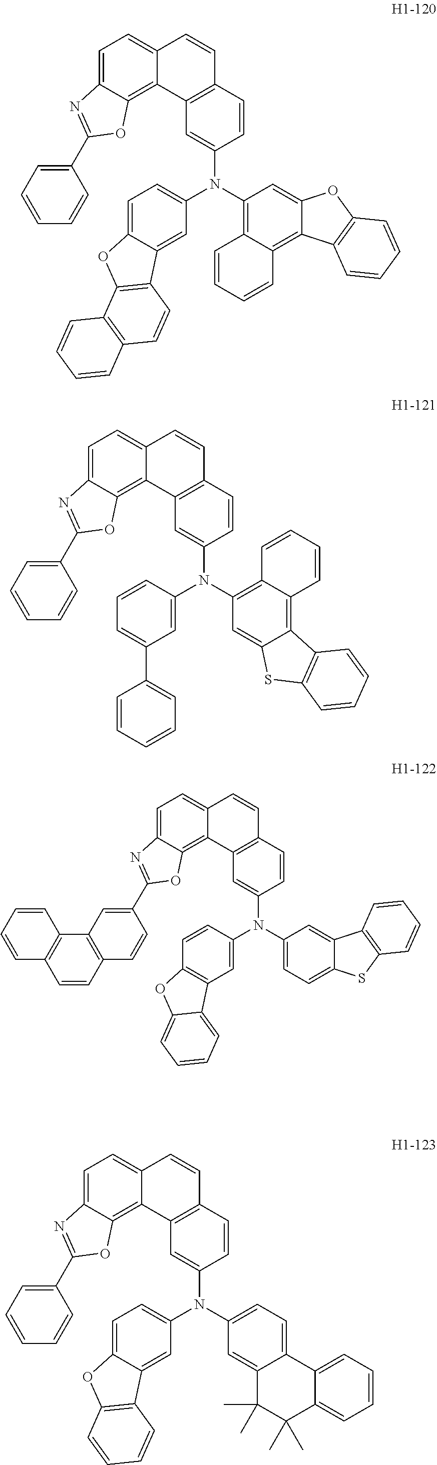 [SMILES: c1ccc(-c2nc3ccc4ccc5ccc(N(c6ccc7oc8c9ccccc9ccc8c7c6)c6cc7oc8ccccc8c7c7ccccc67)cc5c4c3o2)cc1 ; c1ccc(-c2cccc(N(c3ccc4ccc5ccc6nc(-c7ccccc7)oc6c5c4c3)c3cc4sc5ccccc5c4c4ccccc34)c2)cc1 ; c1ccc2c(c1)ccc1ccc(-c3nc4ccc5ccc6ccc(N(c7ccc8oc9ccccc9c8c7)c7ccc8sc9ccccc9c8c7)cc6c5c4o3)cc12 ; CC1(C)c2ccccc2-c2ccc(N(c3ccc4oc5ccccc5c4c3)c3ccc4ccc5ccc6nc(-c7ccccc7)oc6c5c4c3)cc2C1(C)C]

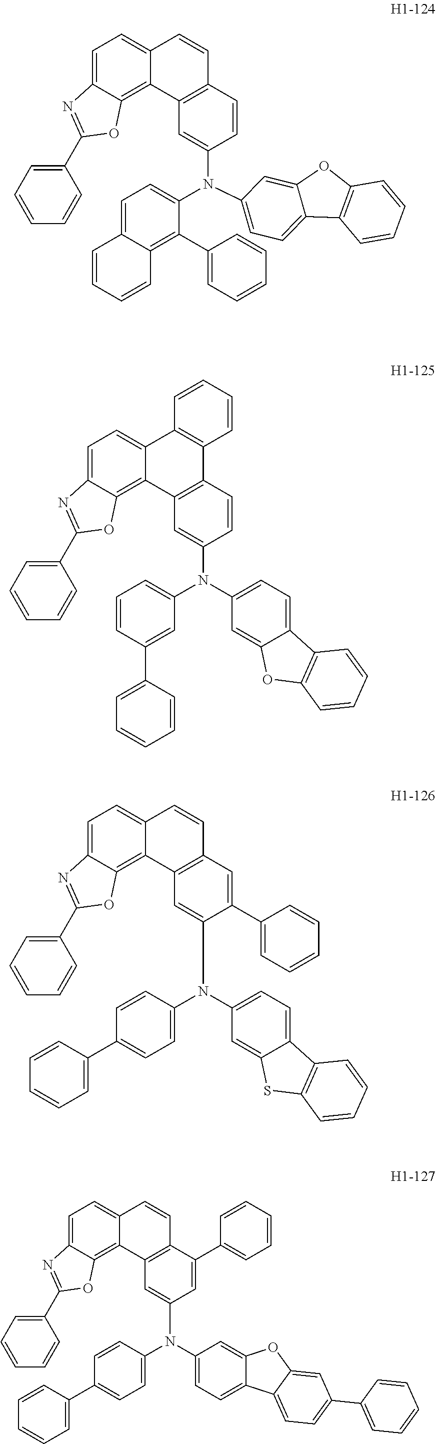 [SMILES: c1ccc(-c2nc3ccc4ccc5ccc(N(c6ccc7c(c6)oc6ccccc67)c6ccc7ccccc7c6-c6ccccc6)cc5c4c3o2)cc1 ; c1ccc(-c2cccc(N(c3ccc4c(c3)oc3ccccc34)c3ccc4c5ccccc5c5ccc6nc(-c7ccccc7)oc6c5c4c3)c2)cc1 ; c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1]

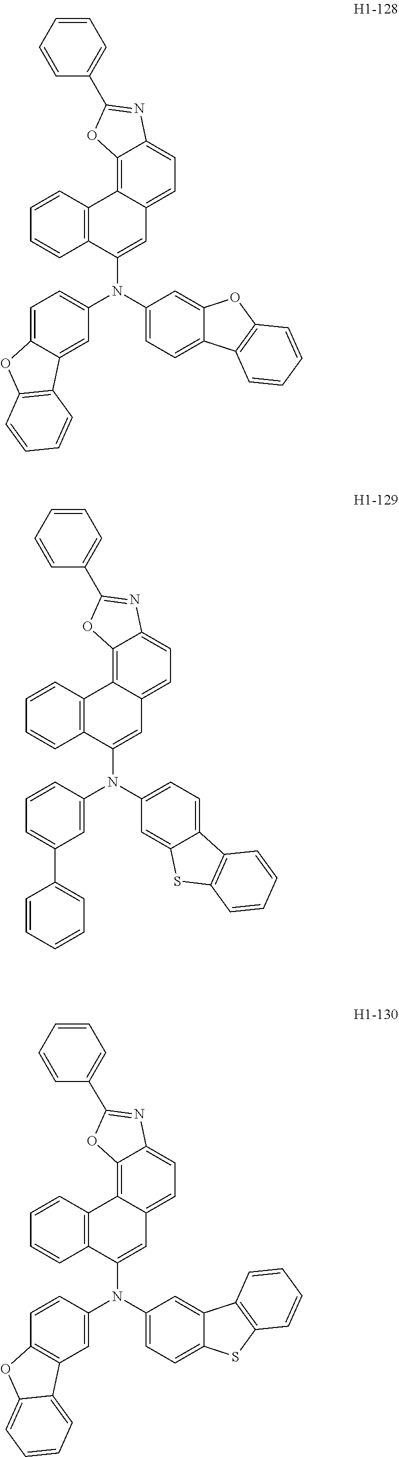 [SMILES: c1ccc(-c2nc3ccc4cc(N(c5ccc6c(c5)oc5ccccc56)c5ccc6oc7ccccc7c6c5)c5ccccc5c4c3o2)cc1 ; c1ccc(-c2cccc(N(c3ccc4c(c3)sc3ccccc34)c3cc4ccc5nc(-c6ccccc6)oc5c4c4ccccc34)c2)cc1 ; c1ccc(-c2nc3ccc4cc(N(c5ccc6oc7ccccc7c6c5)c5ccc6sc7ccccc7c6c5)c5ccccc5c4c3o2)cc1]

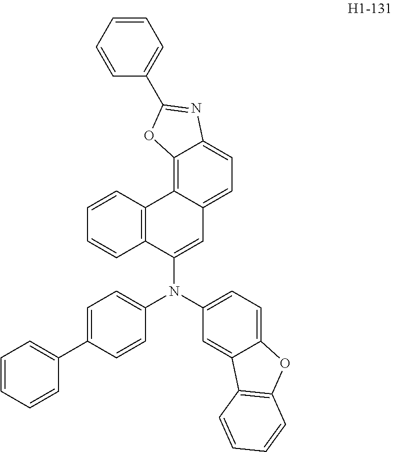 [SMILES: c1ccc(-c2ccc(N(c3ccc4oc5ccccc5c4c3)c3cc4ccc5nc(-c6ccccc6)oc5c4c4ccccc34)cc2)cc1]

The host compound of formula 2 according to the present disclosure may be prepared by a synthetic method known to a person skilled in the art.

According to other embodiment, the present disclosure provides an organic electroluminescent compound represented by the following formula 1.

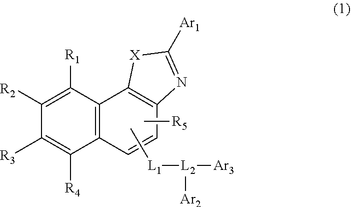

In formula 1,
X represents —O— or —S—;
R₁ to R₅ each independently represent, hydrogen, deuterium, halogen, cyano, a substituted or unsubstituted (C1-C30)alkyl, a substituted or unsubstituted (03-C30)cycloalkyl, a substituted or unsubstituted (C6-C30)aryl, a substituted or unsubstituted (3- to- 30-membered)heteroaryl, a substituted or unsubstituted (C1-C30)alkoxy, or —NRₐR_{b};
Rₐ and R_{b} each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to- 30-membered)heteroaryl;
L₁ represents a single bond or a substituted or unsubstituted (C6-C30)arylene;
L₂ represents a substituted or unsubstituted nitrogen-containing (3- to- 30-membered)heteroarylene; and
Ar₁ to Ar₃ each independently represent, a substituted or unsubstituted (C6-C30)aryl or a substituted or unsubstituted (3- to- 30-membered)heteroaryl; provided that Ar₂ and Ar₃ are different from each other.

Hereinafter, an organic electroluminescent device to which the aforementioned plurality of host materials and/or organic electroluminescent compound is (are) applied, will be described.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host compound represented by Formula 1 and at least one second host compound represented by Formula 2. Wherein, the weight ratio of the first host compound to the second host compound may be in the range of about 1:99 to about 99:1, preferably about 10:90 to about 90:10, more preferably about 30:70 to about 70:30, more preferably about 40:60 to about 60:40, even more preferably about 50:50 in the light-emitting layer.

For example, the organic electroluminescent material of the present disclosure comprises at least one of compounds C-1 to C-74, which is(are) a first host compound, and at least one of compounds H1-1 to H1-131, which is(are) a second host compound. The plurality of host materials may be included in the same organic layer, for example the same light-emitting layer, or may be included in different light-emitting layers.

An organic electroluminescent material according to another embodiment may include at least one compound represented by the Formula 1. The compound of Formula 1 of the present disclosure may preferably be included in the light-emitting layer of an organic electroluminescent device. When included in the light-emitting layer, the compound of Formula 1 may be included as a host, and more specifically, as a phosphorescent red host.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Further, the electron transport layer, the electron injection layer, the electron buffer layer, or the hole blocking layer may contain the azine-based compound as an electron transport material, an electron injection material, an electron buffer material, or a hole blocking material. Further, the organic layer may further comprise at least one metal selected from the group consisting of metals from Group 1, metals from Group 2, transition metals of the 4^{th} period, transition metals of the 5^{th} period, lanthanides, and organic metals of the d-transition elements of the Periodic Table, or at least one complex compound comprising such a metal.

The plurality of host materials and/or the organic electroluminescent compound according to one embodiment may be used as light-emitting materials for a white organic light-emitting device. The white organic light-emitting device has suggested various structures such as a parallel side-by-side arrangement method, a stacking arrangement method, CCM (color conversion material) method, etc., according to the arrangement of R (Red), G (Green), YG (yellowish-green), or B (blue) light-emitting units. In addition, the plurality of host materials and/or the organic electroluminescent compound according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a dual-side emission type according to the kinds of the material forming the first electrode and the second electrode.

A hole injection layer, a hole transport layer, an electron blocking layer, or a combination thereof can be used between the anode and the light-emitting layer. The hole injection layer may be multi-layered in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layered, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layered in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layered, wherein each layer may use a plurality of compounds. Further, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤X≤2), AlOₓ(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Further, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

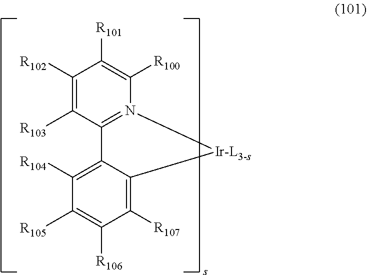

in Formula 101,
L is selected from any one of the following structures 1 to 3;

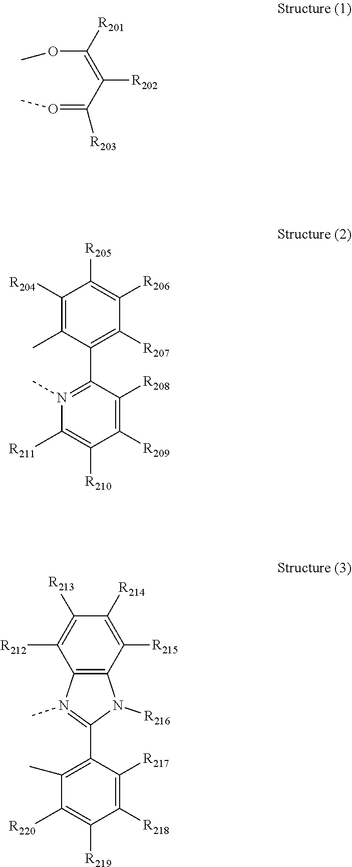

R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to- 30-membered)heteroaryl, or a substituted or unsubstituted (C1-C30)alkoxy; or may be linked to the adjacent substituents to form a ring(s), for example, to form a substituted or unsubstituted ring(s) with pyridine, such as, a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to- 30-membered)heteroaryl, cyano, or a substituted or unsubstituted (C1-C30)alkoxy; or may be linked to the adjacent substituents to form a ring(s), for example, to form a substituted or unsubstituted ring(s) with benzene, such as, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent, hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to the adjacent substituents to form a substituted or unsubstituted ring(s); and
s represents an integer of 1 to 3.

Specifically, the specific examples of the dopant compound include the following, but are not limited thereto.

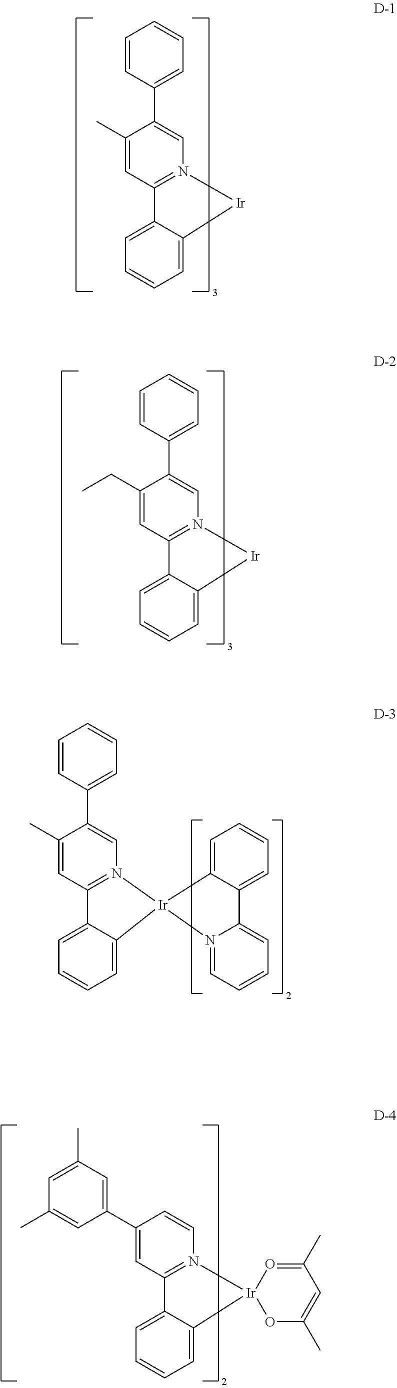

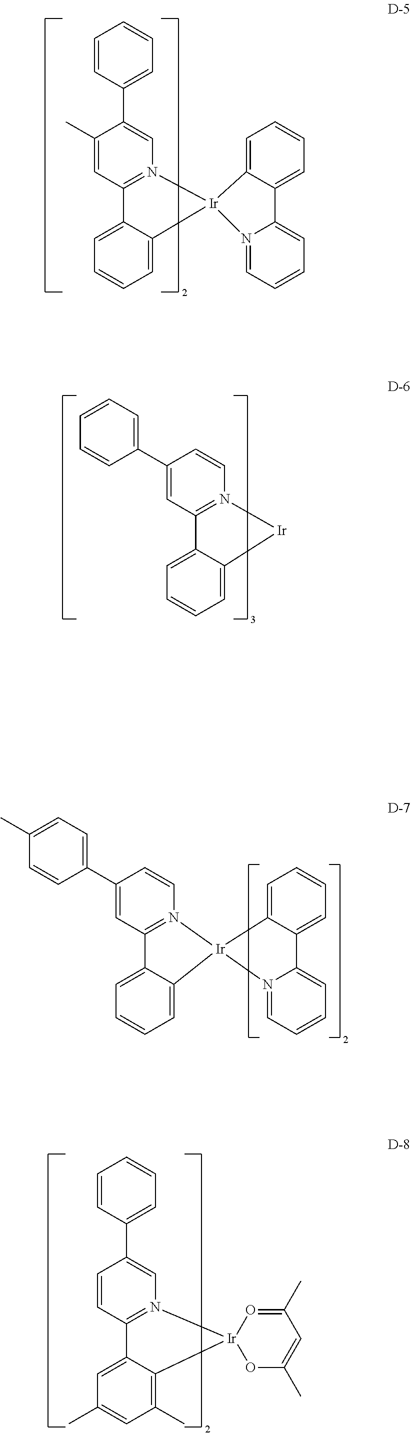

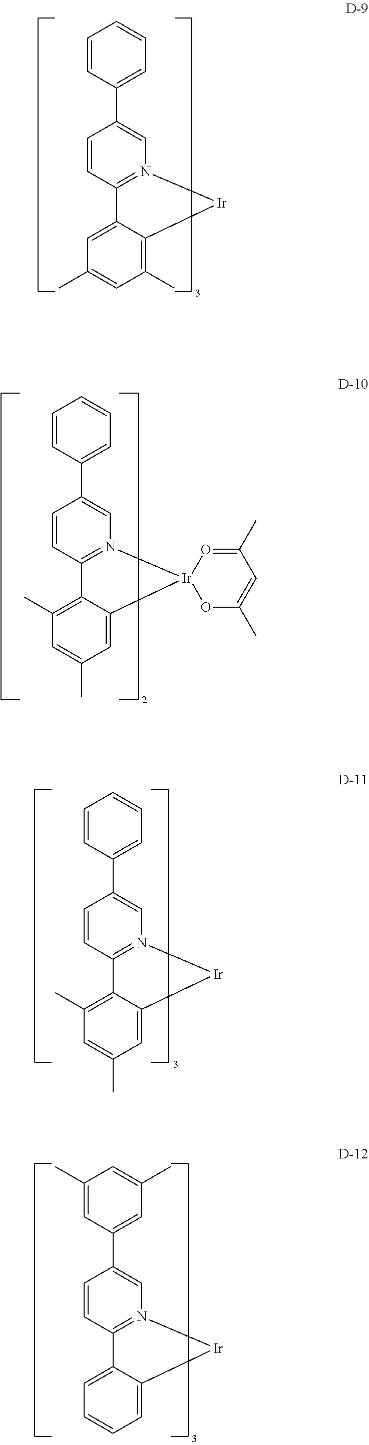

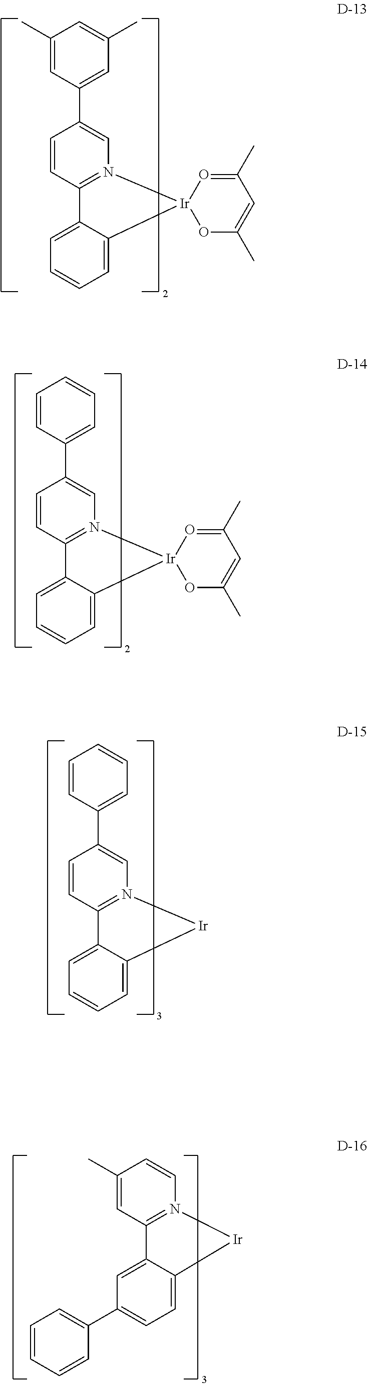

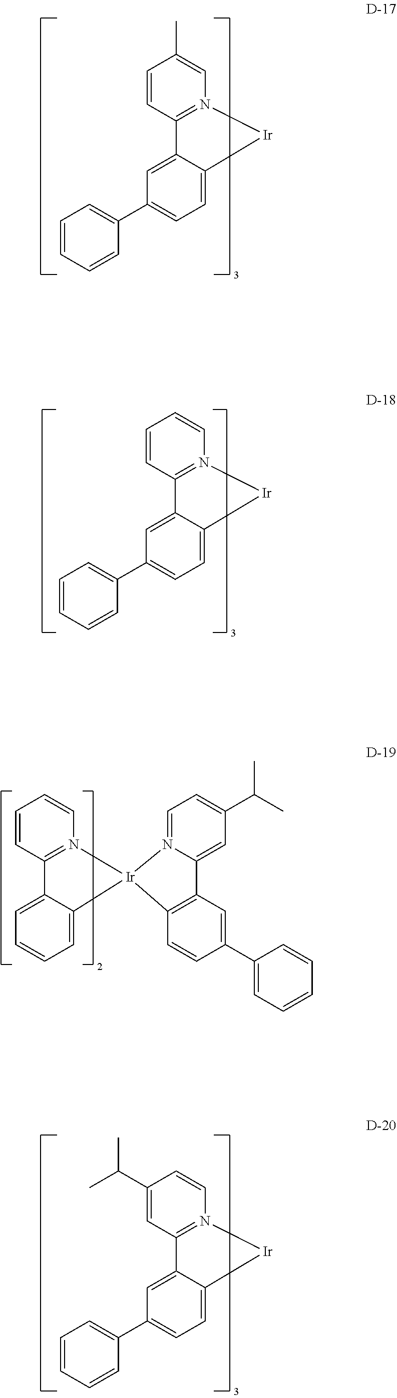

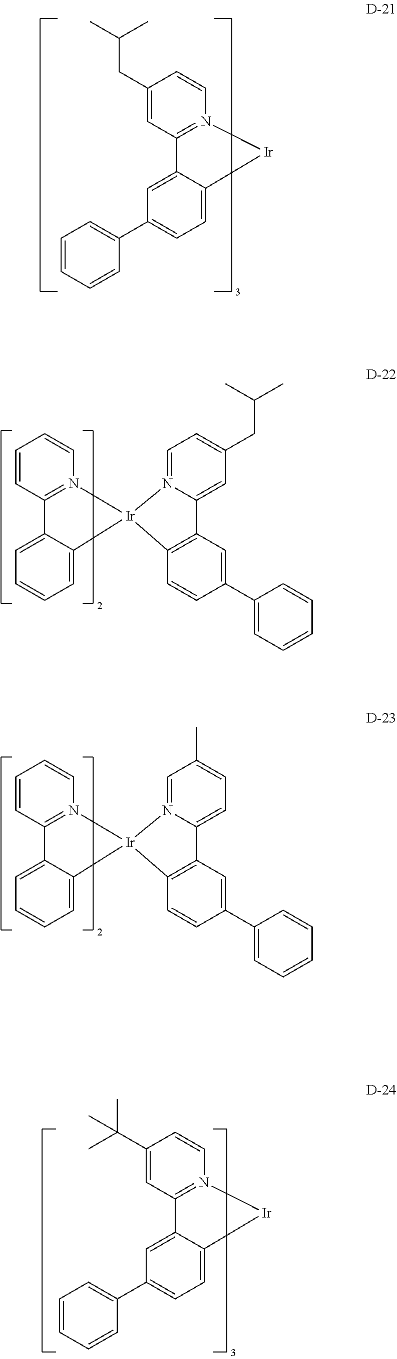

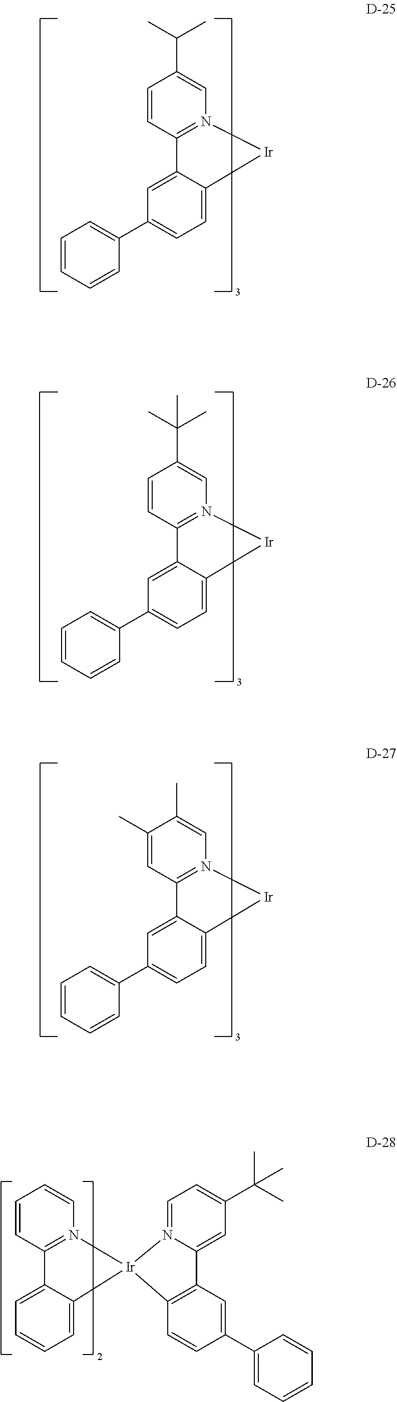

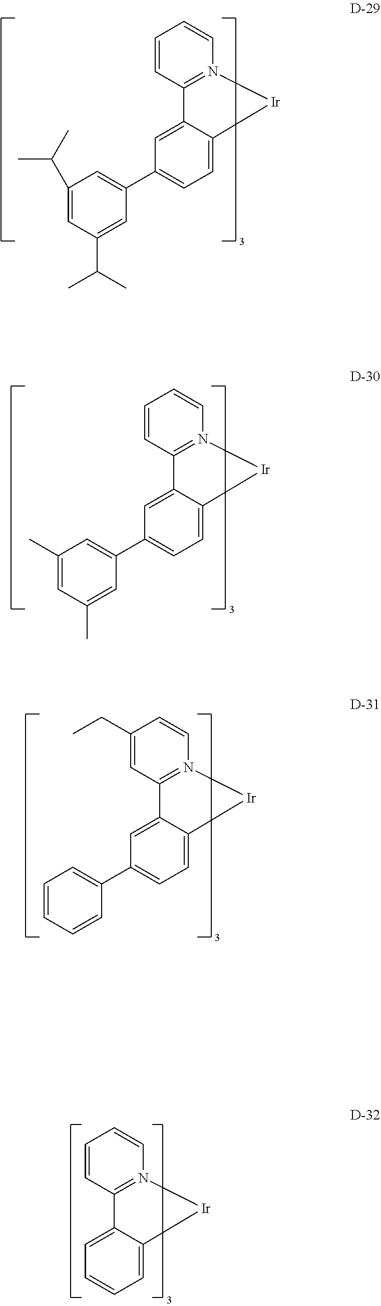

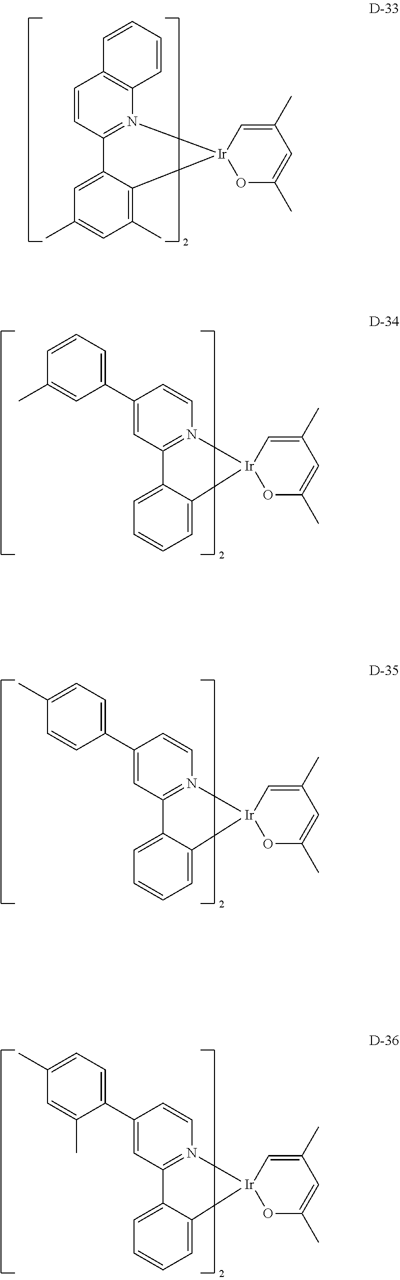

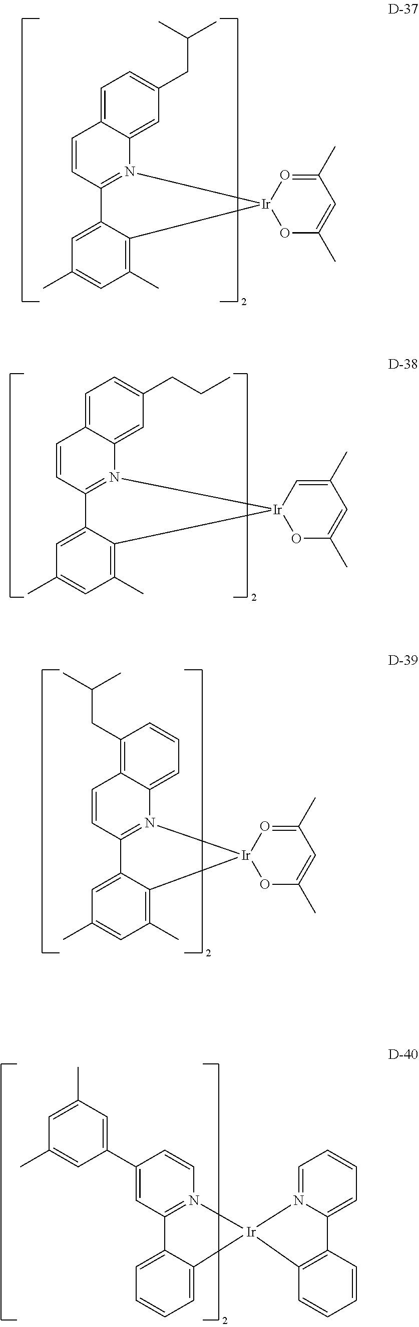

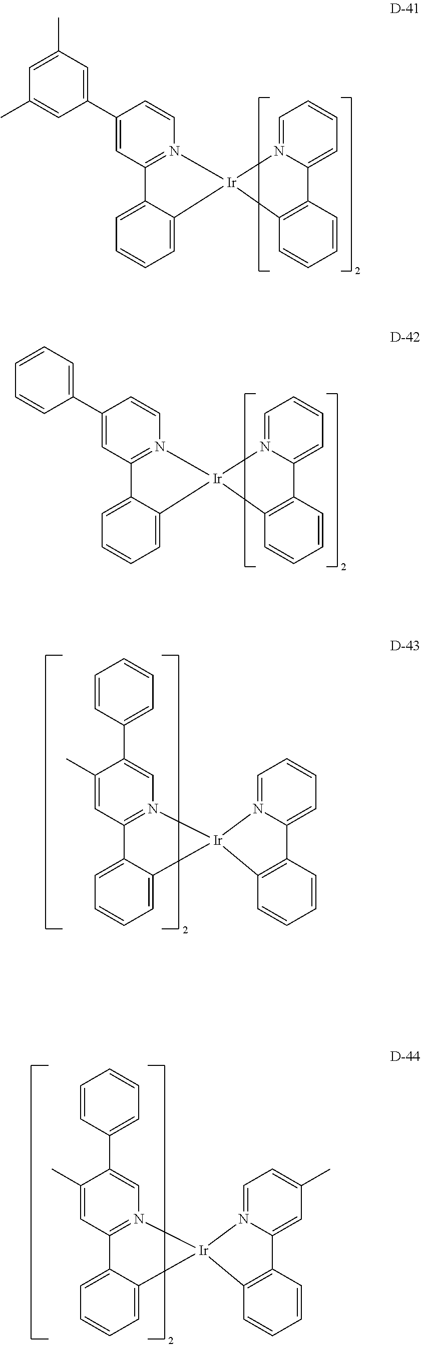

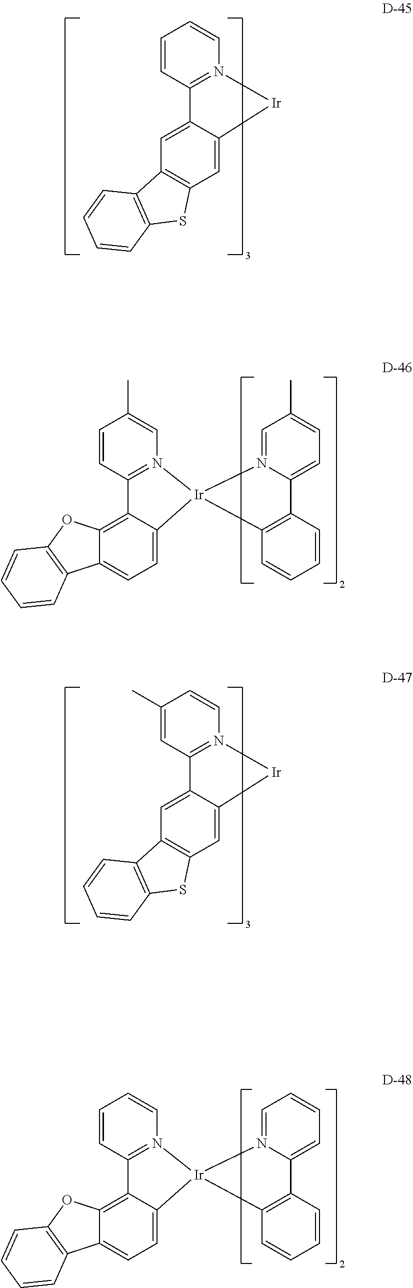

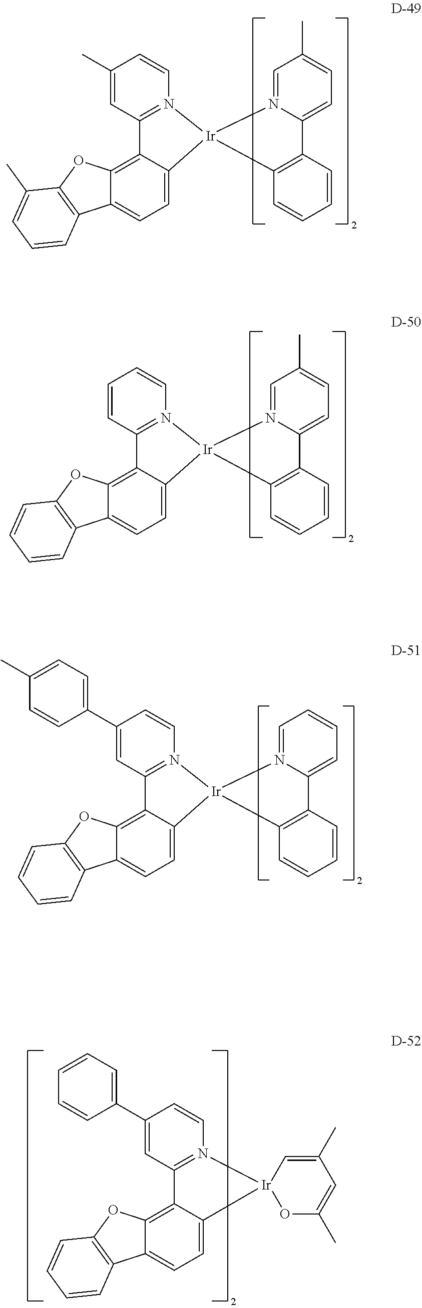

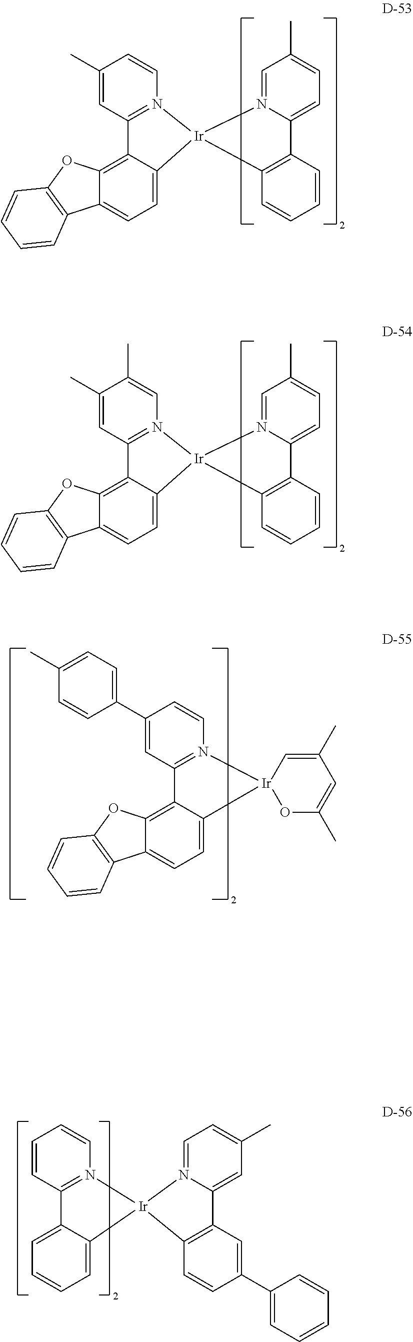

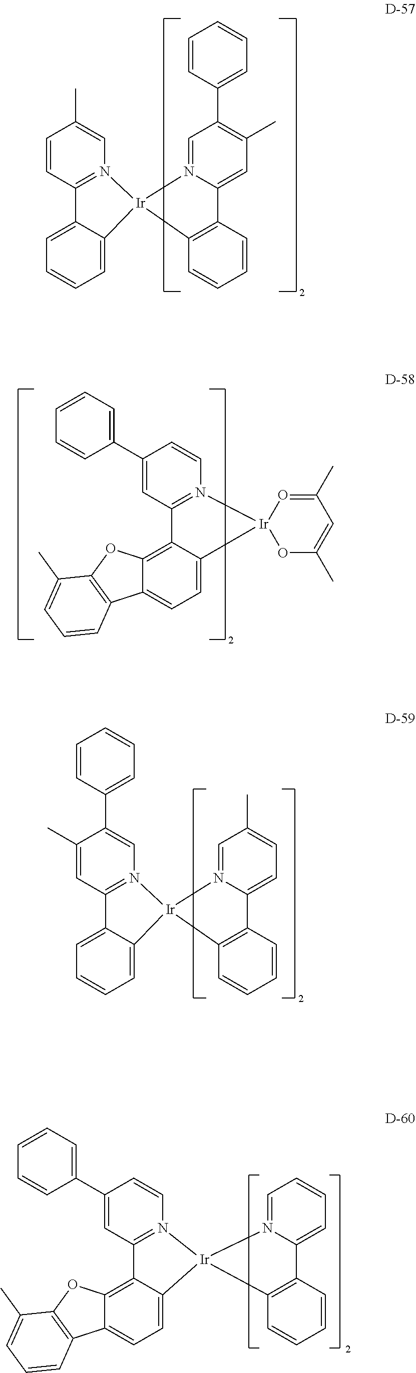

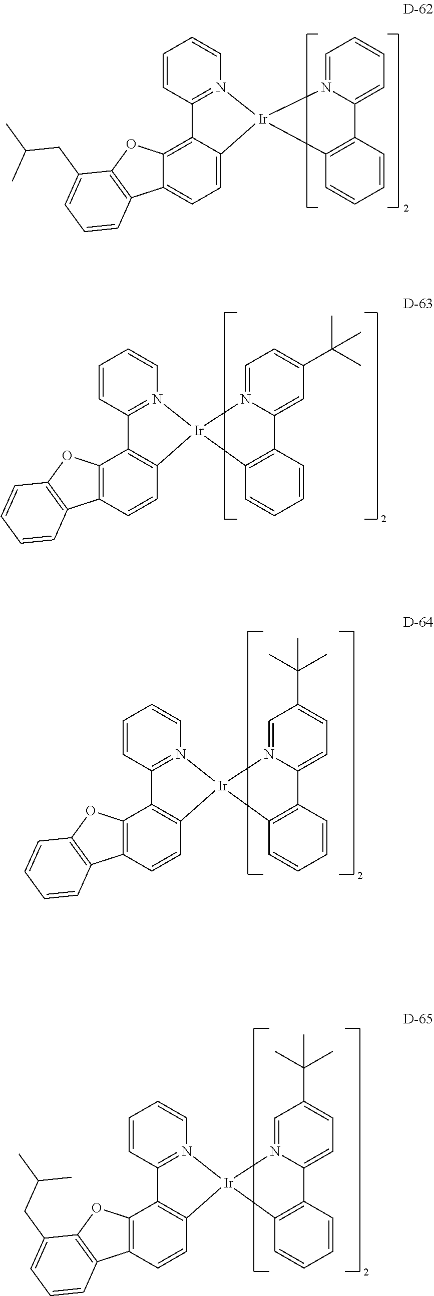

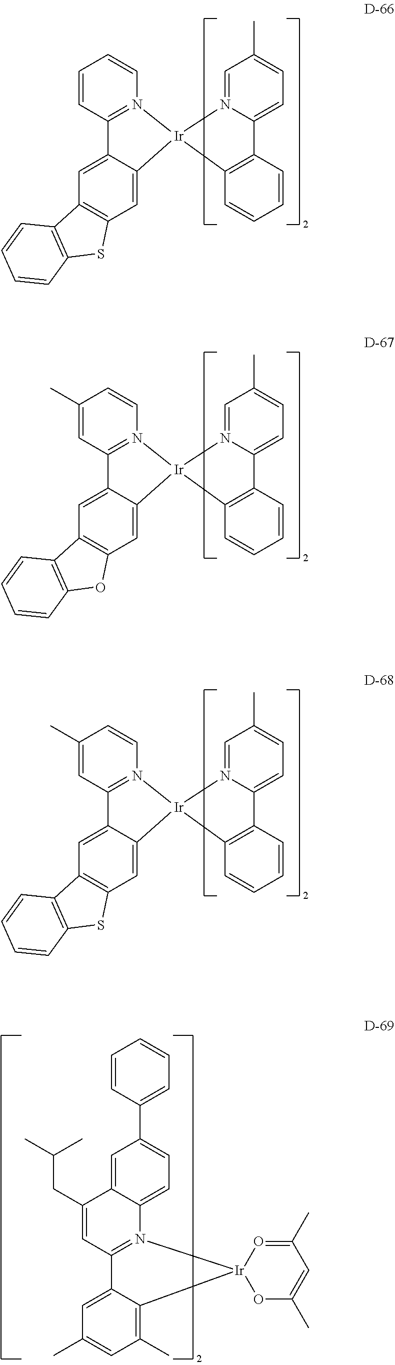

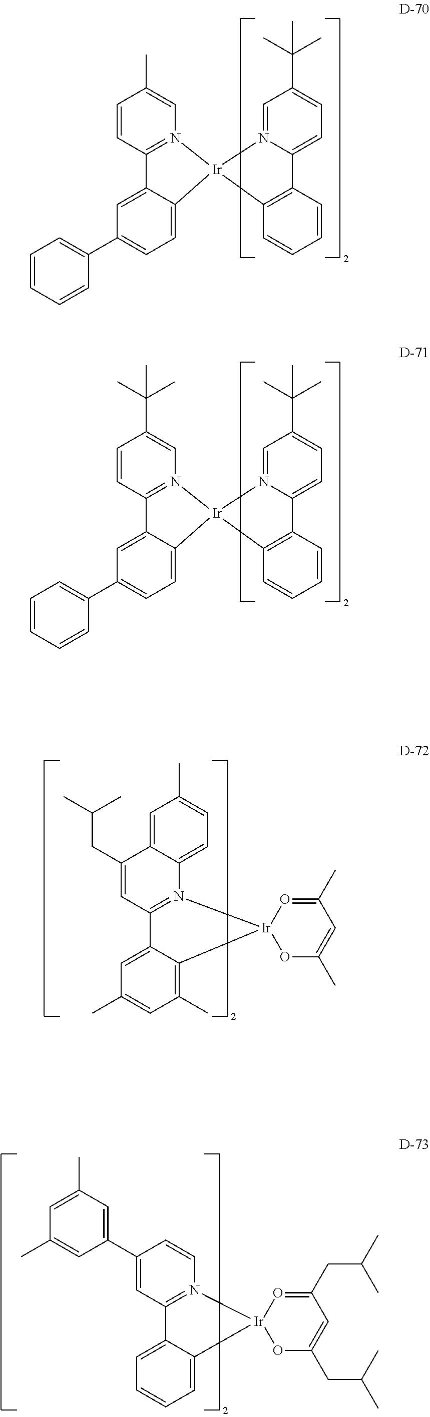

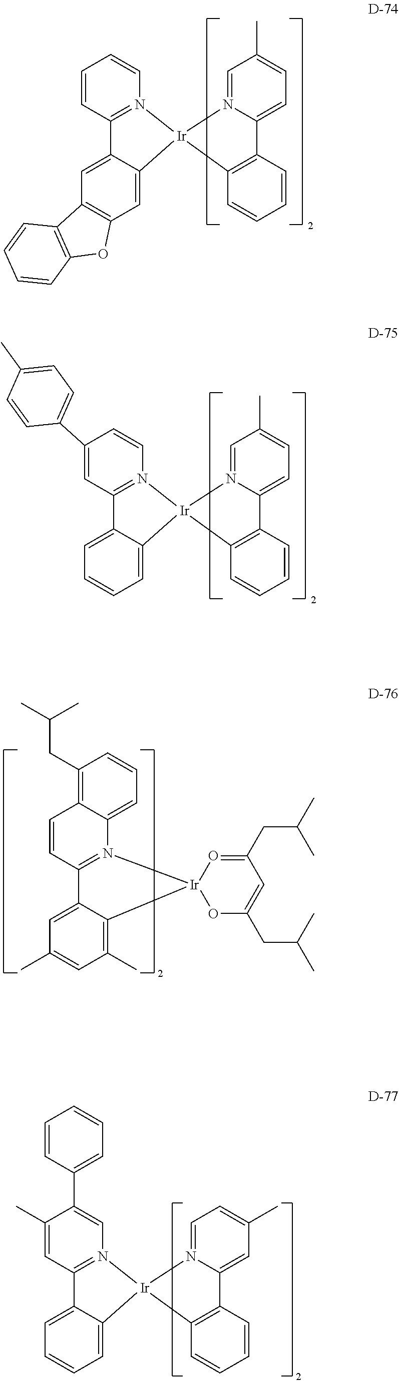

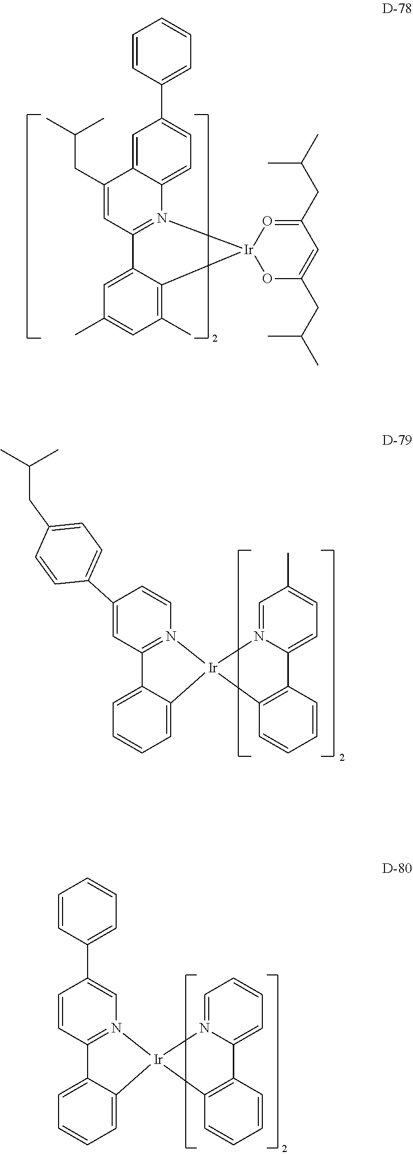

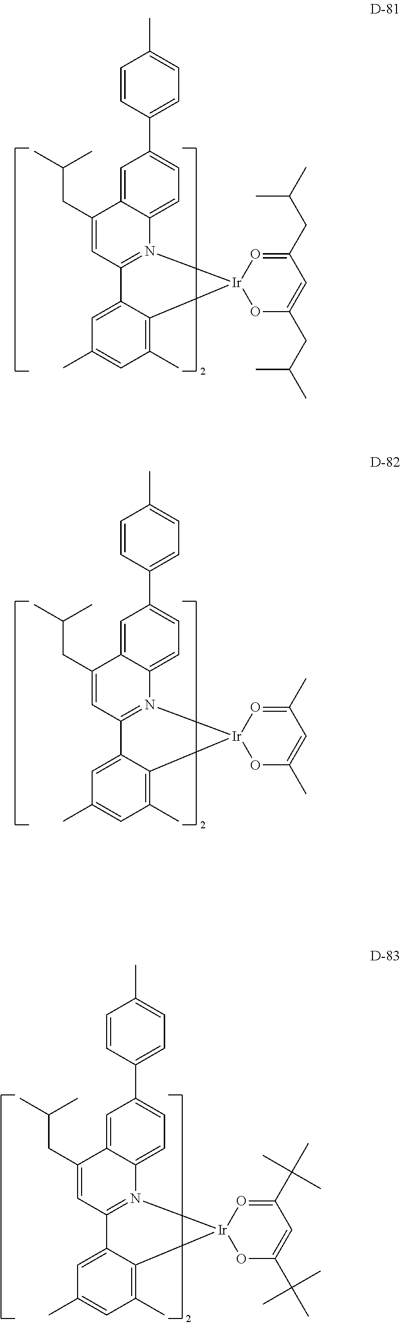

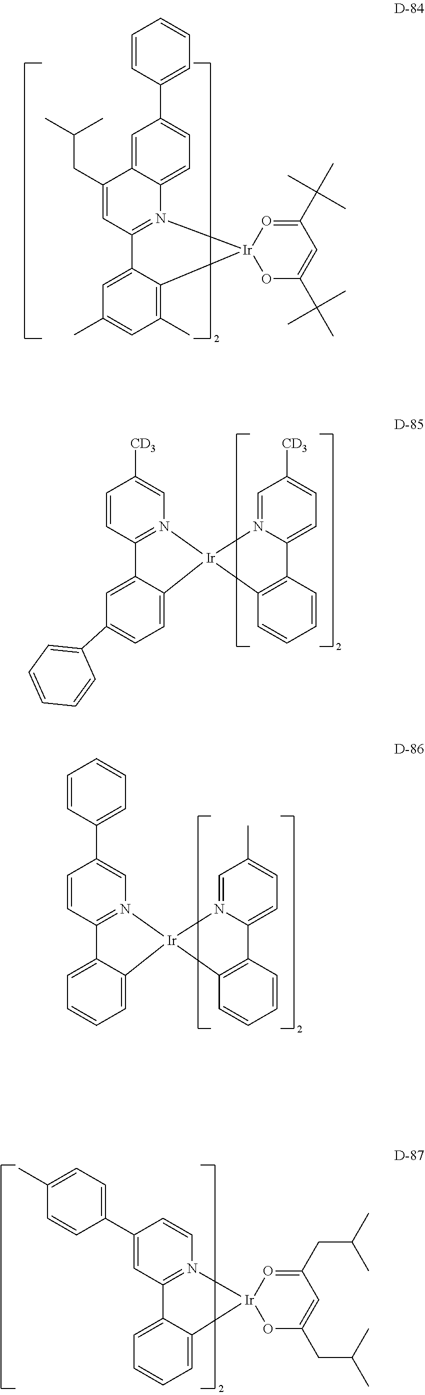

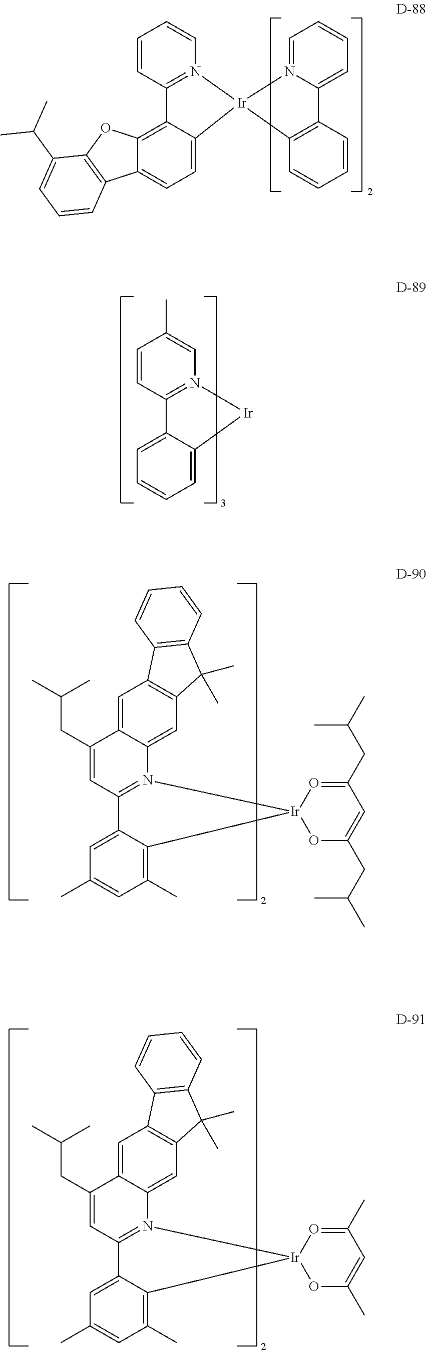

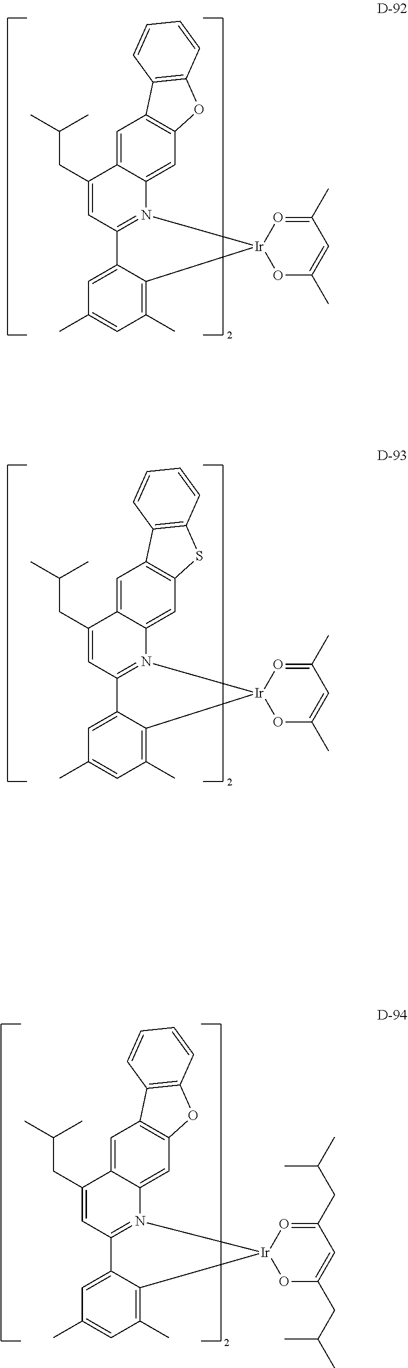

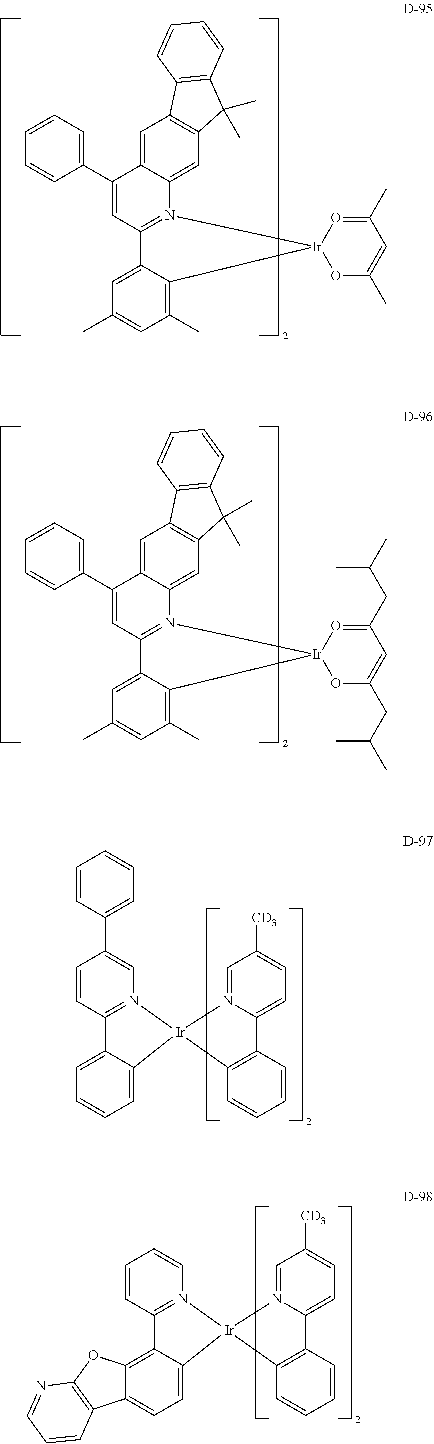

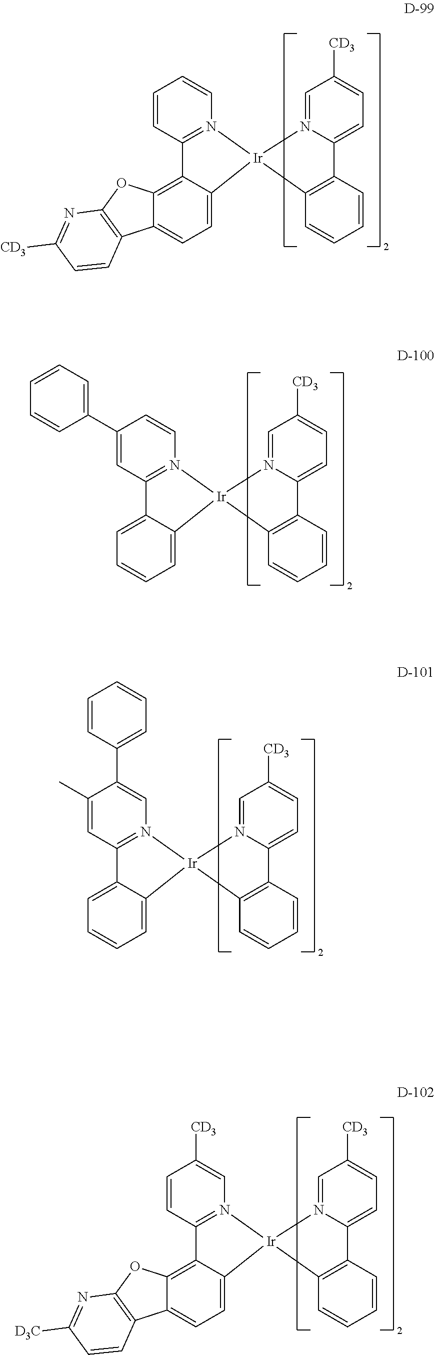

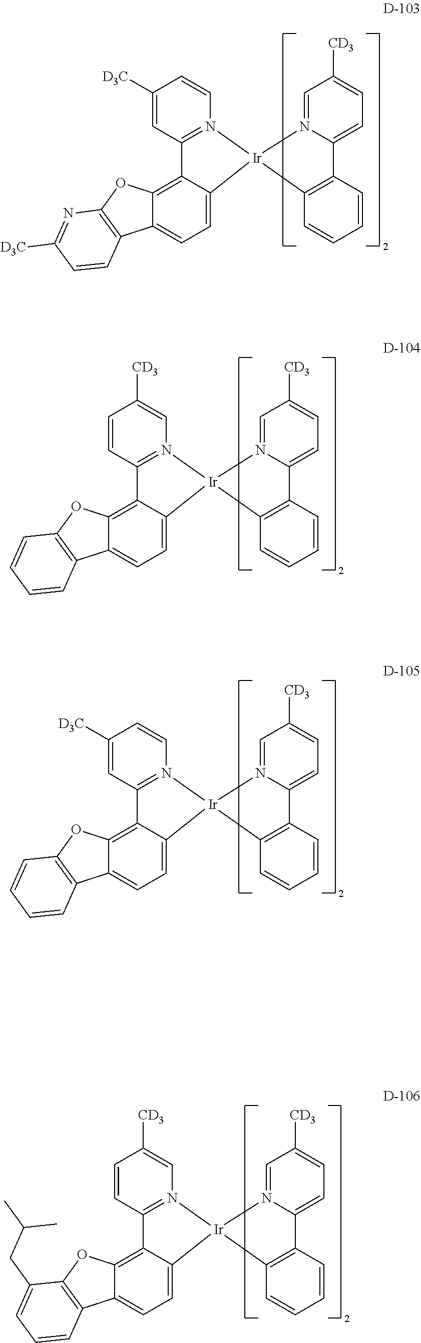

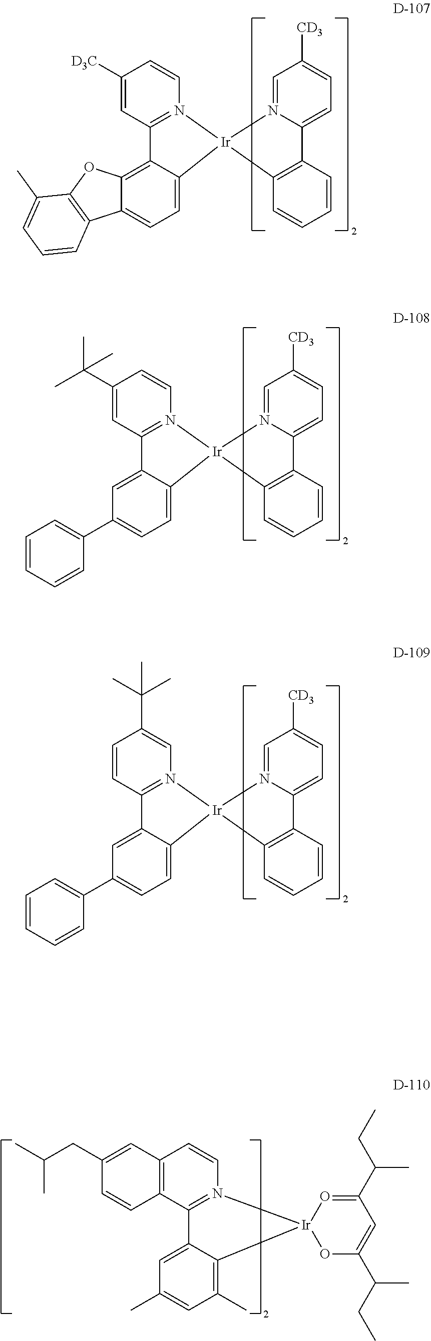

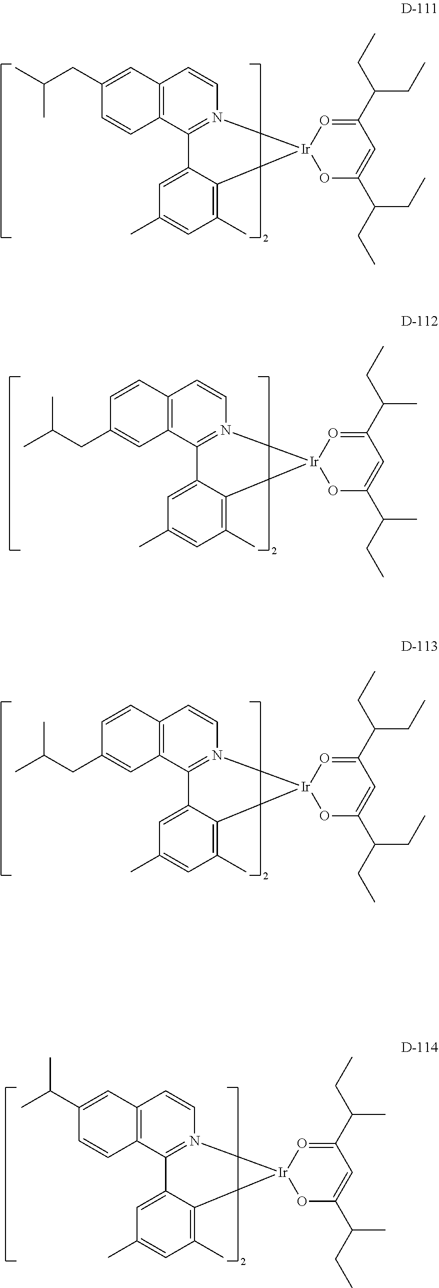

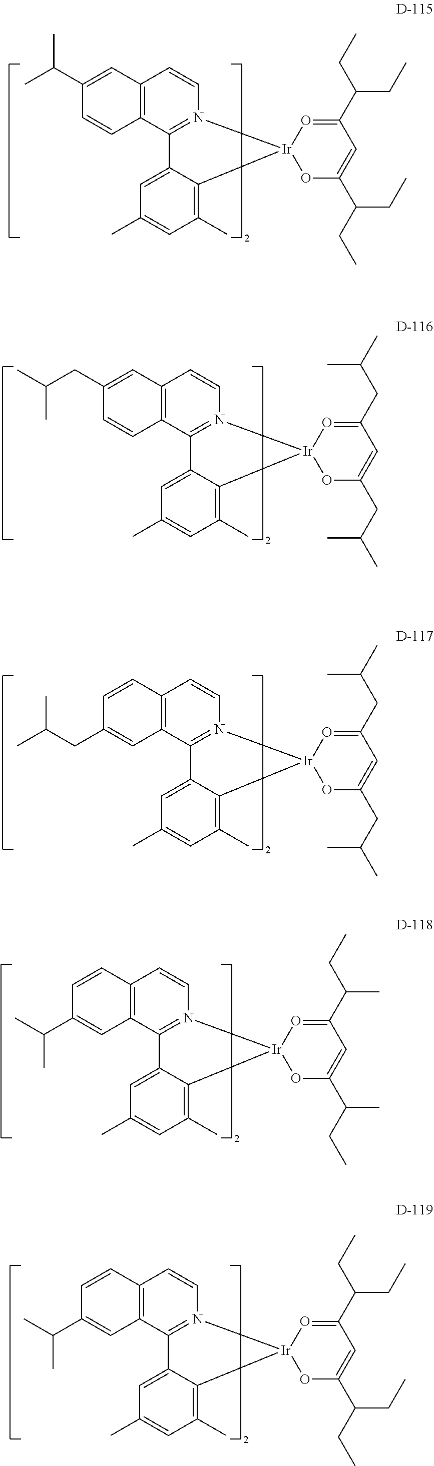

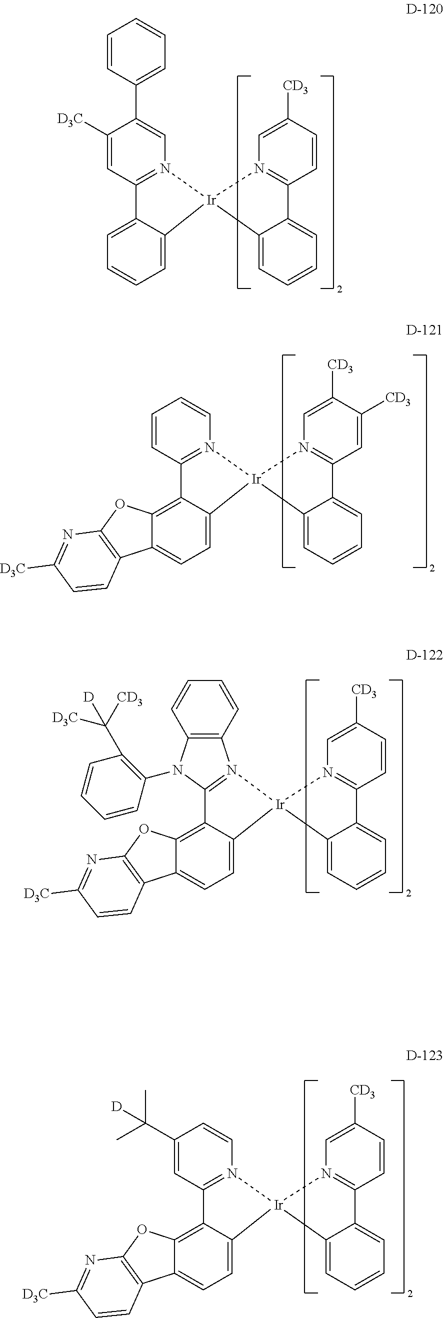

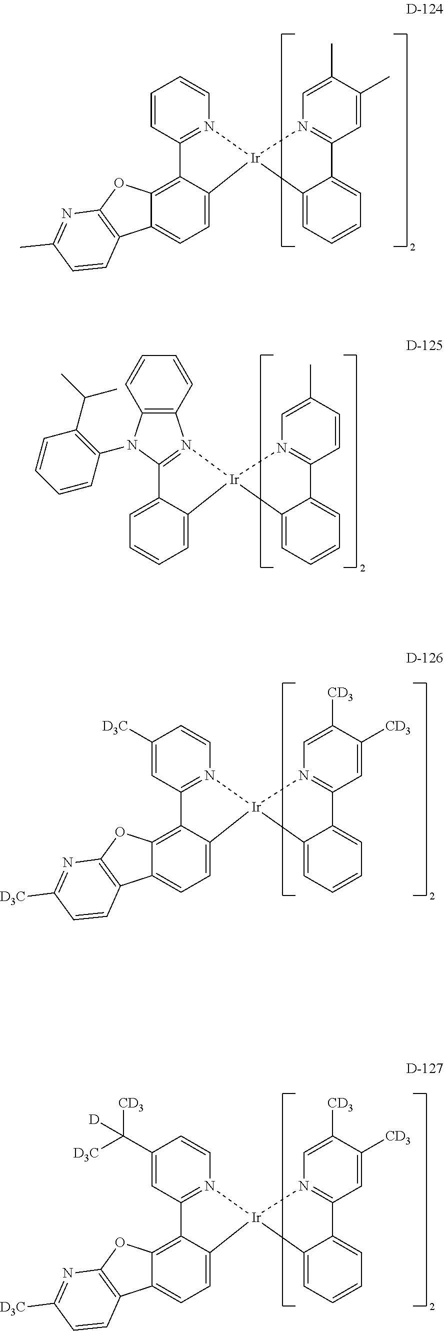

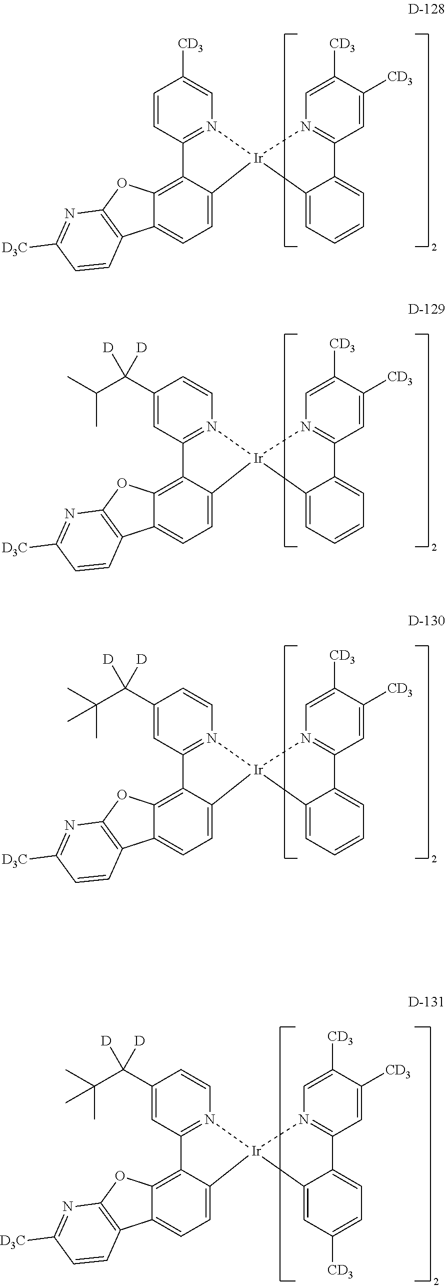

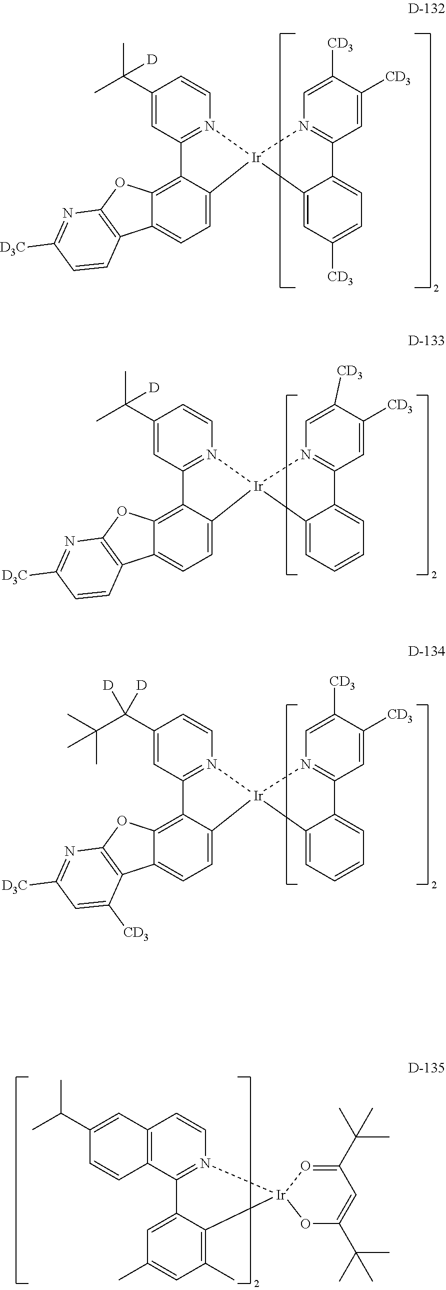

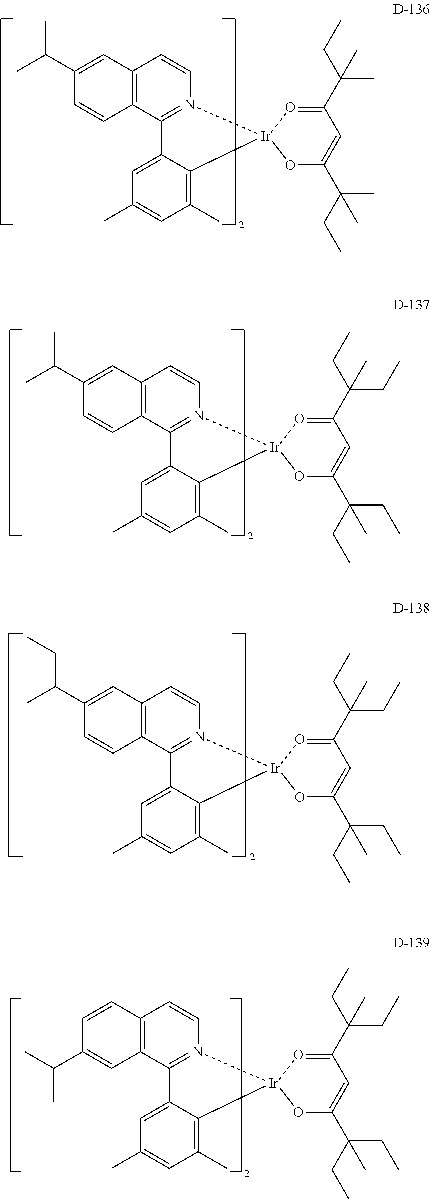

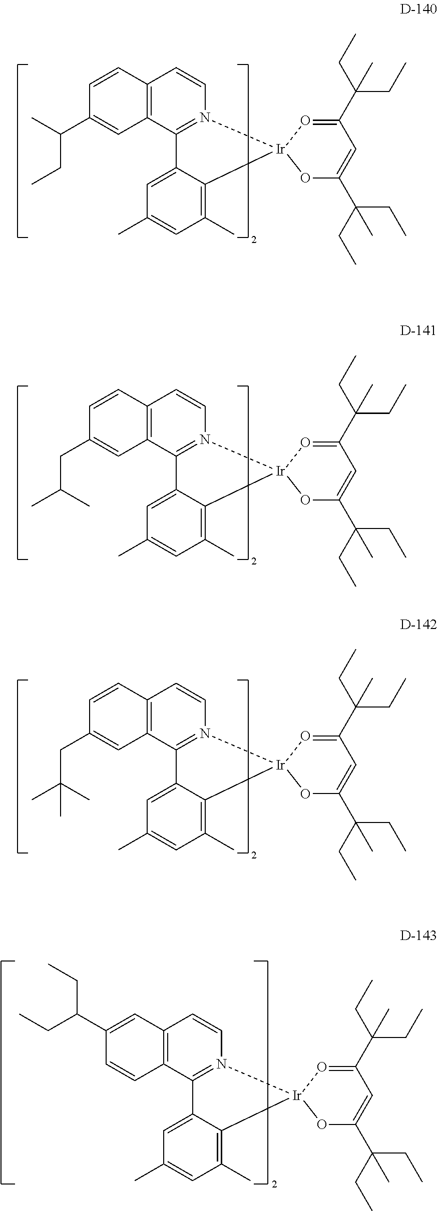

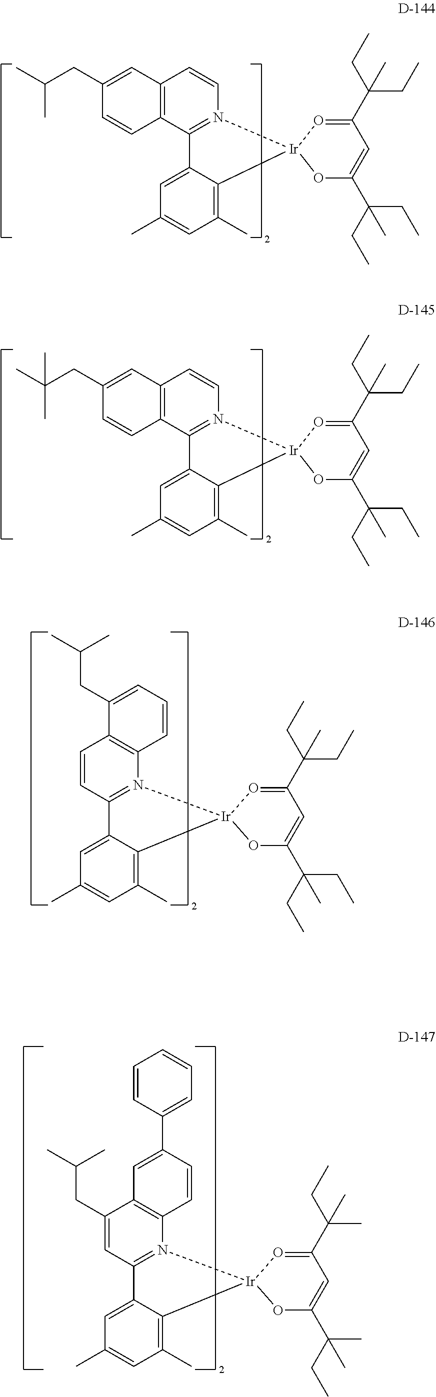

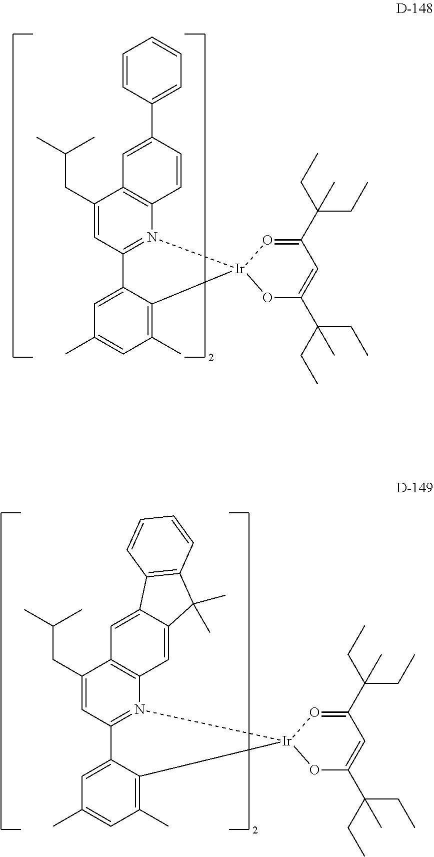

In order to form each layer of the organic electroluminescent device of the present disclosure, dry film-forming methods such as vacuum evaporation, sputtering, plasma, ion plating methods, etc., or wet film-forming methods such as spin coating, dip coating, flow coating methods, etc., can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the first host compound and the second host compound according to one embodiment, the layer can be formed by the above-listed methods, and can often be formed by co-deposition or mixture-deposition. Co-deposition is a mixed deposition method in which two or more materials are put into respective individual crucible sources and a current is applied to both cells simultaneously to evaporate the materials and to perform mixed deposition; and the mixed deposition is a method in which two or more materials are mixed in one crucible source before deposition, and then a current is applied to one cell to evaporate the materials.

According to one embodiment, when the first host compound and the second host compound exist in the same layer or different layers in the organic electroluminescent device, the layers by the two host compounds may be separately formed. For example, after depositing the first host compound, a second host compound may be deposited.

According to one embodiment, the present disclosure can provide display devices comprising a compound represented by Formula 1, or a plurality of host materials comprising a first host compound represented by Formula 1 and a second host compound represented by Formula 2. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the organic electroluminescent compounds according to the present disclosure will be explained with reference to the synthesis method of a representative compound or intermediate compound in order to understand the present disclosure in detail.

### [Example 1] Preparation of Compound C-1

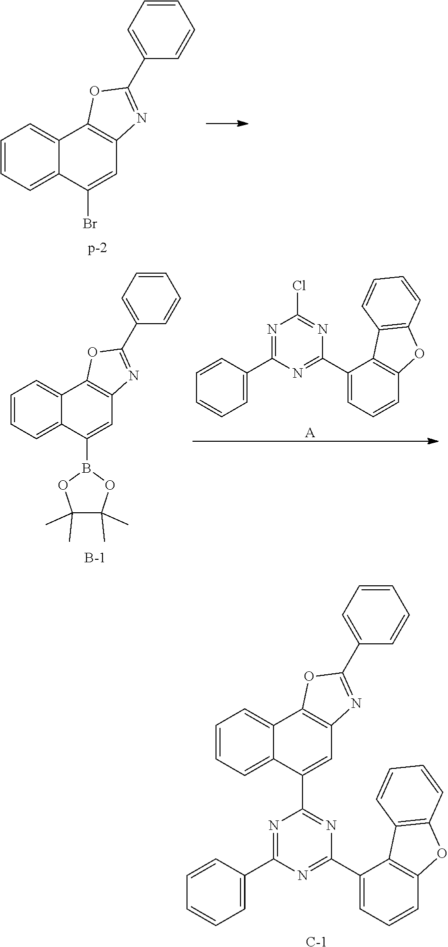

### 1) Synthesis of Compound B-1

Compound p-2 (30 g, 92.6 mmol), potassium acetate (KOAc) (18 g, 185.2 mmol), Pd(PPh₃)₂Cl₂ (6.5 g, 9.2 mmol), 4,4,4′,4′,5,5,5′,5′-octamethyl-2,2′-bi(1,3,2-dioxaborolane) (36 g, 138.9 mmol), and 1 L of dioxane were added to a flask and dissolved, and then reflux at 130° C. for 2 hours. After completion of the reaction, the organic layer was extracted with ethyl acetate, the residual moisture was removed using magnesium sulfate, followed by drying and then separated using column chromatography to obtain Compound B-1 (23 g, yield: 67%).

### 2) Synthesis of Compound C-1

Compound B-1 (20 g, 53.9 mmol), Compound A (23 g, 64.6 mmol), potassium carbonate (K₂CO₃) (15 g, 108 mmol), Pd(PPh₃)₄ (3.2 g, 2.6 mmol), 400 mL of THF, and 80 mL of H₂O were added to a flask and dissolved, and then reflux at 120° C. for 5 hours. After completion of the reaction, the organic layer was extracted with ethyl acetate, the residual moisture was removed using magnesium sulfate, followed by drying and then separated using column chromatography to obtain Compound C-1 (18 g, yield: 58%).

| | MW | M.P |
|---|---|---|
| C-1 | 566.62 | 290° C. |

### [Example 2] Preparation of Compound C-2

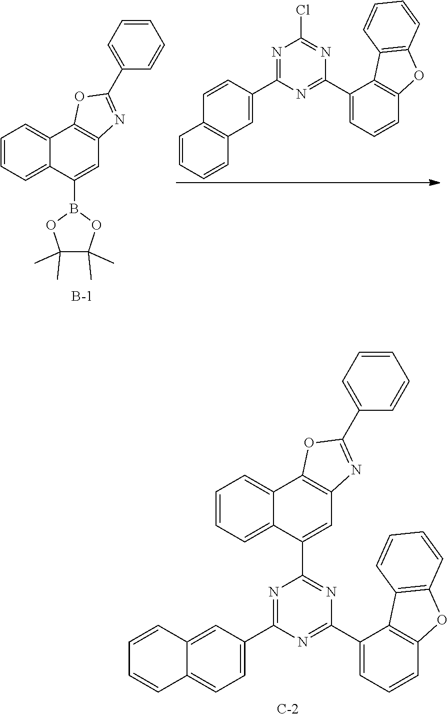

Compound B-1 (20 g, 53.9 mmol), 2-chloro-4-(dibenzo[b,d]furan-1-yl)-6-(naphthalen-2-yl)-1,3,5-triazine (24 g, 59.4 mmol), K₂CO₃ (15 g, 108 mmol), Pd(PPh₃)₄ (3.2 g, 2.6 mmol), 400 mL of THF, and 80 mL of H₂O were added to a flask and dissolved, and then reflux at 120° C. for 5 hours. After completion of the reaction, the organic layer was extracted with ethyl acetate, the residual moisture was removed using magnesium sulfate, followed by drying and then separated using column chromatography to obtain Compound C-2 (3.5 g, yield: 10%).

| | MW | M.P |
|---|---|---|
| C-2 | 616.68 | 296° C. |

### [Example 3] Preparation of Compound C-3

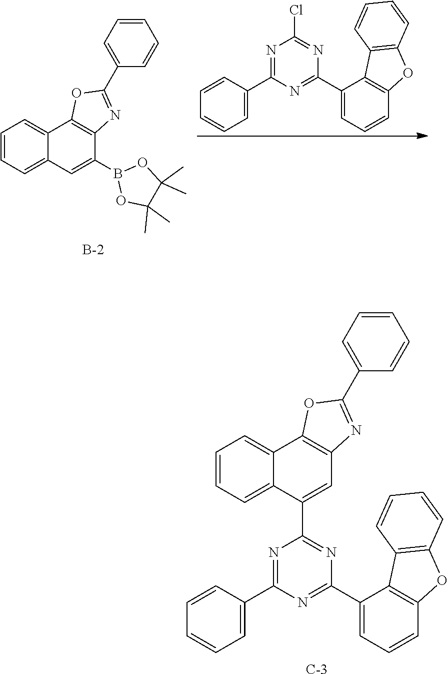

Compound B-2 (7.0 g, 24.2 mmol), 2-chloro-4-(dibenzo[b,d]furan-1-yl)-6-phenyl-1,3,5-triazine (10 g, 29.0 mmol), Pd(PPh₃)₄ (1.4 g, 1.21 mmol), K₂CO₃ (6.7 g, 48.4 mmol), 120 mL of toluene, 60 mL of ethanol (EtOH), and 60 mL of H₂O were added to a flask and dissolved, and then stirred under reflux for 12 hours. After completion of the reaction, it was concentrated under reduced pressure, and the organic layer was extracted with dichloromethane and filtered through a silica gel pad. After concentrating the organic layer, the obtained product was separated using a silica gel column to obtain Compound C-3 (6.2 g, yield: 45.2%).

| | MW | M.P |
|---|---|---|
| C-3 | 566.62 | 280.2° C. |

### [Device Examples 1 to 3] Preparation of OLEDs by Co-Depositing the First Host Compound and the Second Host Compound According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, thereafter, it was stored in isopropanol and used. Next, the ITO substrate was mounted on a substrate holder of a vacuum vapor deposition apparatus. Then, Compound HI-1 was introduced into a cell of the vacuum vapor deposition apparatus, while Compound HT-1 was introduced into another cell. The two materials were evaporated at different rates and Compound HI-1 was deposited in a doping amount of 3 wt % based on the total amount of Compound HI-1 and Compound HT-1 to form a hole injection layer having a thickness of 10 nm. Next, Compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-39 was introduced into another cell as a dopant. The two host materials were evaporated at a rate of 1:1 and the dopant material was evaporated at a different rate, simultaneously, and the dopant was deposited in a doping amount of 3 wt % based on the total amount of the hosts and dopant to form a light-emitting layer having a thickness of 40 nm on the second hole transport layer. Next, compounds ET-1 and EI-1 as electron transport materials were deposited at a weight ratio of 50:50 to form an electron transport layer having a thickness of 35 nm on the light-emitting layer. After depositing Compound EI-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 1] Preparation of an OLED Comprising a Single Host Compound

An OLED was manufactured in the same manner as in Device Example 1, except that the first host compound described in the following Table 1 was used alone as the host of the light-emitting layer.

The driving voltage, luminous efficiency, and the luminous color at a luminance of 1,000 nits and the time taken for luminance to decrease from 100% to 95% at a luminance of 10,000 nits (lifespan: T95) of the OLEDs of Device Examples 1 to 3 and Device Comparative Example 1 produced as described above, are measured, and the results thereof are shown in the following Table 1.

TABLE 1

| | | | Driving | Luminous | Lumi- | |
|---|---|---|---|---|---|---|
| | First | Second | Voltage | Efficiency | nous | Lifespan |
| | Host | Host | (V) | (cd/A) | Color | T95(hr) |
| Device | C-2 | H1-16 | 3.0 | 32.7 | Red | 162 |
| Example 1 | | | | | | |
| Device | C-3 | H1-16 | 3.2 | 35.4 | Red | 126 |
| Example 2 | | | | | | |
| Device | C-1 | H1-16 | 3.0 | 34.6 | Red | 141 |
| Example 3 | | | | | | |
| Device | A-1 | — | 4.0 | 28.0 | Red | 4 |
| Comparative | | | | | | |
| Example 1 | | | | | | |

From Table 1 above, it can be seen that the device example including a specific combination of host materials according to the present disclosure exhibits low driving voltage and/or high luminous efficiency and/or long lifespan characteristics.

The LUMO energy level, HOMO energy level, and triplet energy of Compounds C-1 and C-69, which are represented by Formula 1 according to the present disclosure, and Comparative Compound A-2 were measured, respectively, and are shown in Table 2 below.

TABLE 2

| | 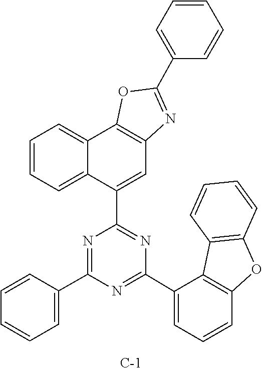 [SMILES: c1ccc(-c2nc(-c3cc4nc(-c5ccccc5)oc4c4ccccc34)nc(-c3cccc4oc5ccccc5c34)n2)cc1] | 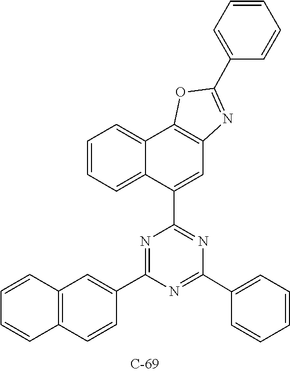 [SMILES: c1ccc(-c2nc(-c3ccc4ccccc4c3)nc(-c3cc4nc(-c5ccccc5)oc4c4ccccc34)n2)cc1] | 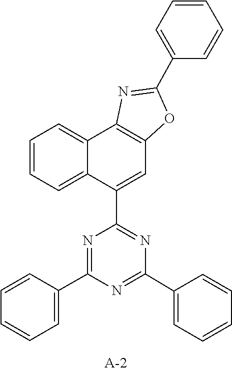 [SMILES: c1ccc(-c2nc(-c3ccccc3)nc(-c3cc4oc(-c5ccccc5)nc4c4ccccc34)n2)cc1] |
|---|---|---|---|
| LUMO (ev) | −1.922 | −1.927 | −2.047 |
| HOMO (ev) | −5.740 | −5.687 | −5.595 |
| Triplet (ev) | 2.531 | 2.493 | 2.246 |

Referring to Table 2 above, Comparative Compound A-2, which has a different basic skeleton from the compound of Formula 1 according to the present disclosure, has a lower triplet energy value compared to Compounds C-1 and C-69. Therefore, it can be expected that the efficiency of the device will decrease when using it as an organic electroluminescent material.

The compounds used in Device Examples and Device Comparative Example are specifically shown in the following Table 3.

TABLE 3

| | |
|---|---|
| Hole Injection Layer/Hole Transport Layer | 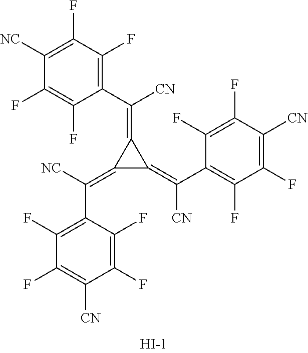 |
| | 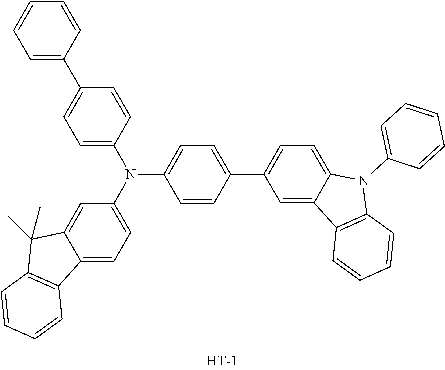 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | 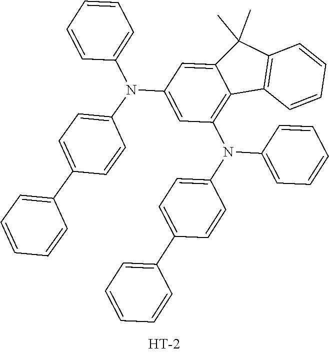 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| Light-Emitting Layer | 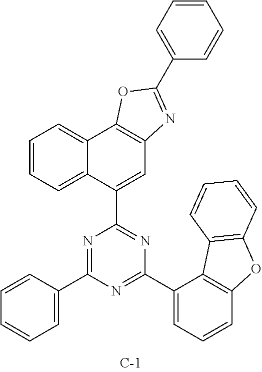 [SMILES: c1ccc(-c2nc(-c3cc4nc(-c5ccccc5)oc4c4ccccc34)nc(-c3cccc4oc5ccccc5c34)n2)cc1] |
| | 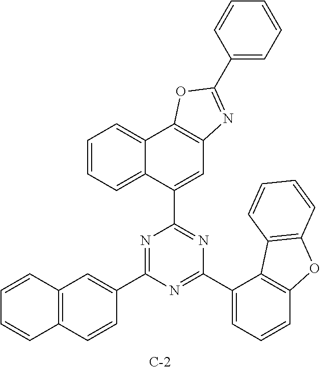 [SMILES: c1ccc(-c2nc3cc(-c4nc(-c5ccc6ccccc6c5)nc(-c5cccc6oc7ccccc7c56)n4)c4ccccc4c3o2)cc1] |
| | 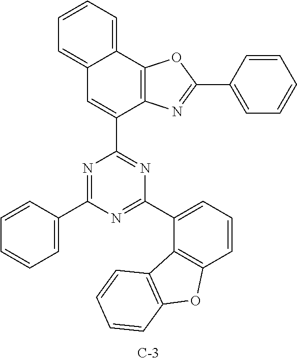 [SMILES: c1ccc(-c2nc(-c3cc4ccccc4c4oc(-c5ccccc5)nc34)nc(-c3cccc4oc5ccccc5c34)n2)cc1] |
| | 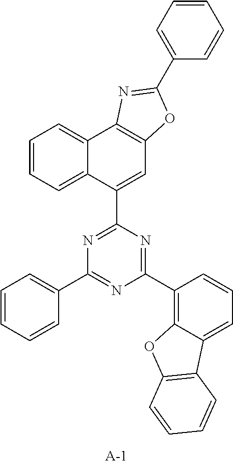 [SMILES: c1ccc(-c2nc(-c3cc4oc(-c5ccccc5)nc4c4ccccc34)nc(-c3cccc4c3oc3ccccc34)n2)cc1] |
| | 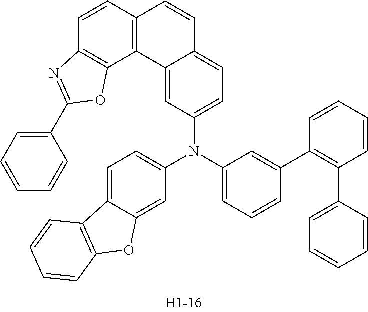 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | 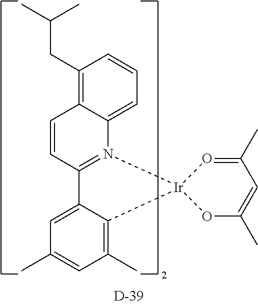 |
| Electron Transport Layer/Electron Injection Layer | 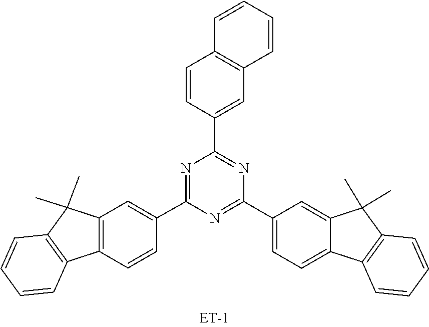 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 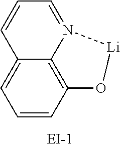 |

1. A plurality of host materials comprising at least one first host compound(s) and at least one second host compound(s), wherein the first host compound is represented by the following Formula 1, and the second host compound is represented by the following Formula 2. 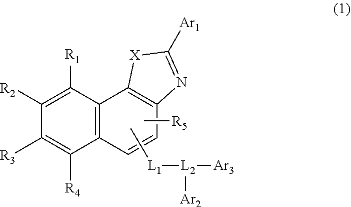
wherein,
X represents —O— or —S—;
R₁ to R₅ each independently represent, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to- 30-membered)heteroaryl, a substituted or unsubstituted (C1-C30)alkoxy, or —NRₐR_{b};
Rₐ and R_{b} each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to- 30-membered)heteroaryl;
L₁ represents a single bond or a substituted or unsubstituted (C6-C30)arylene;
L₂ represents a substituted or unsubstituted nitrogen-containing (3- to- 30-membered)heteroarylene; and
Ar₁ to Ar₃ each independently represent, a substituted or unsubstituted (C6-C30)aryl or a substituted or unsubstituted (3- to- 30-membered)heteroaryl; provided that Ar₂ and Ar₃ are different from each other; 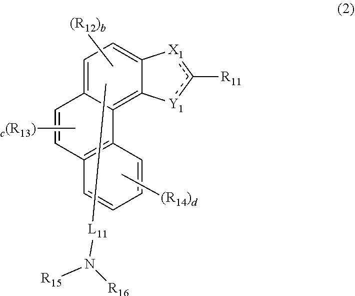
wherein,
X₁ and Y₁ each independently represent, —N═, —NR_{c}, —O— or —S—, provided that any one of X₁ and Y₁ is —N═ and the other of X₁ and Y₁ is —NR_{c}—, —O—, or —S—;
R₁₁ represents a substituted or unsubstituted (C6-C30)aryl or a substituted or unsubstituted (3- to- 30-membered)heteroaryl;
R₁₂ to R₁₄, and R_{c} each independently represent, hydrogen, deuterium, halogen, cyano, a substituted or unsubstituted (C1-C30)alkyl, a substituted or unsubstituted (C6-C30)aryl, a substituted or unsubstituted (3- to- 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -L₁₂-N(Ar₁₁)(Ar₁₂); or may be linked to the adjacent substituents to form a ring(s);
R₁₅ and R₁₆ each independently represent, a substituted or unsubstituted (C6-C30)aryl or a substituted or unsubstituted (3- to- 30-membered)heteroaryl;
L₁₁ and L₁₂ each independently represent, a single bond, a substituted or unsubstituted (C6-C30)arylene, or a substituted or unsubstituted (3- to- 30-membered)heteroarylene;
Ar₁₁ and Ar₁₂ each independently represent, hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (3- to- 30-membered)heteroaryl; and
b and c each independently represent, an integer of 1 or 2, and d represents an integer of 1 to 4, and when b to d are an integer of 2 or more, each of R₁₂ to R₁₄ may be the same or different.

2. The plurality of host materials according to claim 1, wherein L₂ in formula 1 represents a substituted or unsubstituted pyridylene, a substituted or unsubstituted pyrimidinylene, a substituted or unsubstituted triazinylene, a substituted or unsubstituted quinazolinylene, a substituted or unsubstituted quinoxalinylene, a substituted or unsubstituted benzoquinazolinylene, or a substituted or unsubstituted benzoquinoxalinylene.

3. The plurality of host materials according to claim 1, wherein at least one of Ar₂ and Ar₃ in formula 1 represents a substituted or unsubstituted dibenzofuranyl, a substituted or unsubstituted dibenzothiophenyl, or a substituted or unsubstituted dibenzoselenophenyl.

4. The plurality of host materials according to claim 1, wherein the substituted alkyl, the substituted alkenyl, the substituted cycloalkyl, the substituted aryl(ene), the substituted heteroaryl(ene), the substituted alkoxy, the substituted trialkylsilyl, the substituted dialkylarylsilyl, the substituted alkyldiarylsilyl, the substituted triarylsilyl, and the substituted fused ring of aliphatic ring and aromatic ring each independently are substituted with at least one selected from the group consisting of deuterium; halogen; cyano; carboxyl; nitro; hydroxyl; phosphine oxide; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (C6-C30)aryl unsubstituted or substituted with at least one of (C1-C30)alkyl and di(C6-C30)arylamino; (3- to- 30-membered)heteroaryl unsubstituted or substituted with at least one (C6-C30)aryl; tri(C1-C30)alkylsilyl; tri(C6-C30)arylsilyl; di(C1-C30)alkyl(C6-C30)arylsilyl; (C1-C30)alkyldi(C6-C30)arylsilyl; a fused ring of (C3-C30) aliphatic ring and (C6-C30) aromatic ring; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to- 30-membered)heteroarylamino; (C1-C30)alkyl(C2-C30)alkenylamino; (C1-C30)alkyl(C6-C30)arylamino; (C1-C30)alkyl(3- to- 30-membered)heteroarylamino; (C2-C30)alkenyl(C6-C30)arylamino; (C2-C30)alkenyl(3- to-30-membered)heteroarylamino; (C6-C30)aryl(3- to- 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)are(C1-C30)alkyl; and (C1-C30)alkyl(C6-C30)aryl.

5. The plurality of host materials according to claim 1, wherein the compound represented by the Formula 1 is selected from the following compounds:

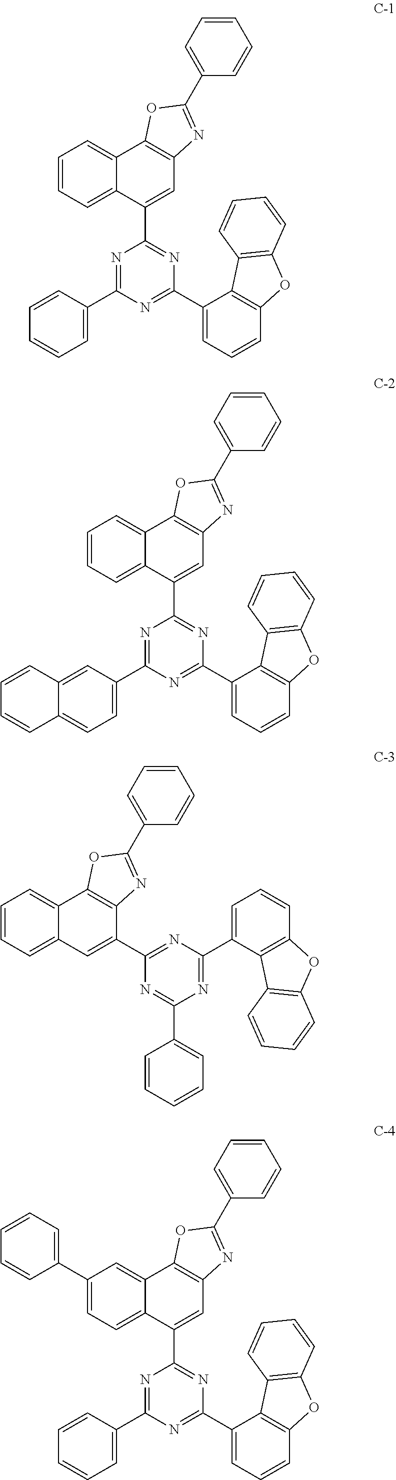

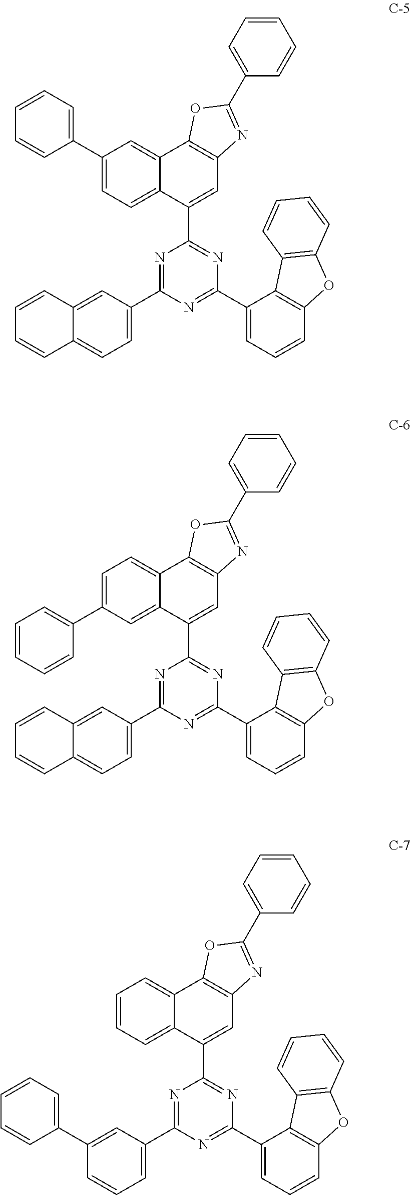 [SMILES: c1ccc(-c2ccc3c(-c4nc(-c5ccc6ccccc6c5)nc(-c5cccc6oc7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5oc6ccccc6c45)n2)cc2nc(-c4ccccc4)oc23)cc1 ; c1ccc(-c2cccc(-c3nc(-c4cc5nc(-c6ccccc6)oc5c5ccccc45)nc(-c4cccc5oc6ccccc6c45)n3)c2)cc1]

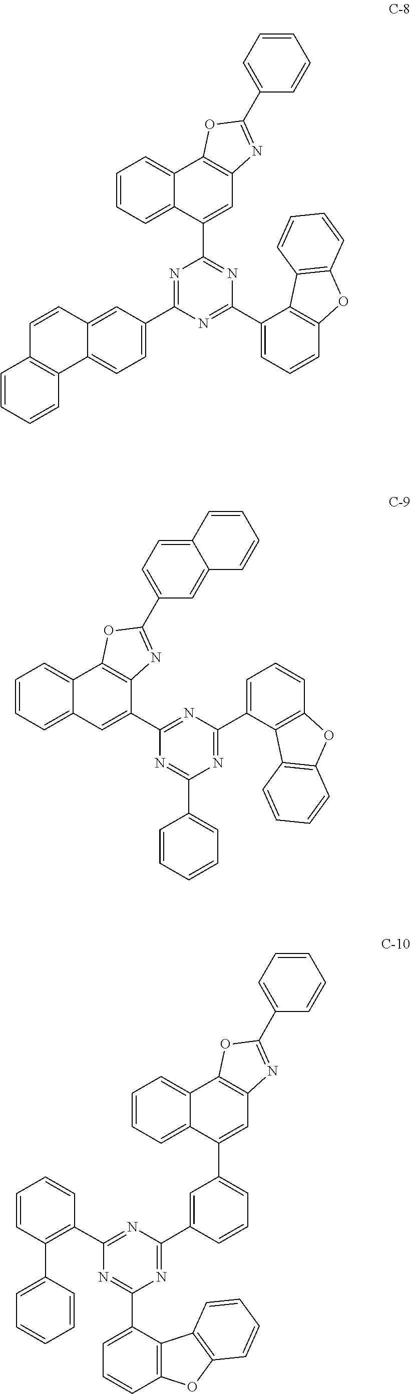 [SMILES: c1ccc(-c2nc3cc(-c4nc(-c5ccc6c(ccc7ccccc76)c5)nc(-c5cccc6oc7ccccc7c56)n4)c4ccccc4c3o2)cc1 ; c1ccc(-c2nc(-c3cc4ccccc4c4oc(-c5ccc6ccccc6c5)nc34)nc(-c3cccc4oc5ccccc5c34)n2)cc1 ; c1ccc(-c2nc3cc(-c4cccc(-c5nc(-c6ccccc6-c6ccccc6)nc(-c6cccc7oc8ccccc8c67)n5)c4)c4ccccc4c3o2)cc1]

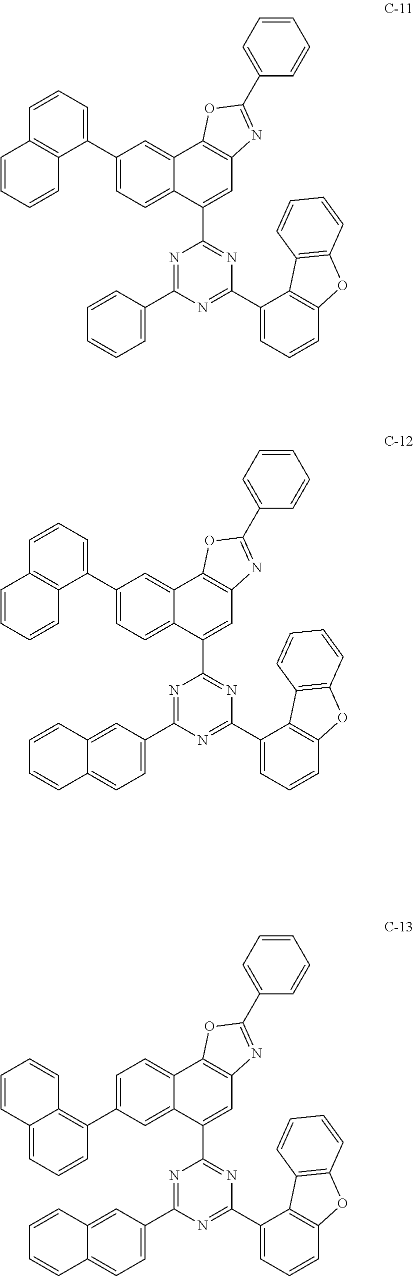 [SMILES: c1ccc(-c2nc(-c3cc4nc(-c5ccccc5)oc4c4cc(-c5cccc6ccccc56)ccc34)nc(-c3cccc4oc5ccccc5c34)n2)cc1 ; c1ccc(-c2nc3cc(-c4nc(-c5ccc6ccccc6c5)nc(-c5cccc6oc7ccccc7c56)n4)c4ccc(-c5cccc6ccccc56)cc4c3o2)cc1 ; c1ccc(-c2nc3cc(-c4nc(-c5ccc6ccccc6c5)nc(-c5cccc6oc7ccccc7c56)n4)c4cc(-c5cccc6ccccc56)ccc4c3o2)cc1]

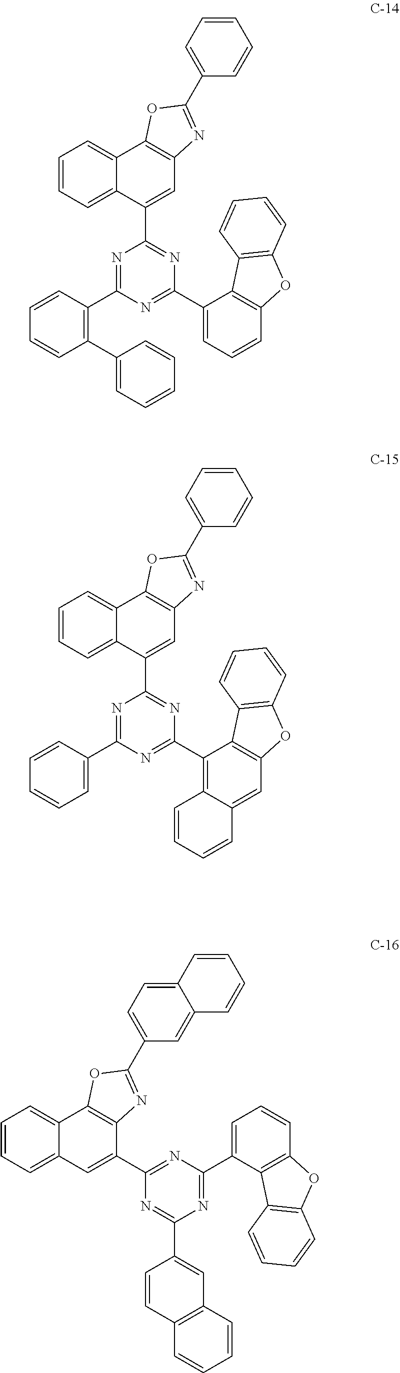 [SMILES: c1ccc(-c2nc3cc(-c4nc(-c5ccccc5-c5ccccc5)nc(-c5cccc6oc7ccccc7c56)n4)c4ccccc4c3o2)cc1 ; c1ccc(-c2nc(-c3cc4nc(-c5ccccc5)oc4c4ccccc34)nc(-c3c4ccccc4cc4oc5ccccc5c34)n2)cc1 ; c1ccc2cc(-c3nc(-c4cc5ccccc5c5oc(-c6ccc7ccccc7c6)nc45)nc(-c4cccc5oc6ccccc6c45)n3)ccc2c1]

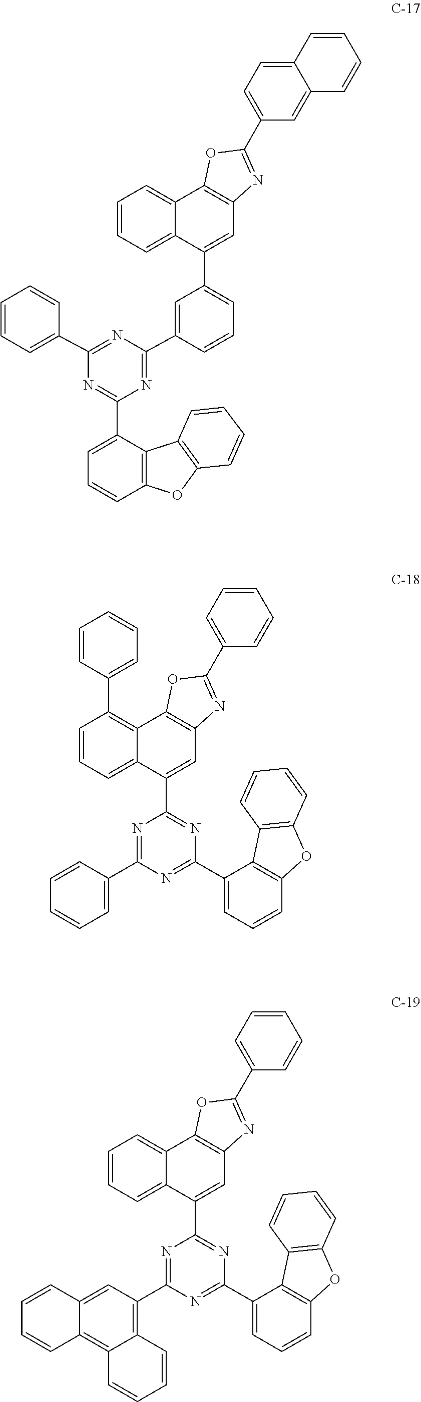 [SMILES: c1ccc(-c2nc(-c3cccc(-c4cc5nc(-c6ccc7ccccc7c6)oc5c5ccccc45)c3)nc(-c3cccc4oc5ccccc5c34)n2)cc1 ; c1ccc(-c2nc(-c3cc4nc(-c5ccccc5)oc4c4c(-c5ccccc5)cccc34)nc(-c3cccc4oc5ccccc5c34)n2)cc1 ; c1ccc(-c2nc3cc(-c4nc(-c5cc6ccccc6c6ccccc56)nc(-c5cccc6oc7ccccc7c56)n4)c4ccccc4c3o2)cc1]

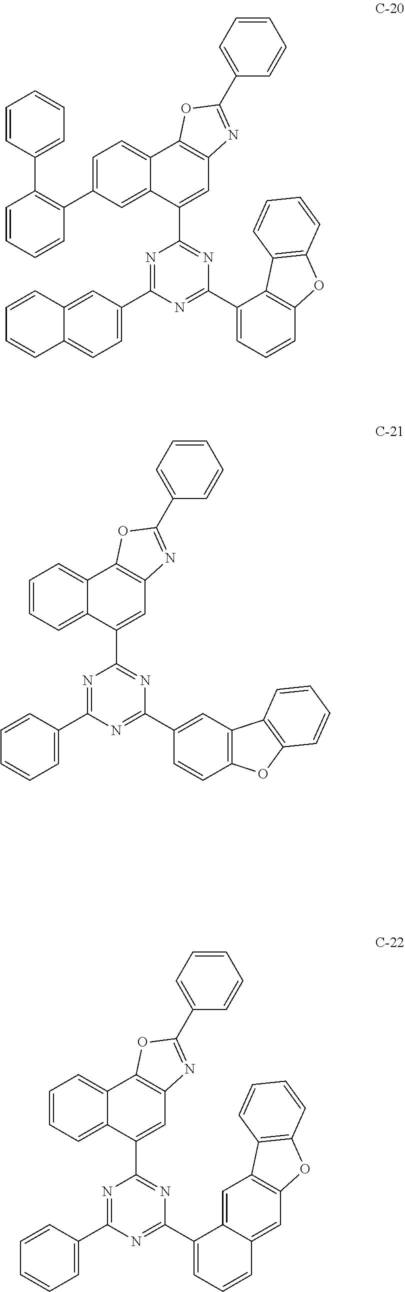 [SMILES: c1ccc(-c2nc3cc(-c4nc(-c5ccc6ccccc6c5)nc(-c5cccc6oc7ccccc7c56)n4)c4cc(-c5ccccc5-c5ccccc5)ccc4c3o2)cc1 ; c1ccc(-c2nc(-c3ccc4oc5ccccc5c4c3)nc(-c3cc4nc(-c5ccccc5)oc4c4ccccc34)n2)cc1 ; c1ccc(-c2nc(-c3cccc4cc5oc6ccccc6c5cc34)nc(-c3cc4nc(-c5ccccc5)oc4c4ccccc34)n2)cc1]

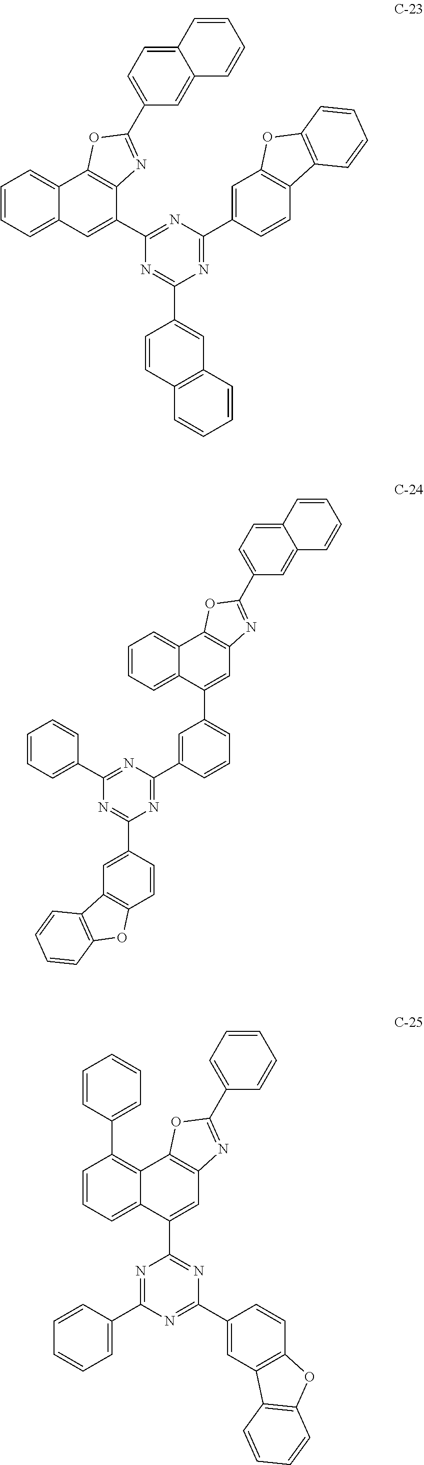 [SMILES: c1ccc2cc(-c3nc(-c4ccc5c(c4)oc4ccccc45)nc(-c4cc5ccccc5c5oc(-c6ccc7ccccc7c6)nc45)n3)ccc2c1 ; c1ccc(-c2nc(-c3cccc(-c4cc5nc(-c6ccc7ccccc7c6)oc5c5ccccc45)c3)nc(-c3ccc4oc5ccccc5c4c3)n2)cc1 ; c1ccc(-c2nc(-c3ccc4oc5ccccc5c4c3)nc(-c3cc4nc(-c5ccccc5)oc4c4c(-c5ccccc5)cccc34)n2)cc1]

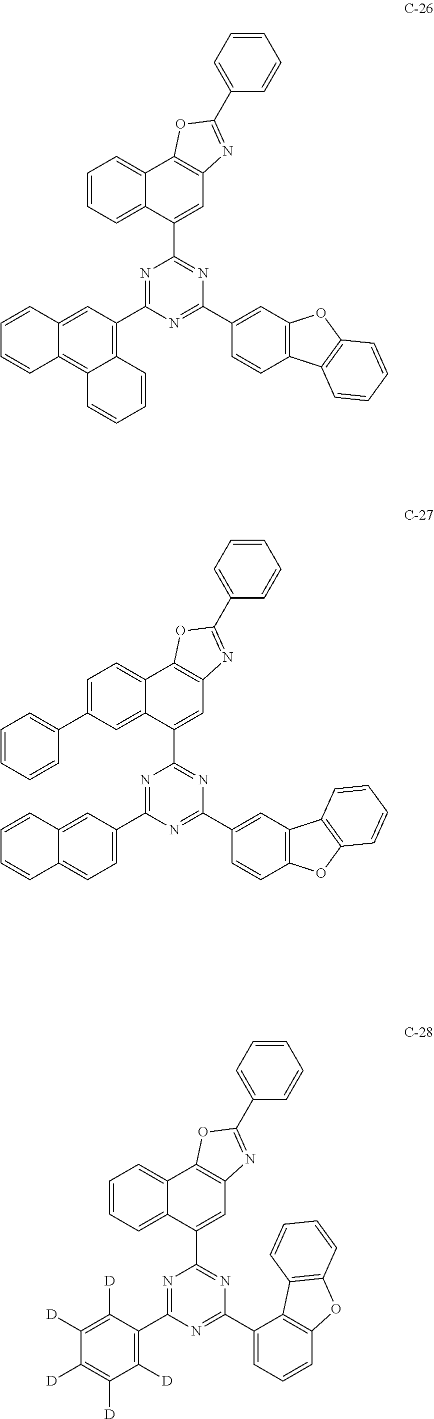 [SMILES: c1ccc(-c2nc3cc(-c4nc(-c5ccc6c(c5)oc5ccccc56)nc(-c5cc6ccccc6c6ccccc56)n4)c4ccccc4c3o2)cc1 ; c1ccc(-c2ccc3c(c2)c(-c2nc(-c4ccc5ccccc5c4)nc(-c4ccc5oc6ccccc6c5c4)n2)cc2nc(-c4ccccc4)oc23)cc1 ; [2H]c1c([2H])c([2H])c(-c2nc(-c3cc4nc(-c5ccccc5)oc4c4ccccc34)nc(-c3cccc4oc5ccccc5c34)n2)c([2H])c1[2H]]

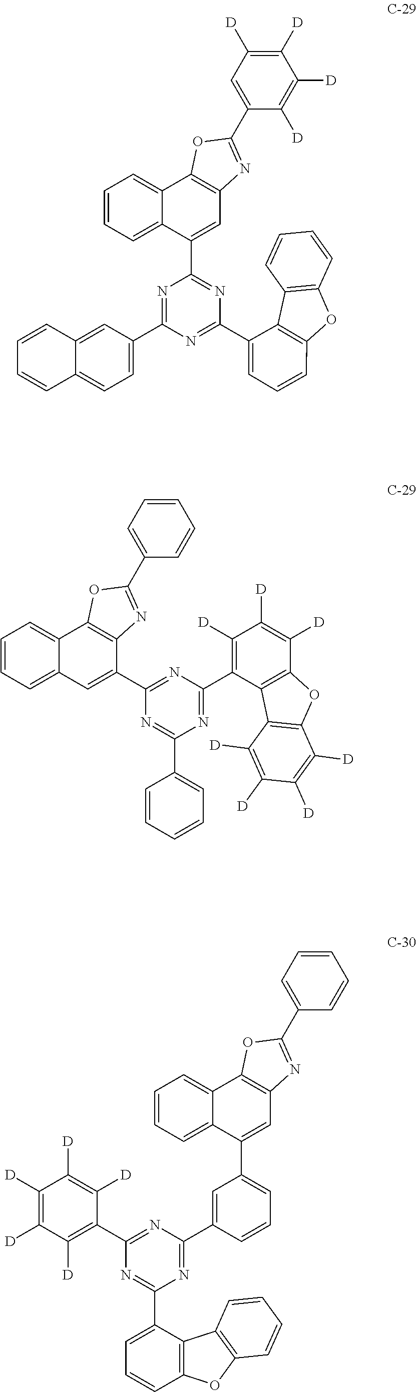 [SMILES: [2H]c1cc(-c2nc3cc(-c4nc(-c5ccc6ccccc6c5)nc(-c5cccc6oc7ccccc7c56)n4)c4ccccc4c3o2)c([2H])c([2H])c1[2H] ; [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H] ; [2H]c1c([2H])c([2H])c(-c2nc(-c3cccc(-c4cc5nc(-c6ccccc6)oc5c5ccccc45)c3)nc(-c3cccc4oc5ccccc5c34)n2)c([2H])c1[2H]]

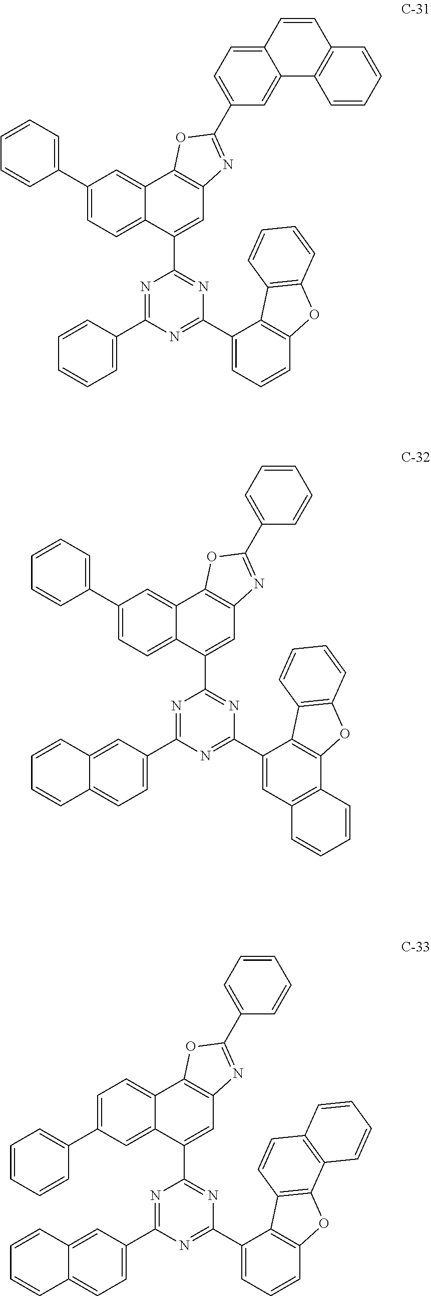 [SMILES: c1ccc(-c2ccc3c(-c4nc(-c5ccccc5)nc(-c5cccc6oc7ccccc7c56)n4)cc4nc(-c5ccc6ccc7ccccc7c6c5)oc4c3c2)cc1 ; c1ccc(-c2ccc3c(-c4nc(-c5ccc6ccccc6c5)nc(-c5cc6ccccc6c6oc7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5oc6c7ccccc7ccc6c45)n2)cc2nc(-c4ccccc4)oc23)cc1]

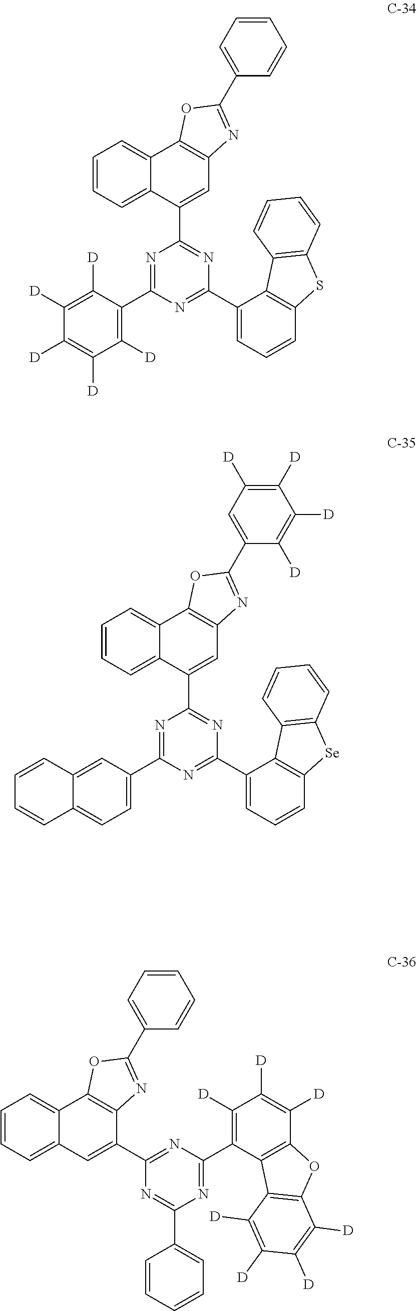 [SMILES: [2H]c1c([2H])c([2H])c(-c2nc(-c3cc4nc(-c5ccccc5)oc4c4ccccc34)nc(-c3cccc4sc5ccccc5c34)n2)c([2H])c1[2H] ; [2H]c1cc(-c2nc3cc(-c4nc(-c5ccc6ccccc6c5)nc(-c5cccc6[se]c7ccccc7c56)n4)c4ccccc4c3o2)c([2H])c([2H])c1[2H] ; [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H]]

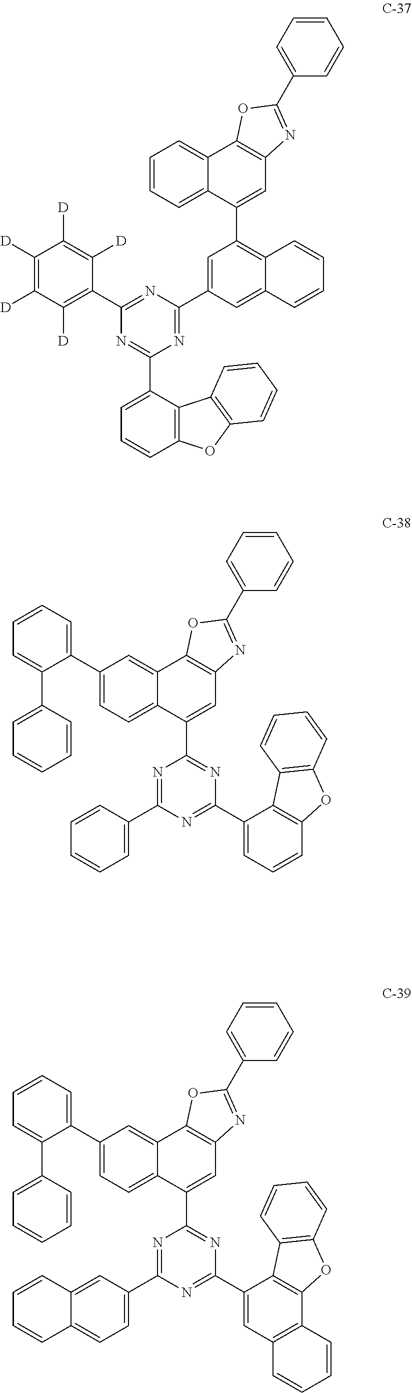 [SMILES: [2H]c1c([2H])c([2H])c(-c2nc(-c3cc(-c4cc5nc(-c6ccccc6)oc5c5ccccc45)c4ccccc4c3)nc(-c3cccc4oc5ccccc5c34)n2)c([2H])c1[2H] ; c1ccc(-c2nc(-c3cc4nc(-c5ccccc5)oc4c4cc(-c5ccccc5-c5ccccc5)ccc34)nc(-c3cccc4oc5ccccc5c34)n2)cc1 ; c1ccc(-c2nc3cc(-c4nc(-c5ccc6ccccc6c5)nc(-c5cc6ccccc6c6oc7ccccc7c56)n4)c4ccc(-c5ccccc5-c5ccccc5)cc4c3o2)cc1]

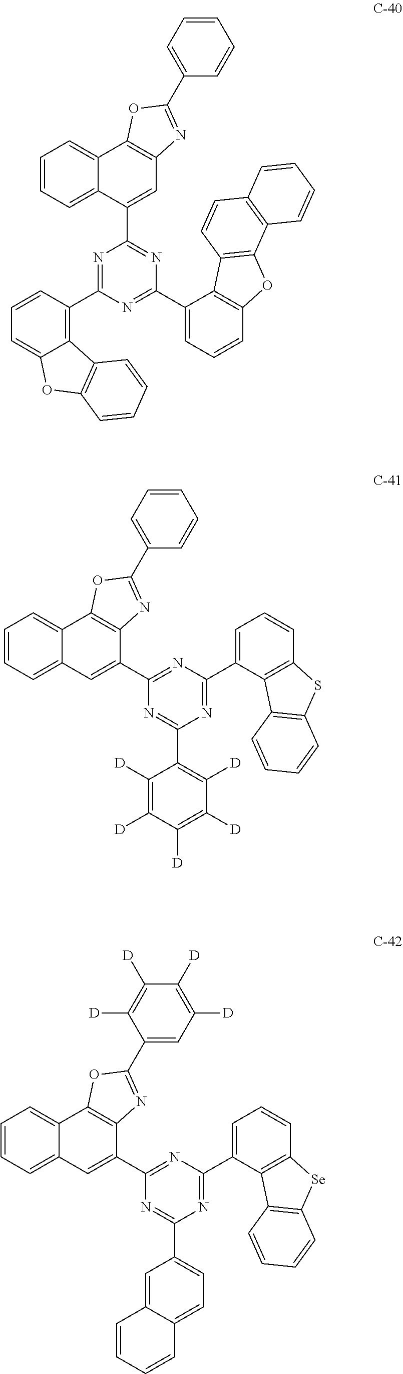 [SMILES: c1ccc(-c2nc3cc(-c4nc(-c5cccc6oc7ccccc7c56)nc(-c5cccc6oc7c8ccccc8ccc7c56)n4)c4ccccc4c3o2)cc1 ; [2H]c1c([2H])c([2H])c(-c2nc(-c3cc4ccccc4c4oc(-c5ccccc5)nc34)nc(-c3cccc4sc5ccccc5c34)n2)c([2H])c1[2H] ; [2H]c1cc(-c2nc3c(-c4nc(-c5ccc6ccccc6c5)nc(-c5cccc6[se]c7ccccc7c56)n4)cc4ccccc4c3o2)c([2H])c([2H])c1[2H]]

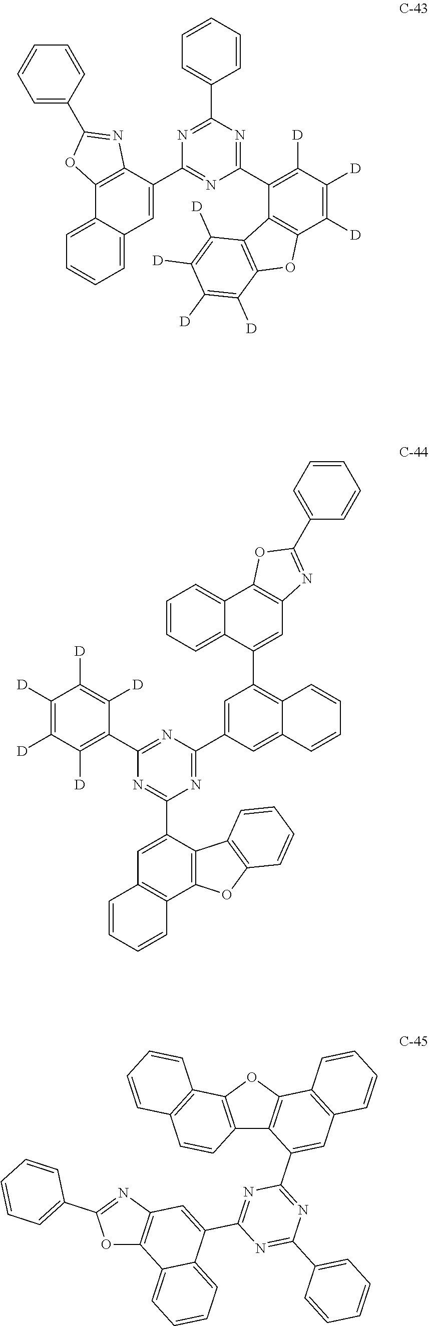 [SMILES: [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H] ; [2H]c1c([2H])c([2H])c(-c2nc(-c3cc(-c4cc5nc(-c6ccccc6)oc5c5ccccc45)c4ccccc4c3)nc(-c3cc4ccccc4c4oc5ccccc5c34)n2)c([2H])c1[2H] ; c1ccc(-c2nc(-c3cc4nc(-c5ccccc5)oc4c4ccccc34)nc(-c3cc4ccccc4c4oc5c6ccccc6ccc5c34)n2)cc1]

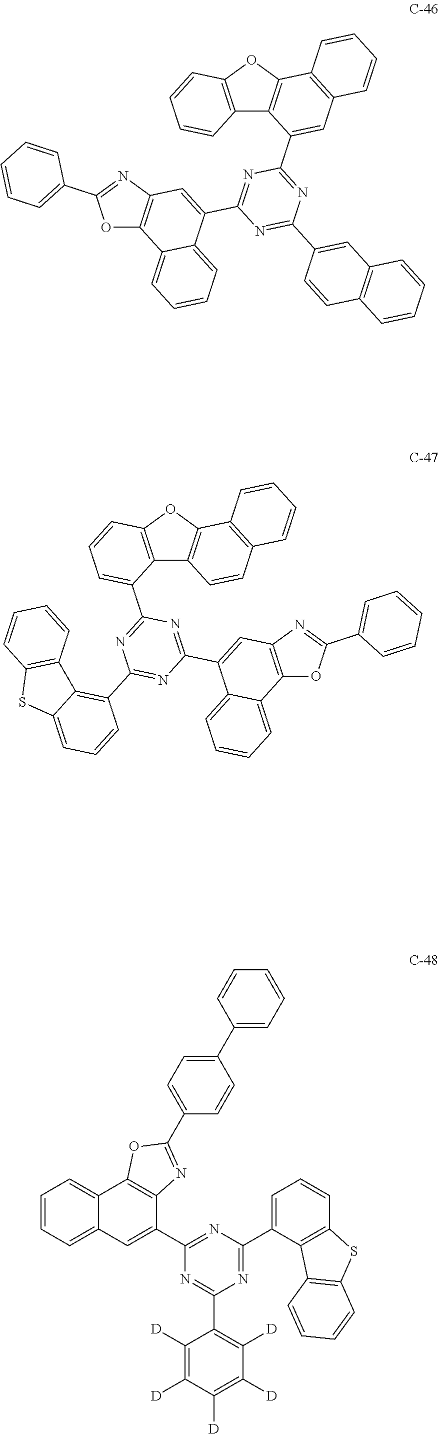 [SMILES: c1ccc(-c2nc3cc(-c4nc(-c5ccc6ccccc6c5)nc(-c5cc6ccccc6c6oc7ccccc7c56)n4)c4ccccc4c3o2)cc1 ; c1ccc(-c2nc3cc(-c4nc(-c5cccc6oc7c8ccccc8ccc7c56)nc(-c5cccc6sc7ccccc7c56)n4)c4ccccc4c3o2)cc1 ; [2H]c1c([2H])c([2H])c(-c2nc(-c3cc4ccccc4c4oc(-c5ccc(-c6ccccc6)cc5)nc34)nc(-c3cccc4sc5ccccc5c34)n2)c([2H])c1[2H]]

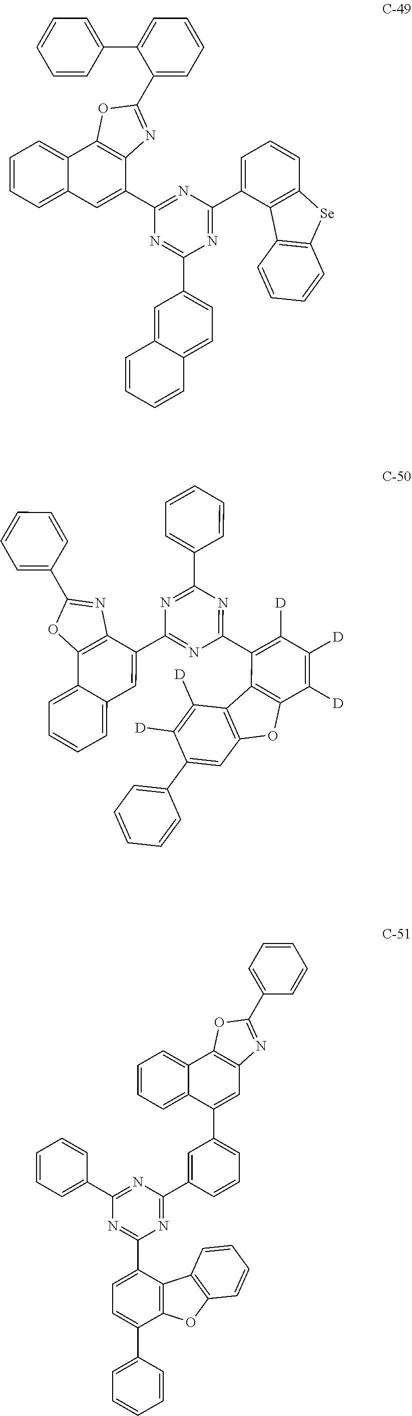 [SMILES: c1ccc(-c2ccccc2-c2nc3c(-c4nc(-c5ccc6ccccc6c5)nc(-c5cccc6[se]c7ccccc7c56)n4)cc4ccccc4c3o2)cc1 ; [2H]c1c([2H])c(-c2nc(-c3ccccc3)nc(-c3cc4ccccc4c4oc(-c5ccccc5)nc34)n2)c2c(oc3cc(-c4ccccc4)c([2H])c([2H])c32)c1[2H] ; c1ccc(-c2nc(-c3cccc(-c4cc5nc(-c6ccccc6)oc5c5ccccc45)c3)nc(-c3ccc(-c4ccccc4)c4oc5ccccc5c34)n2)cc1]

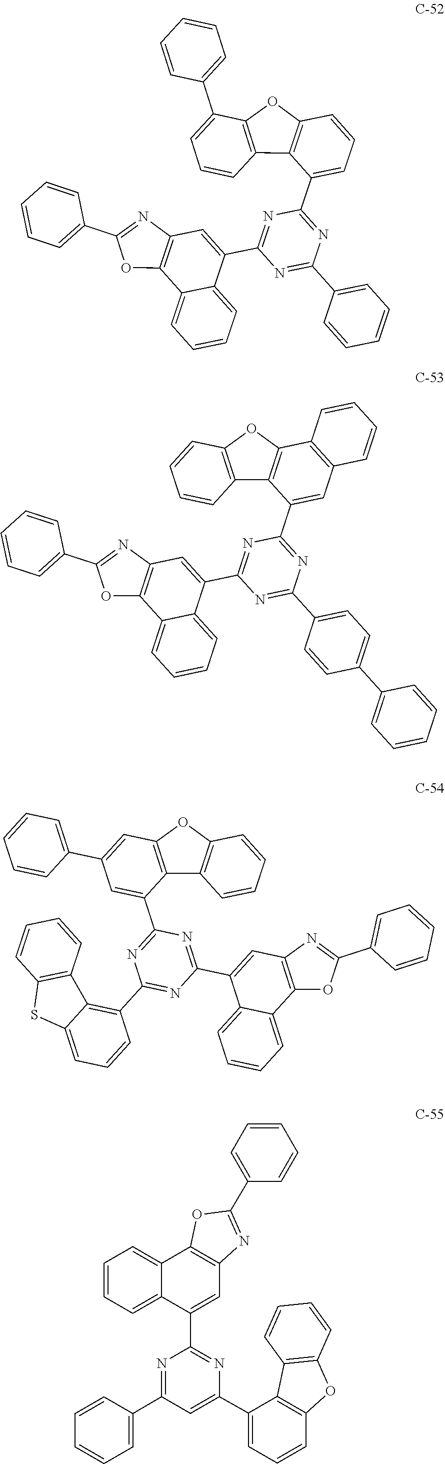 [SMILES: c1ccc(-c2nc(-c3cc4nc(-c5ccccc5)oc4c4ccccc34)nc(-c3cccc4oc5c(-c6ccccc6)cccc5c34)n2)cc1 ; c1ccc(-c2ccc(-c3nc(-c4cc5nc(-c6ccccc6)oc5c5ccccc45)nc(-c4cc5ccccc5c5oc6ccccc6c45)n3)cc2)cc1 ; c1ccc(-c2cc(-c3nc(-c4cc5nc(-c6ccccc6)oc5c5ccccc45)nc(-c4cccc5sc6ccccc6c45)n3)c3c(c2)oc2ccccc23)cc1 ; c1ccc(-c2cc(-c3cccc4oc5ccccc5c34)nc(-c3cc4nc(-c5ccccc5)oc4c4ccccc34)n2)cc1]

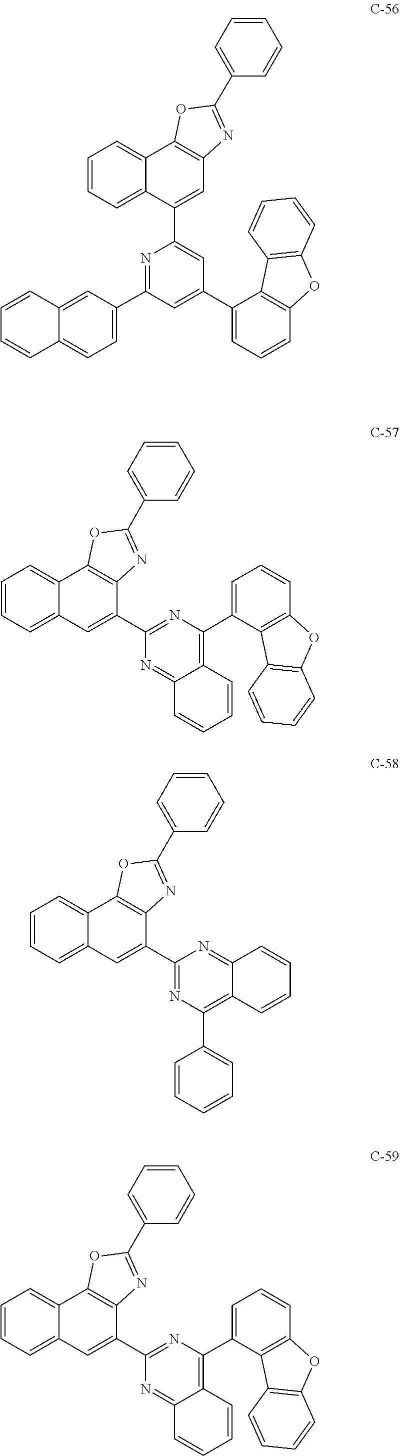

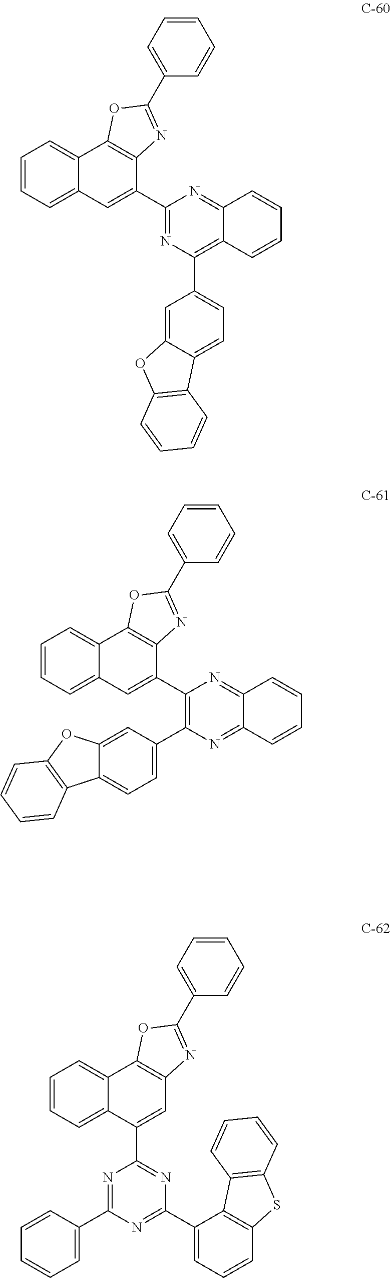 [SMILES: c1ccc(-c2nc3c(-c4nc(-c5ccc6c(c5)oc5ccccc56)c5ccccc5n4)cc4ccccc4c3o2)cc1 ; c1ccc(-c2nc3c(-c4nc5ccccc5nc4-c4ccc5c(c4)oc4ccccc45)cc4ccccc4c3o2)cc1 ; c1ccc(-c2nc(-c3cc4nc(-c5ccccc5)oc4c4ccccc34)nc(-c3cccc4sc5ccccc5c34)n2)cc1]

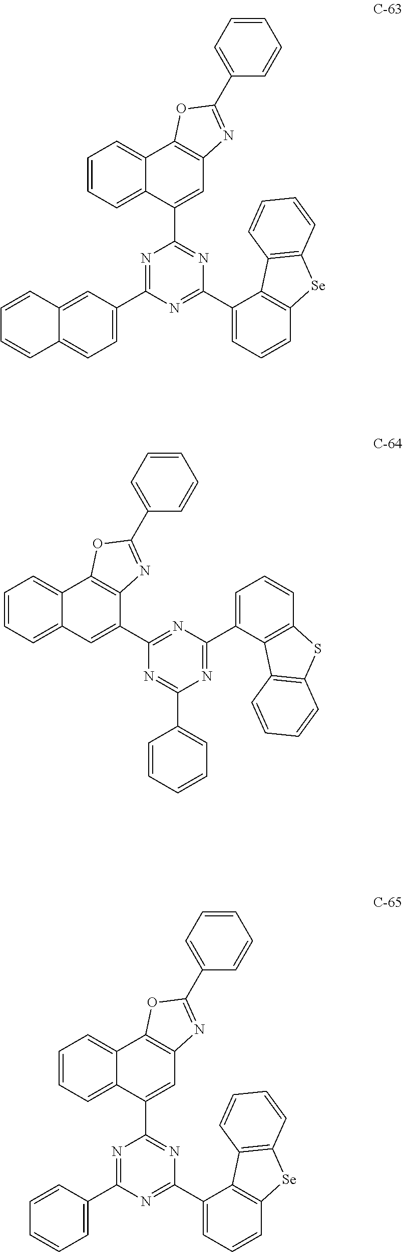 [SMILES: c1ccc(-c2nc3cc(-c4nc(-c5ccc6ccccc6c5)nc(-c5cccc6[se]c7ccccc7c56)n4)c4ccccc4c3o2)cc1 ; c1ccc(-c2nc(-c3cc4ccccc4c4oc(-c5ccccc5)nc34)nc(-c3cccc4sc5ccccc5c34)n2)cc1 ; c1ccc(-c2nc(-c3cc4nc(-c5ccccc5)oc4c4ccccc34)nc(-c3cccc4[se]c5ccccc5c34)n2)cc1]

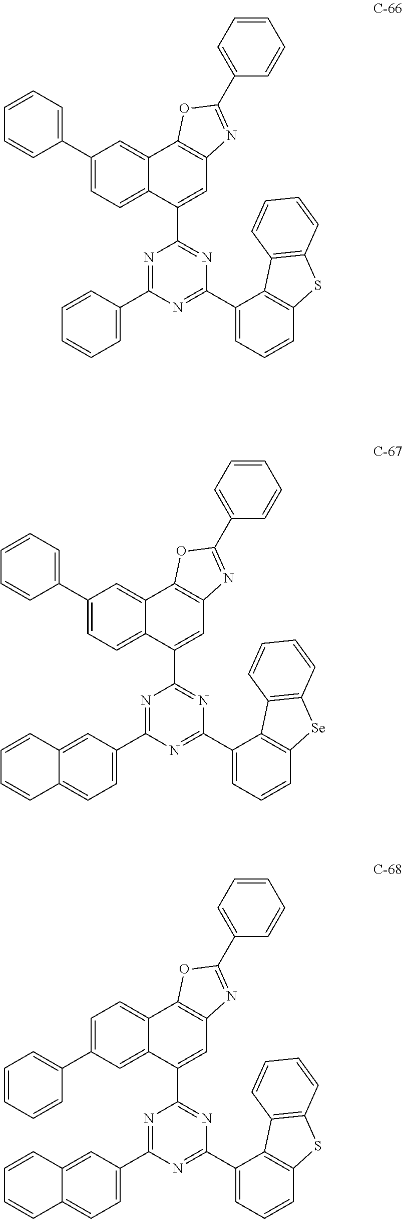 [SMILES: c1ccc(-c2ccc3c(-c4nc(-c5ccccc5)nc(-c5cccc6sc7ccccc7c56)n4)cc4nc(-c5ccccc5)oc4c3c2)cc1 ; c1ccc(-c2ccc3c(-c4nc(-c5ccc6ccccc6c5)nc(-c5cccc6[se]c7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5sc6ccccc6c45)n2)cc2nc(-c4ccccc4)oc23)cc1]

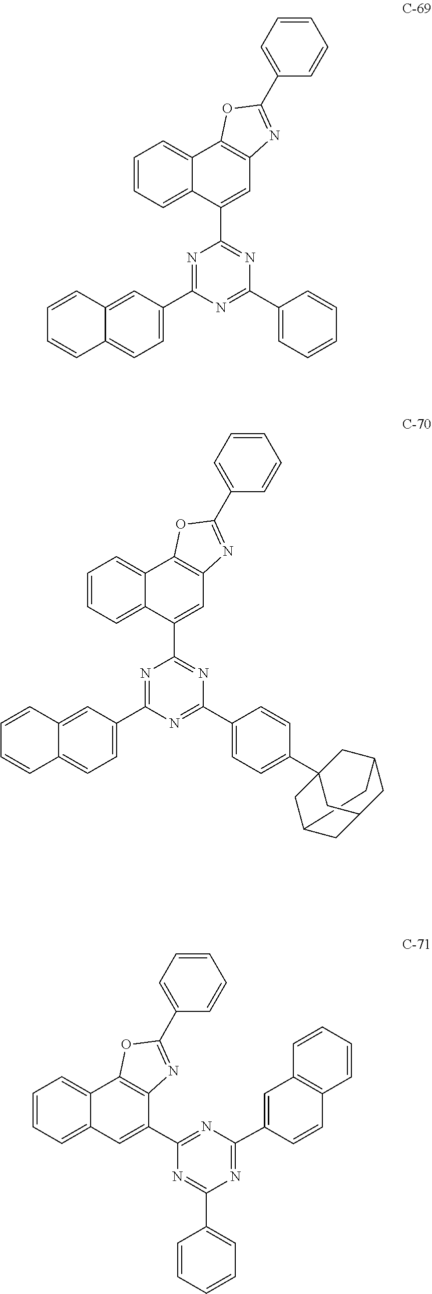 [SMILES: c1ccc(-c2nc(-c3ccc4ccccc4c3)nc(-c3cc4nc(-c5ccccc5)oc4c4ccccc34)n2)cc1 ; c1ccc(-c2nc3cc(-c4nc(-c5ccc(C67CC8CC(CC(C8)C6)C7)cc5)nc(-c5ccc6ccccc6c5)n4)c4ccccc4c3o2)cc1 ; c1ccc(-c2nc(-c3ccc4ccccc4c3)nc(-c3cc4ccccc4c4oc(-c5ccccc5)nc34)n2)cc1]

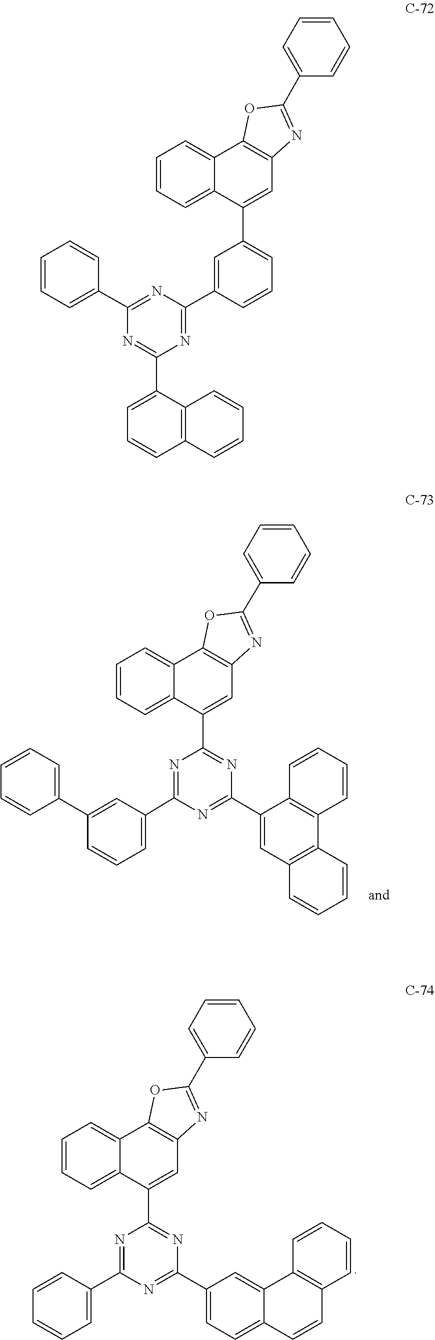 [SMILES: c1ccc(-c2nc(-c3cccc(-c4cc5nc(-c6ccccc6)oc5c5ccccc45)c3)nc(-c3cccc4ccccc34)n2)cc1 ; c1ccc(-c2cccc(-c3nc(-c4cc5ccccc5c5ccccc45)nc(-c4cc5nc(-c6ccccc6)oc5c5ccccc45)n3)c2)cc1 ; c1ccc(-c2nc(-c3ccc4ccc5ccccc5c4c3)nc(-c3cc4nc(-c5ccccc5)oc4c4ccccc34)n2)cc1]

6. The plurality of host materials according to claim 1, wherein the compound represented by the Formula 2 is selected from the following compounds:

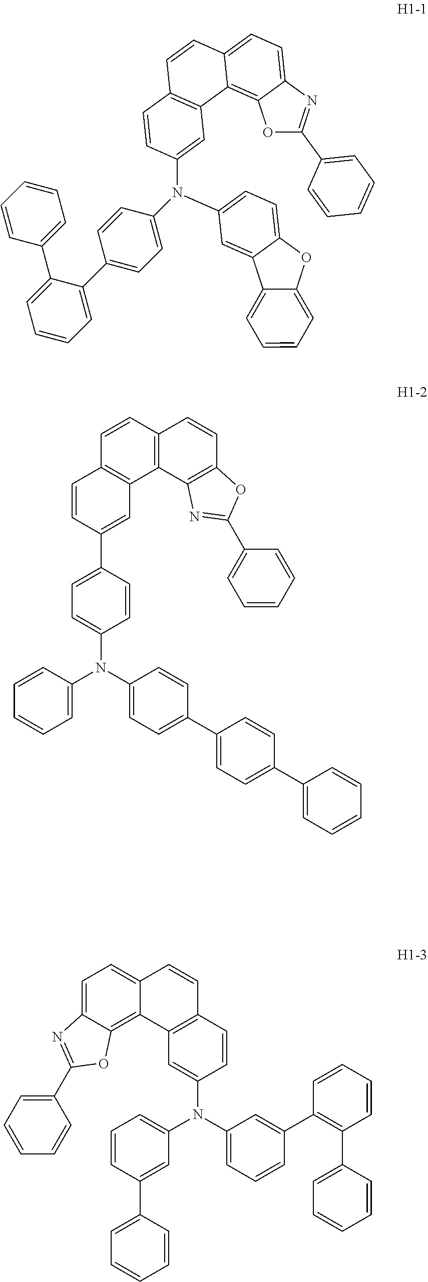 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

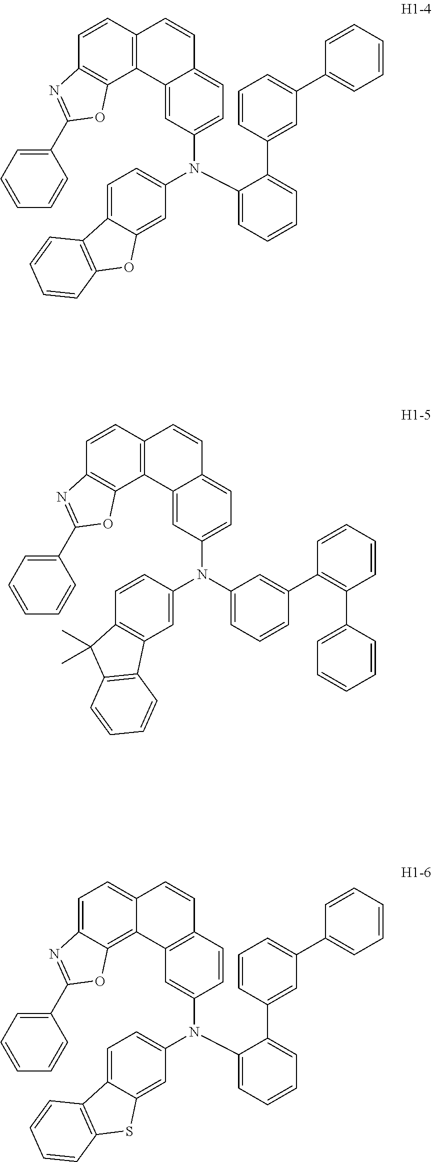 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

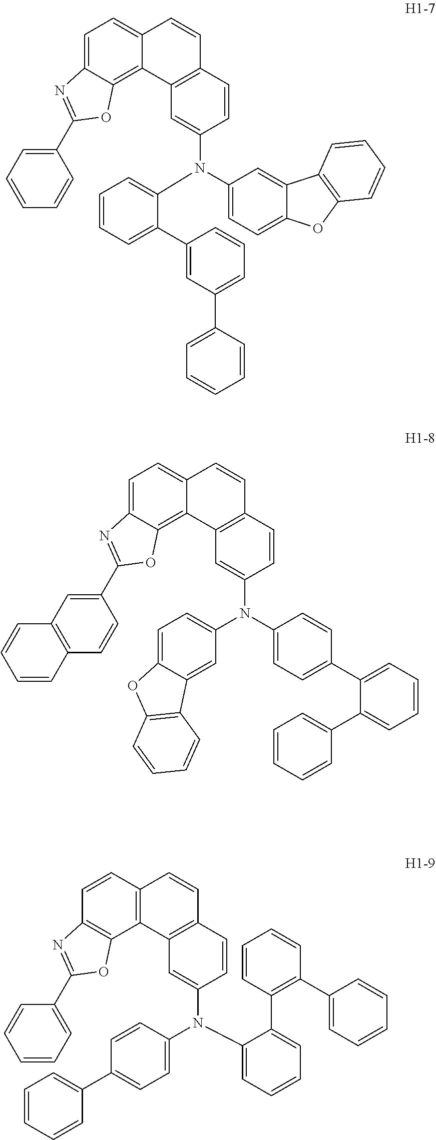 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1]

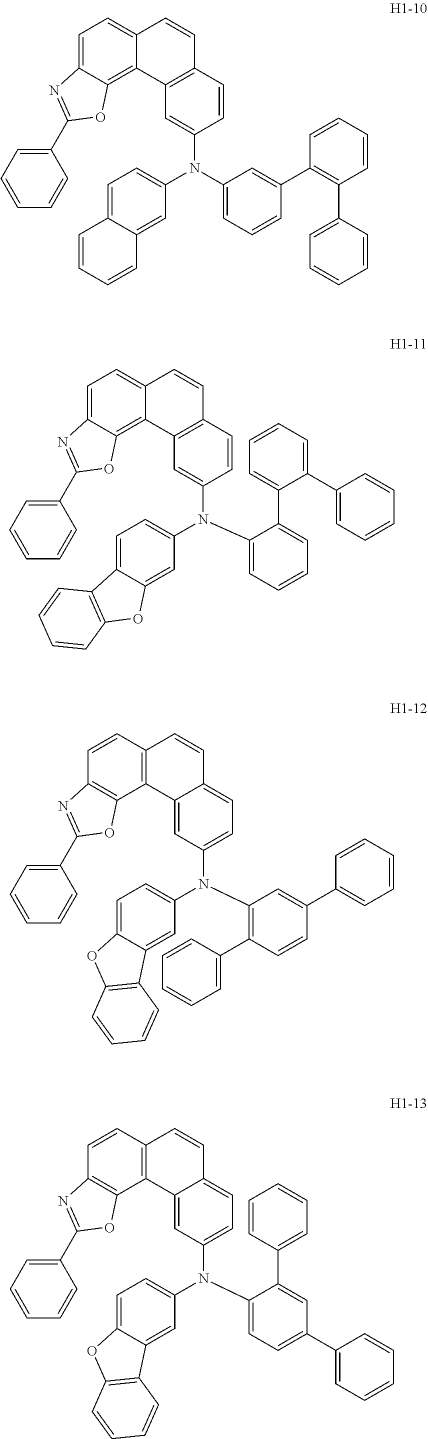 [SMILES: c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1]

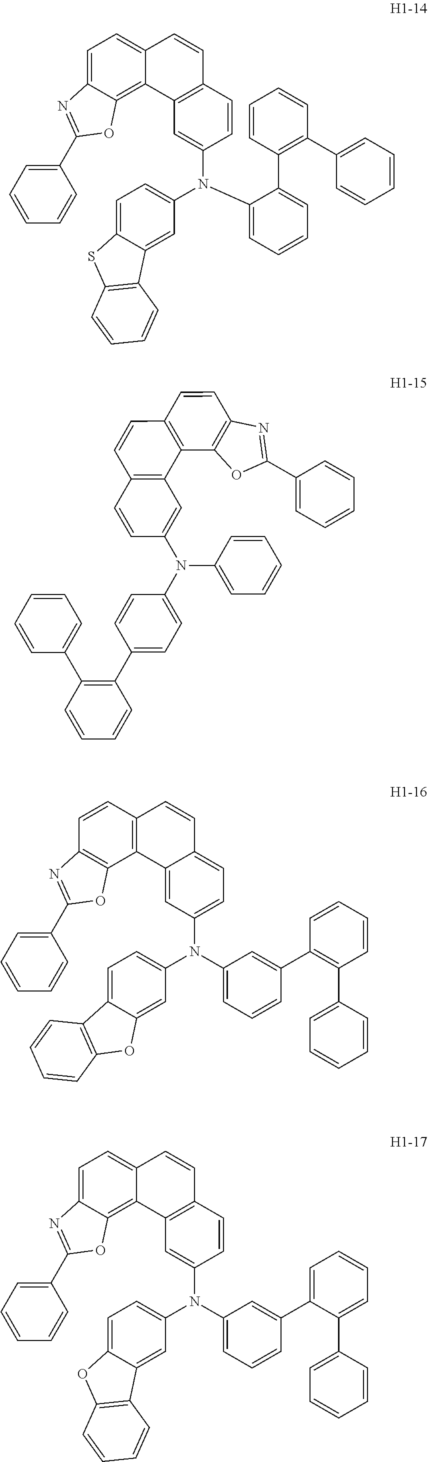 [SMILES: c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

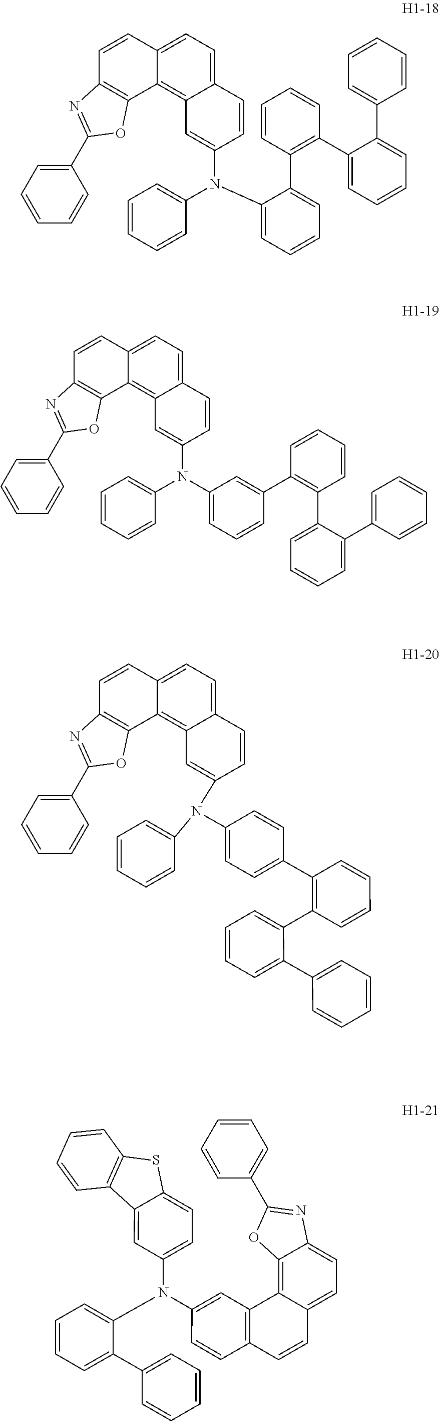 [SMILES: c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1 ; c1ccc(-c2nc3ccc4ccc5ccc(N(c6ccc7sc8ccccc8c7c6)c6ccccc6-c6ccccc6)cc5c4c3o2)cc1]

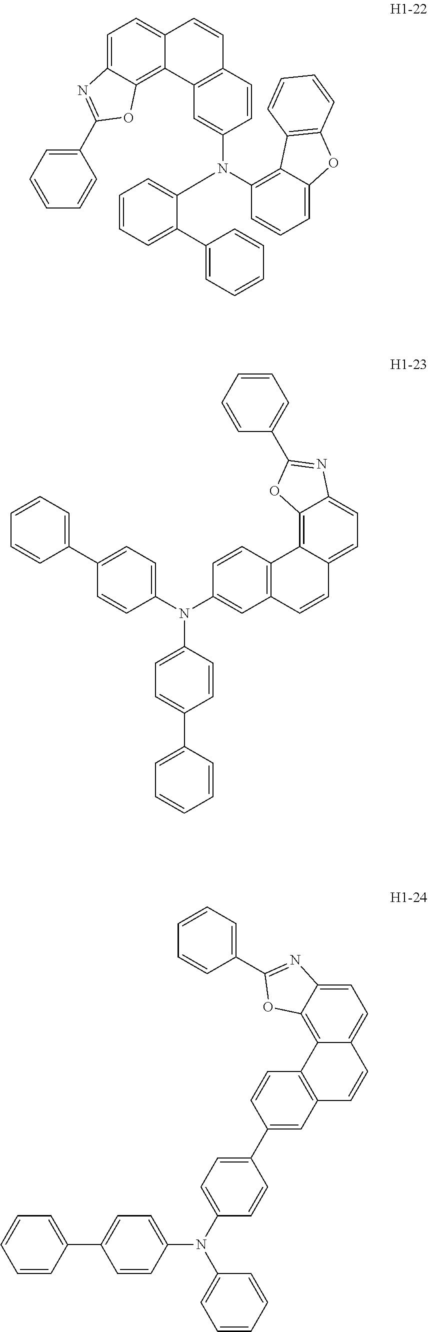 [SMILES: c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; c1ccc(-c2ccc(N(c3ccccc3)c3ccc(-c4ccc5c(ccc6ccc7nc(-c8ccccc8)oc7c65)c4)cc3)cc2)cc1]

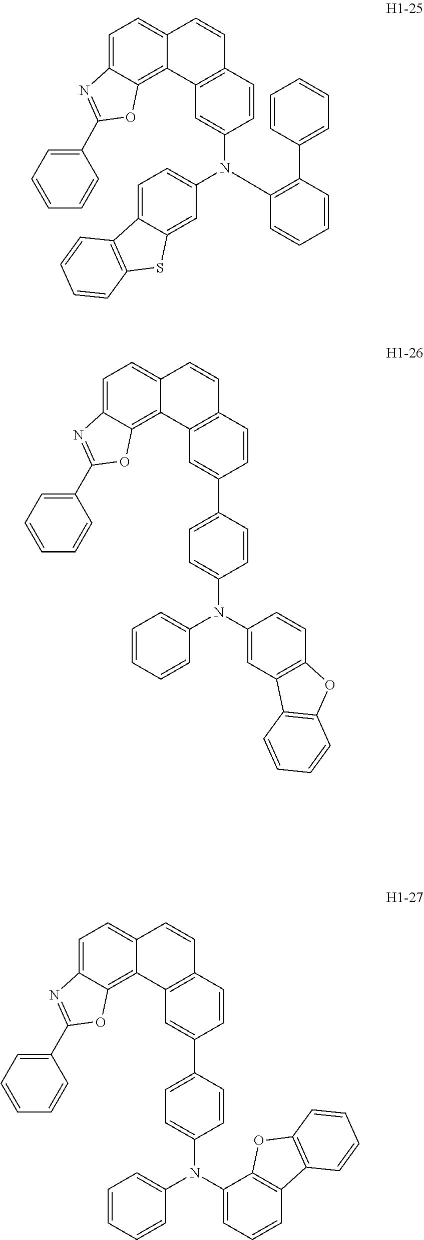 [SMILES: c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1 ; c1ccc(-c2nc3ccc4ccc5ccc(-c6ccc(N(c7ccccc7)c7cccc8c7oc7ccccc78)cc6)cc5c4c3o2)cc1]

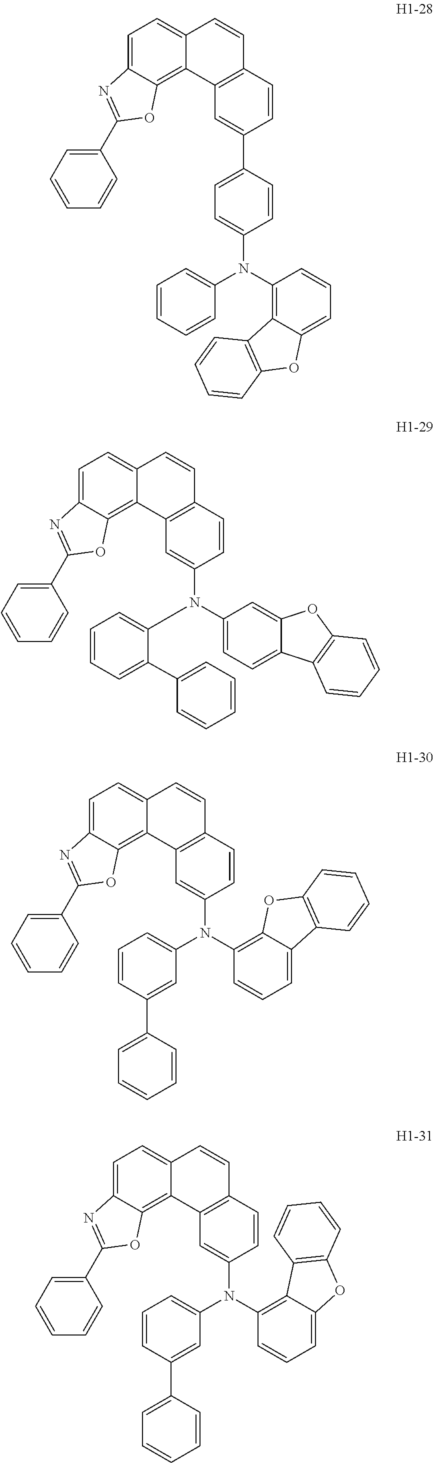

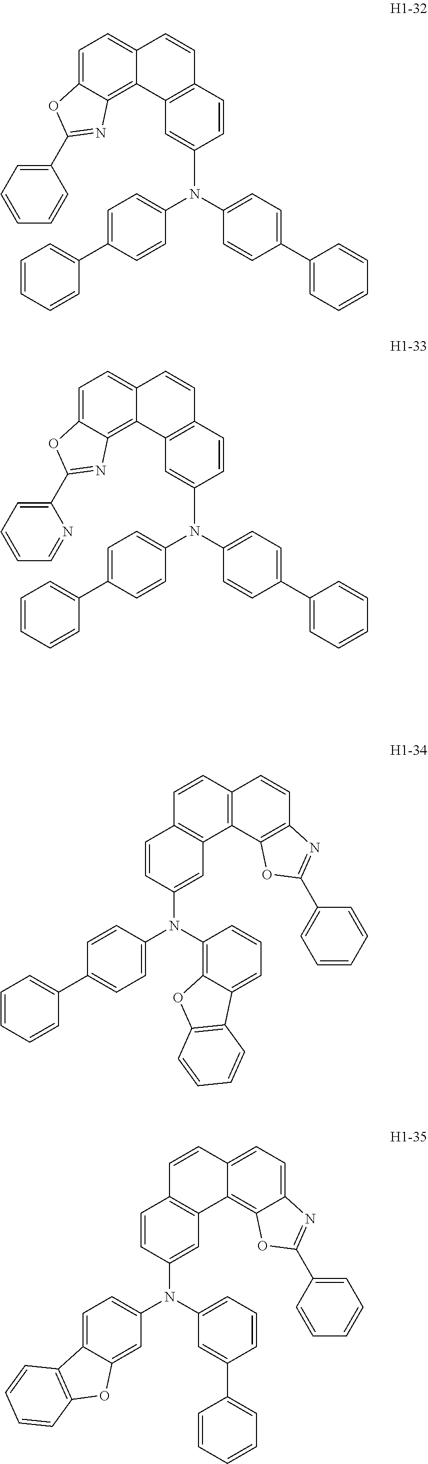 [SMILES: c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1]

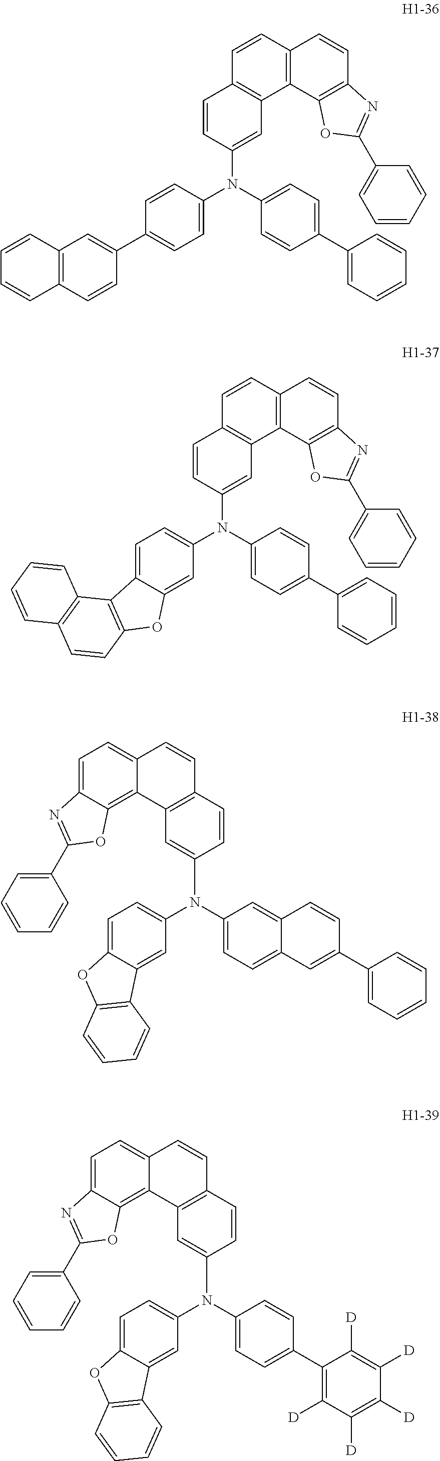 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H]]

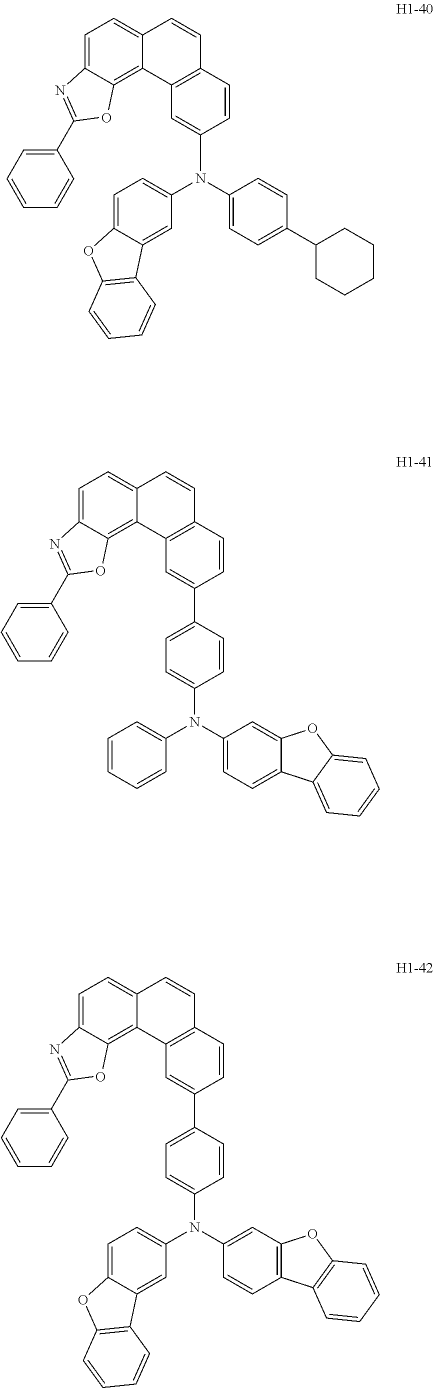

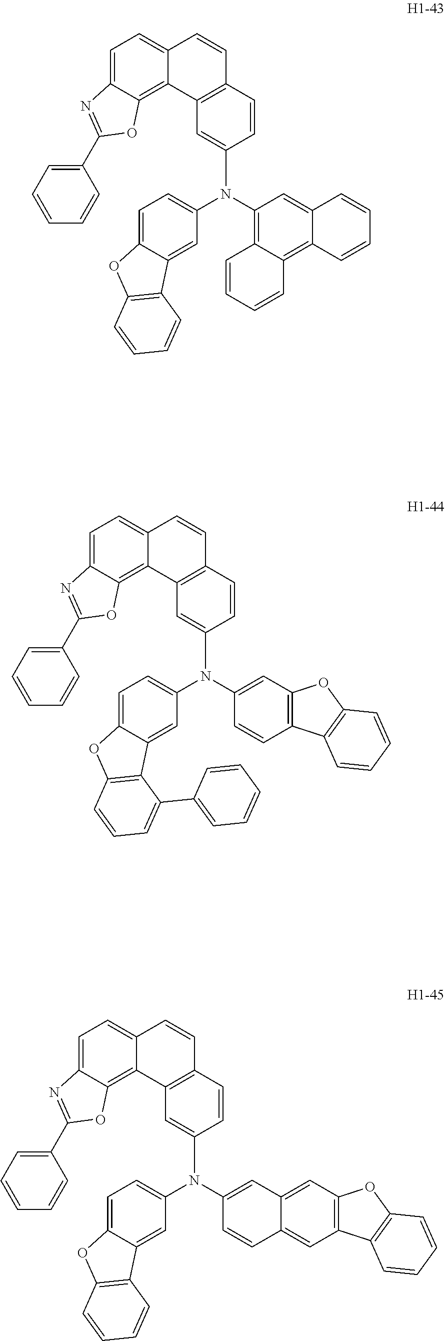 [SMILES: c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

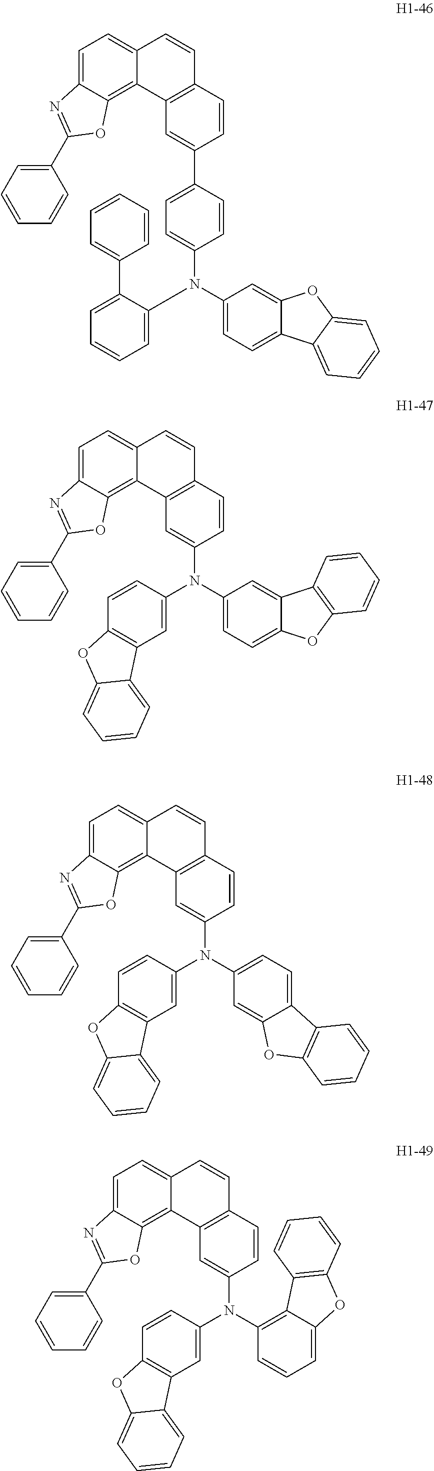 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1]

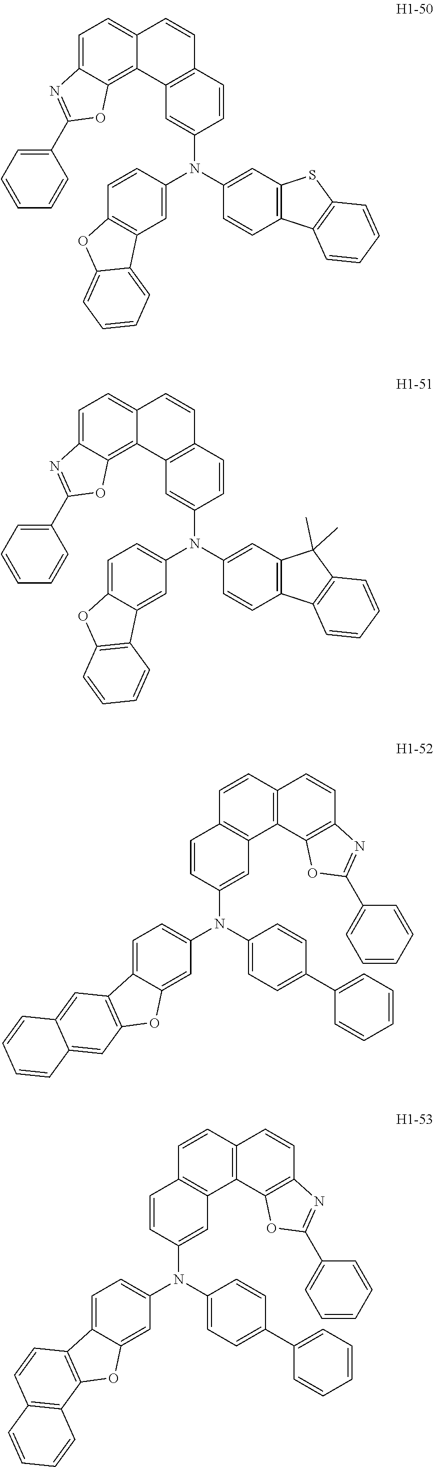 [SMILES: c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1]

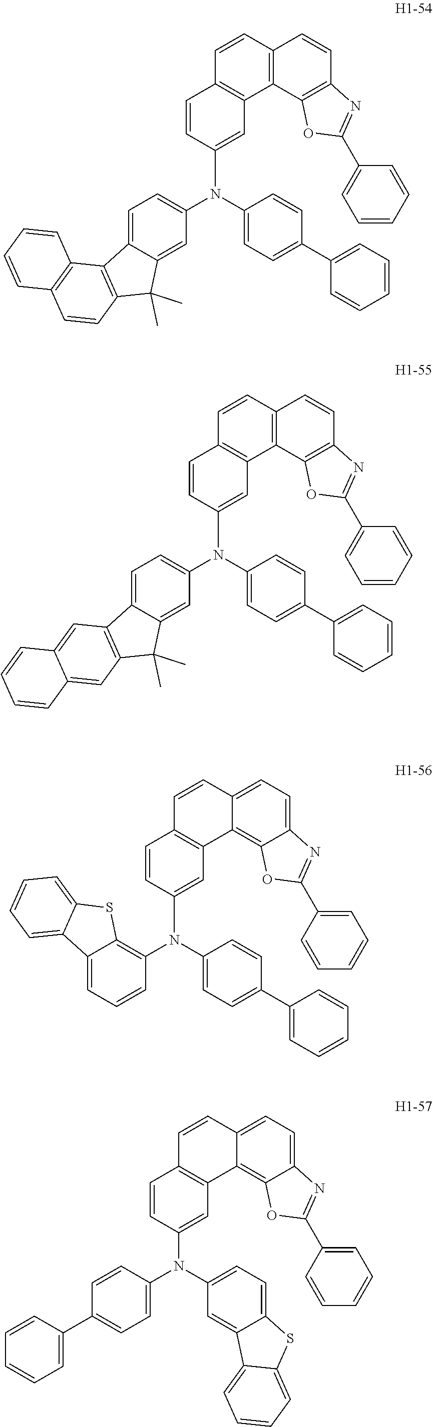 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

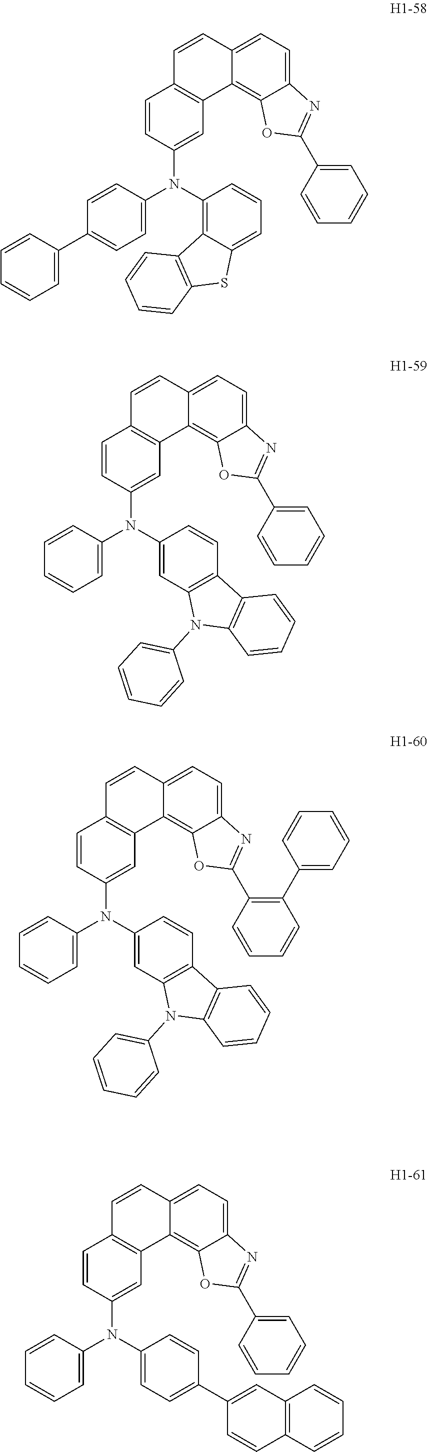 [SMILES: c1ccc(-c2ccc(N(c3ccc4ccc5ccc6nc(-c7ccccc7)oc6c5c4c3)c3cccc4sc5ccccc5c34)cc2)cc1 ; c1ccc(-c2nc3ccc4ccc5ccc(N(c6ccccc6)c6ccc7c8ccccc8n(-c8ccccc8)c7c6)cc5c4c3o2)cc1 ; c1ccc(-c2ccccc2-c2nc3ccc4ccc5ccc(N(c6ccccc6)c6ccc7c8ccccc8n(-c8ccccc8)c7c6)cc5c4c3o2)cc1 ; c1ccc(-c2nc3ccc4ccc5ccc(N(c6ccccc6)c6ccc(-c7ccc8ccccc8c7)cc6)cc5c4c3o2)cc1]

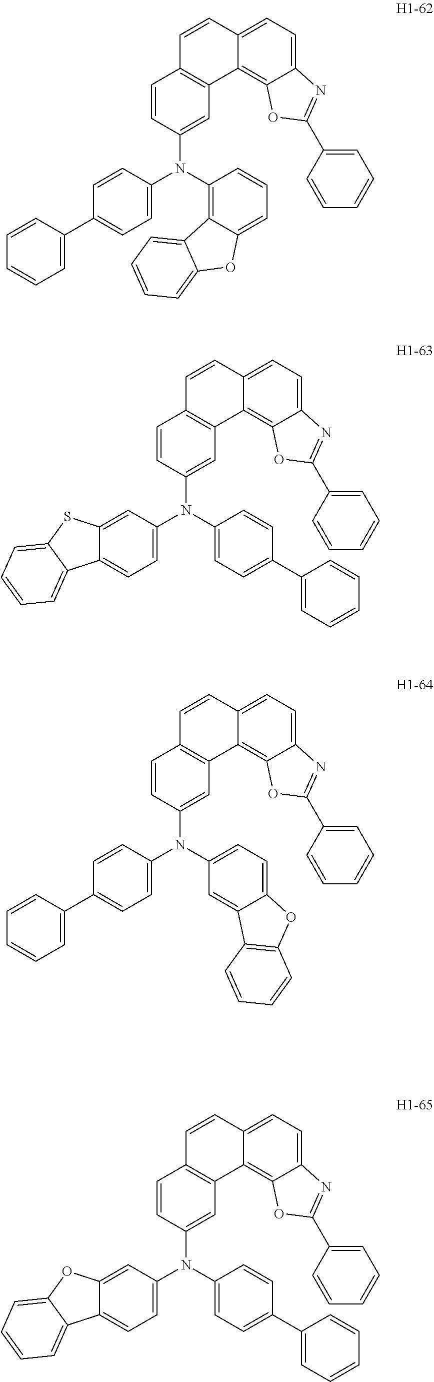 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1]

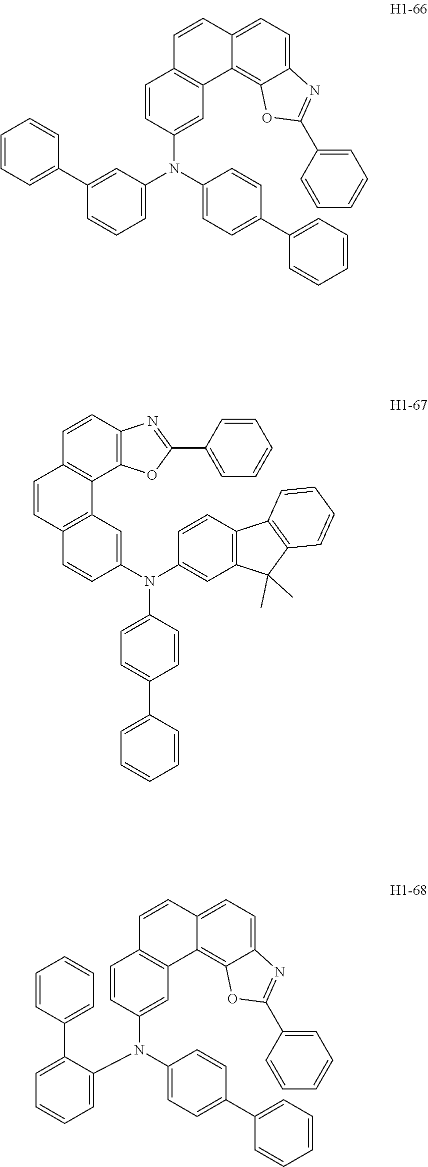 [SMILES: c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

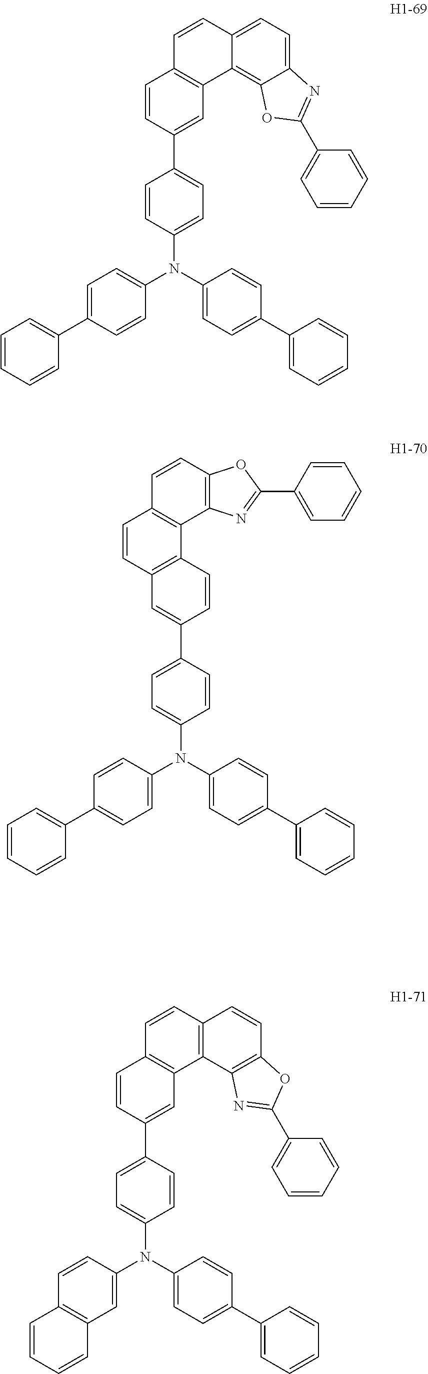 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

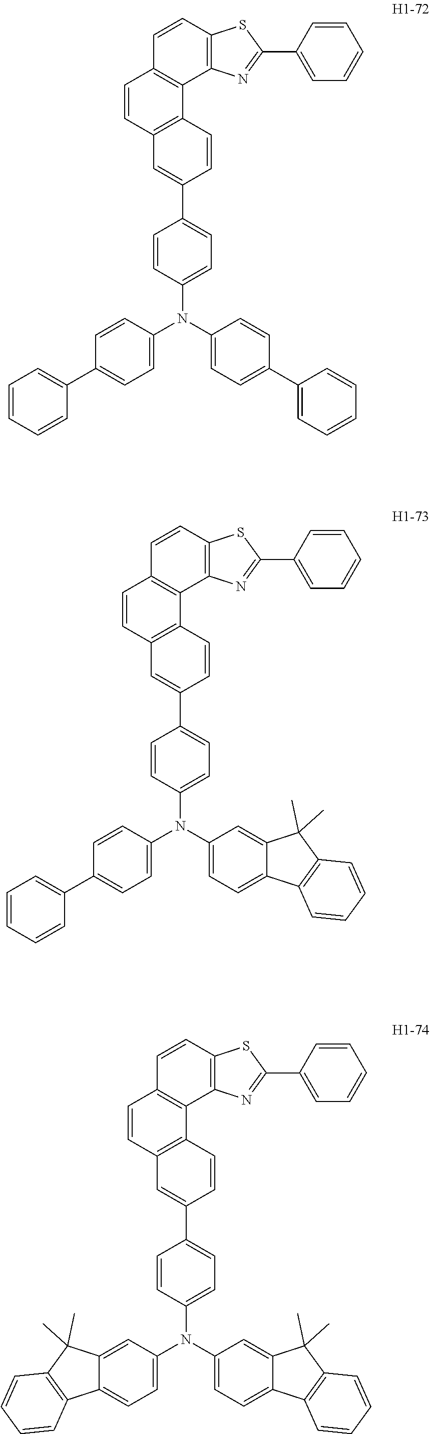 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

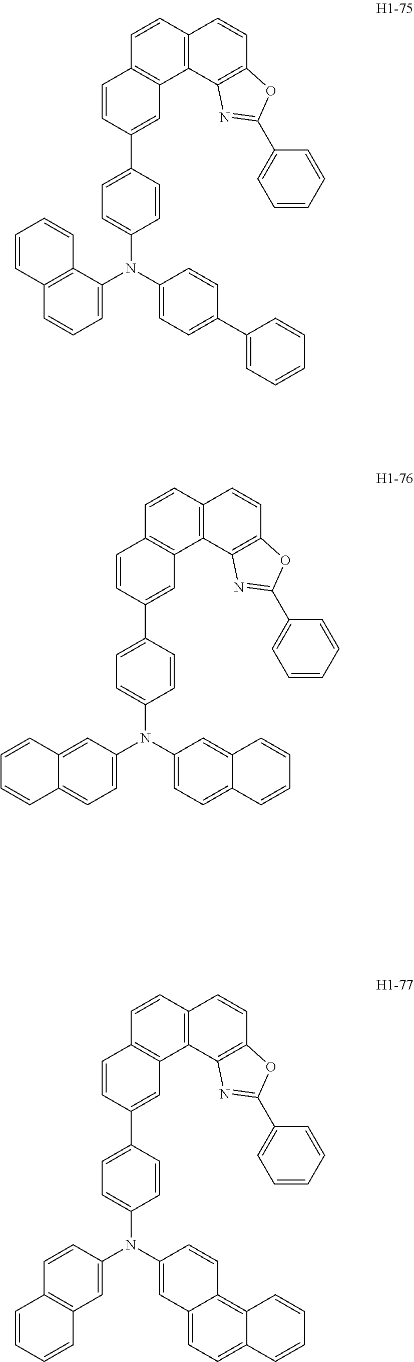 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

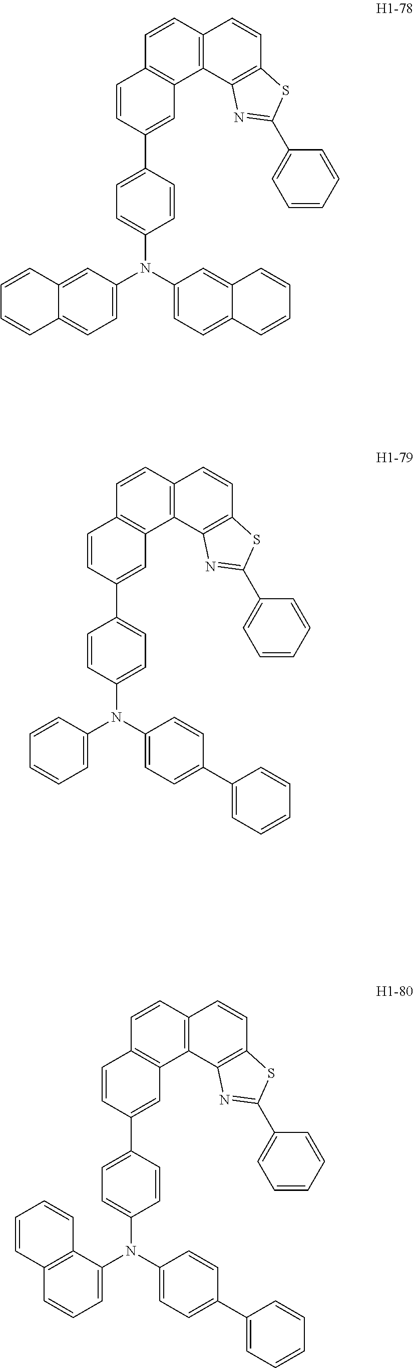 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

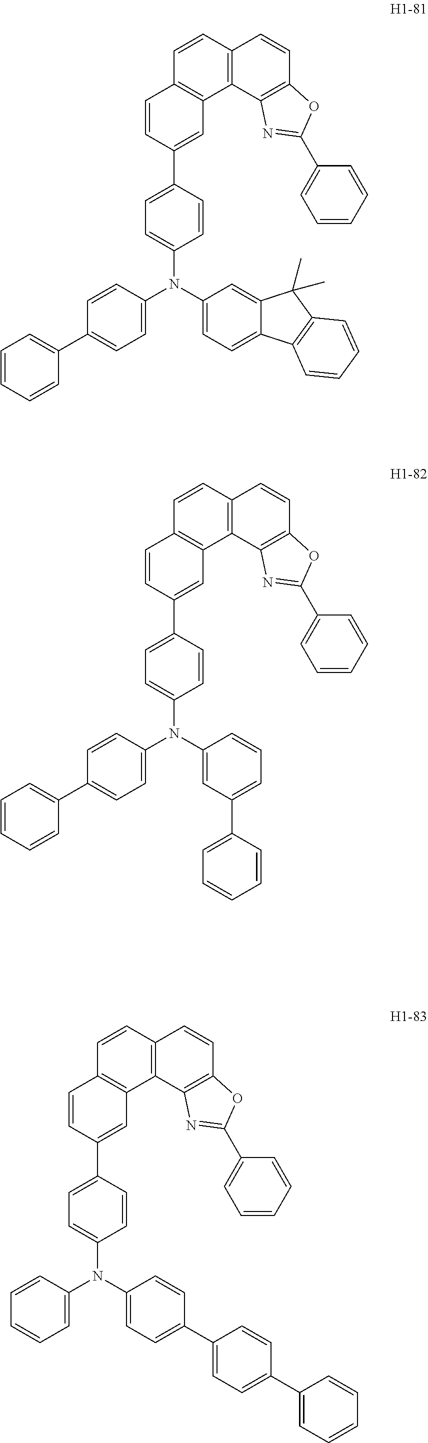 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

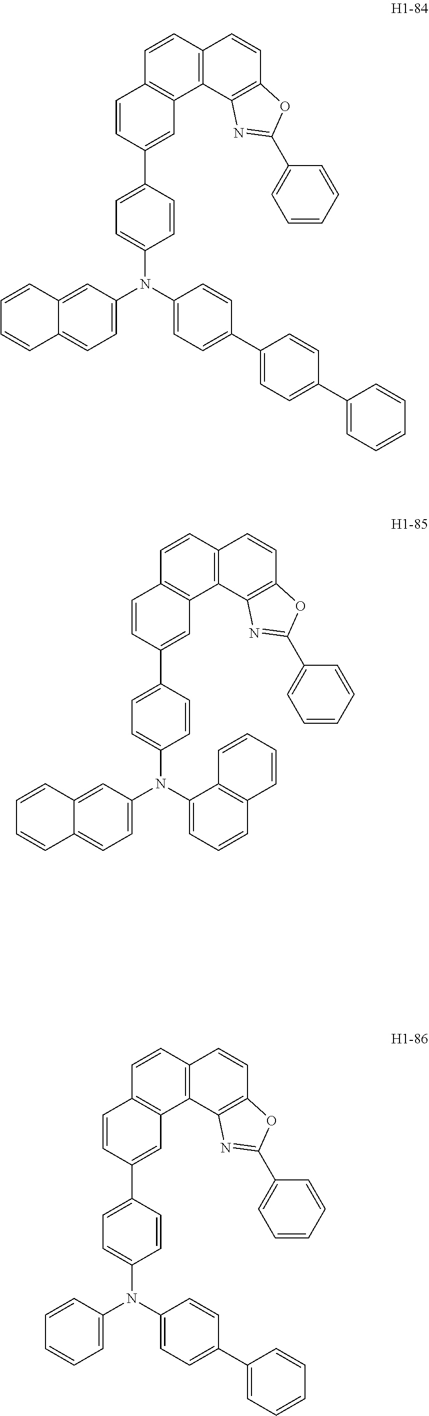 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

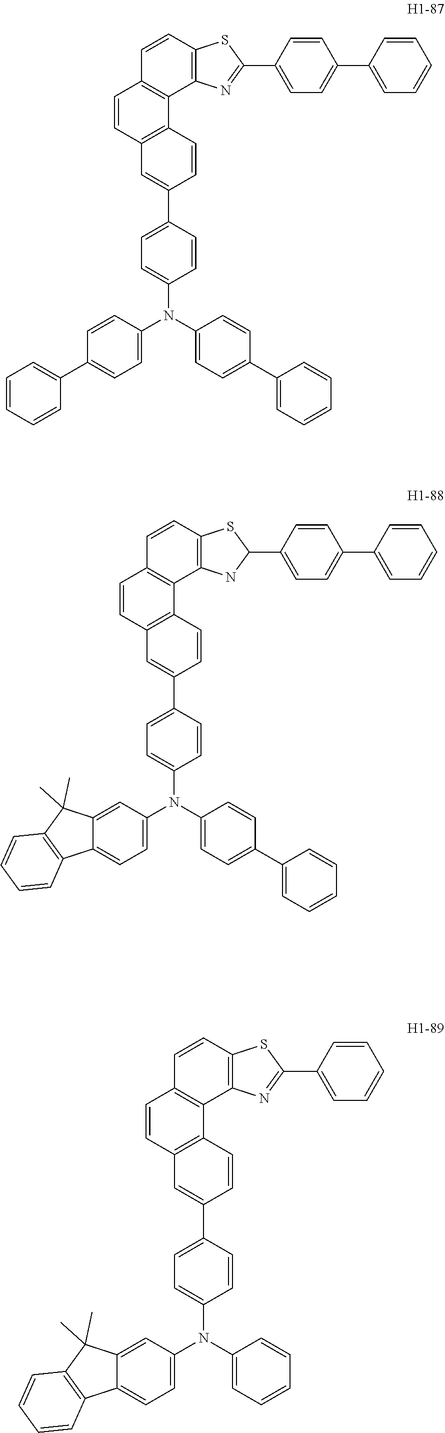

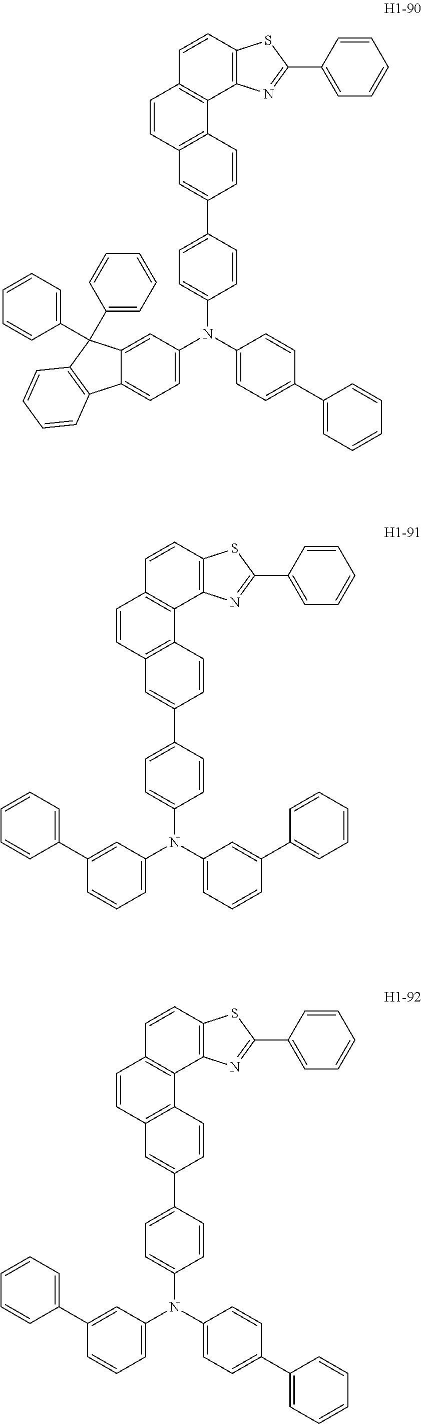 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

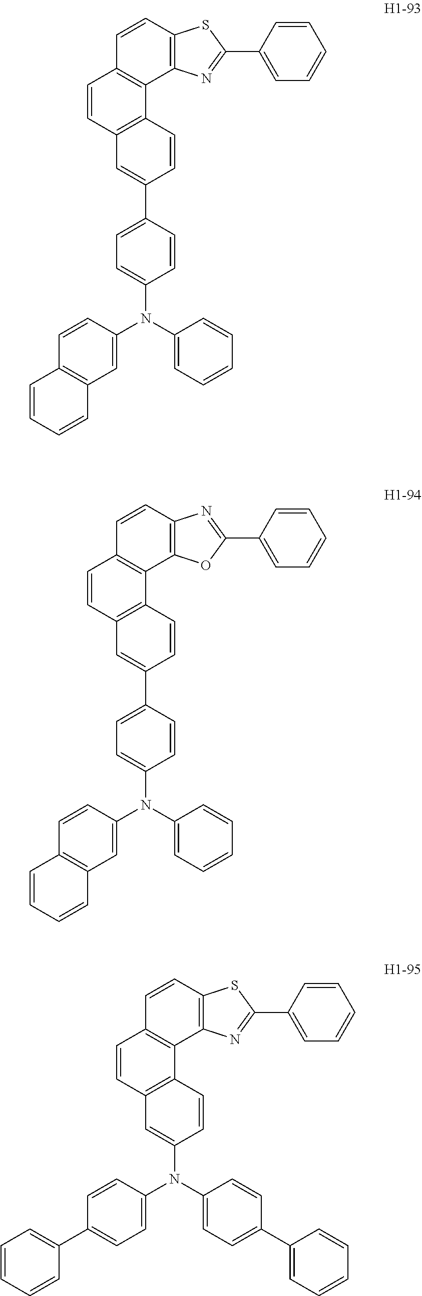 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

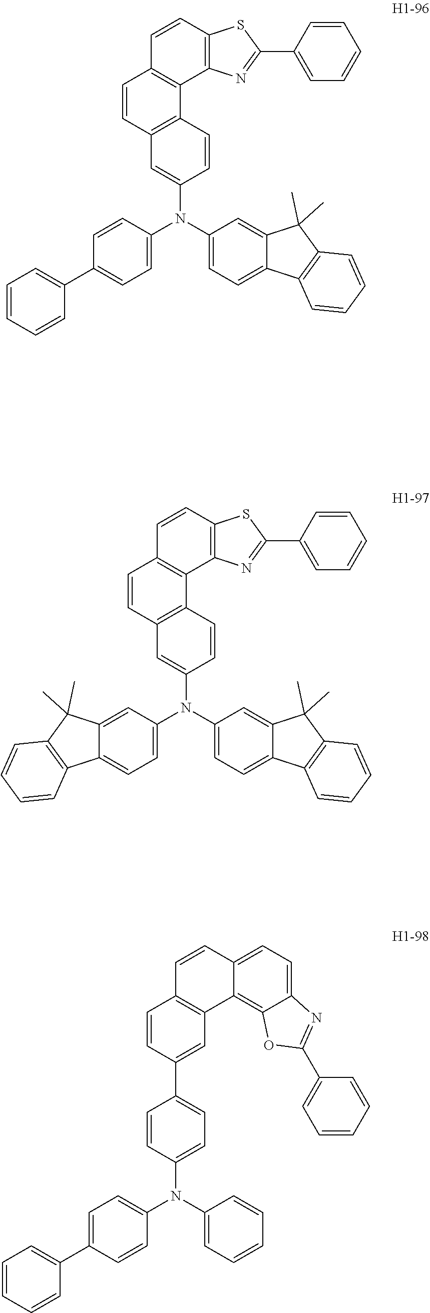 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

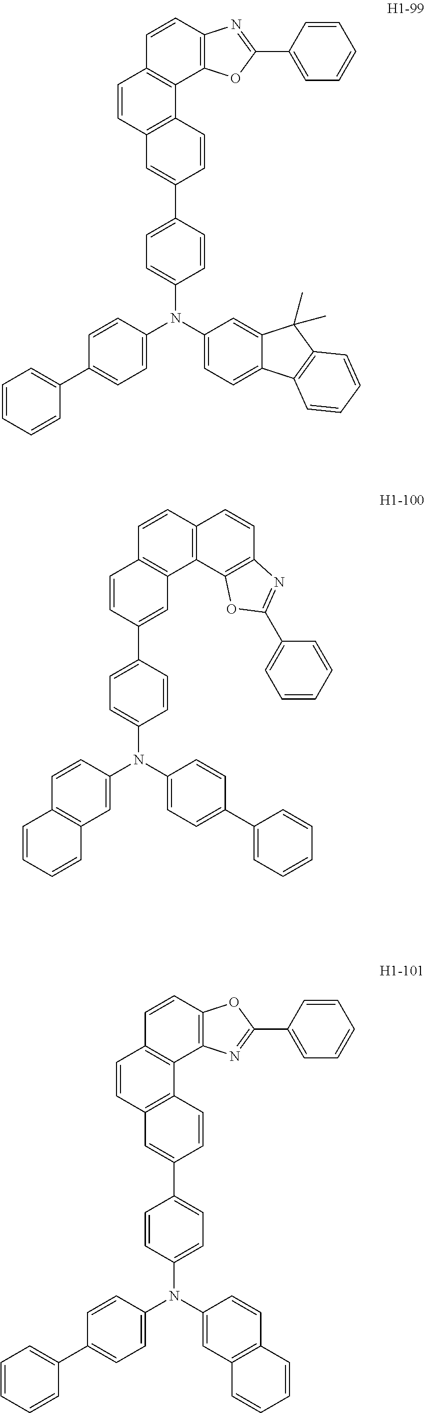 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

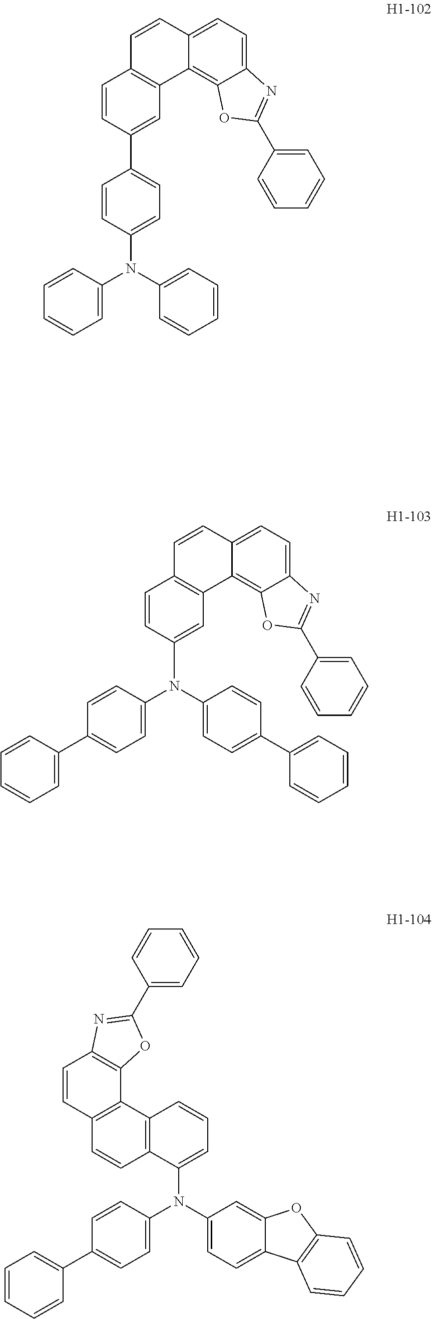 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1]

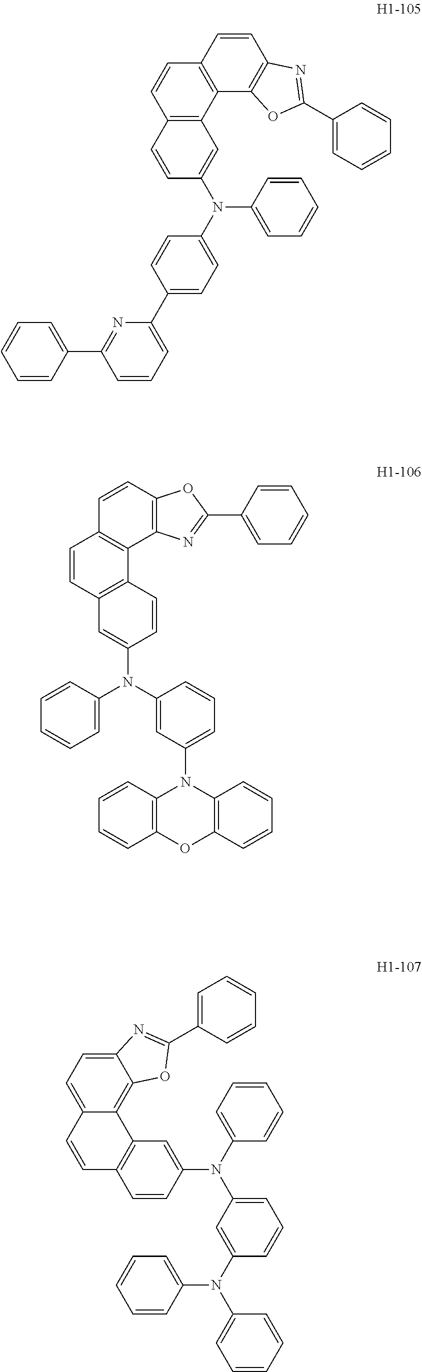 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

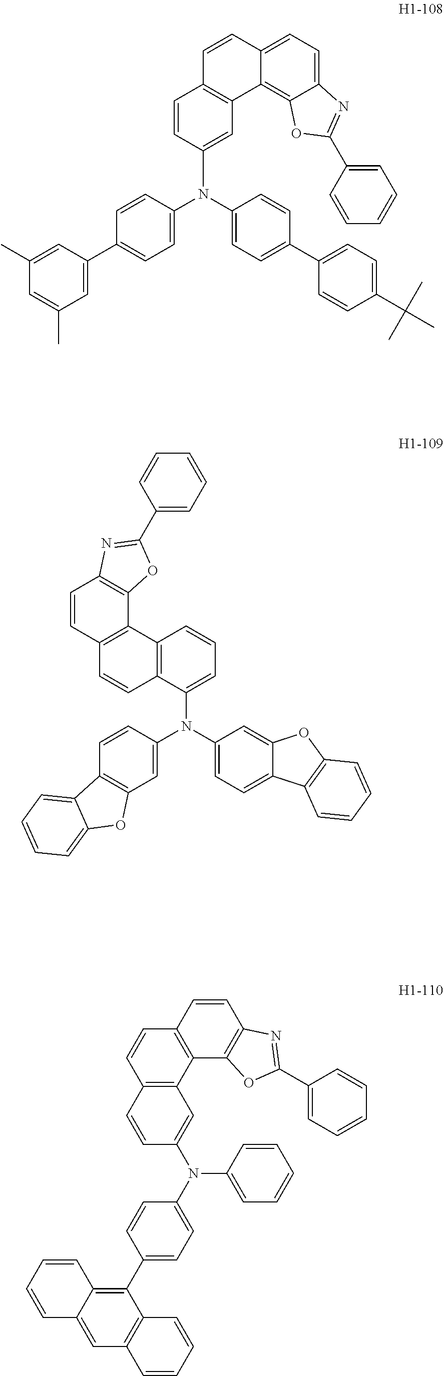 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1]

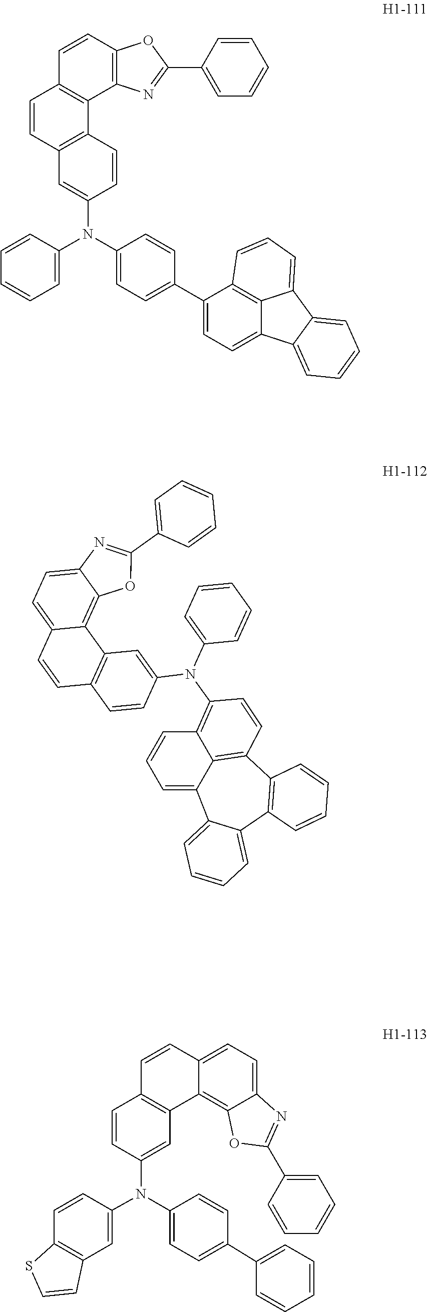

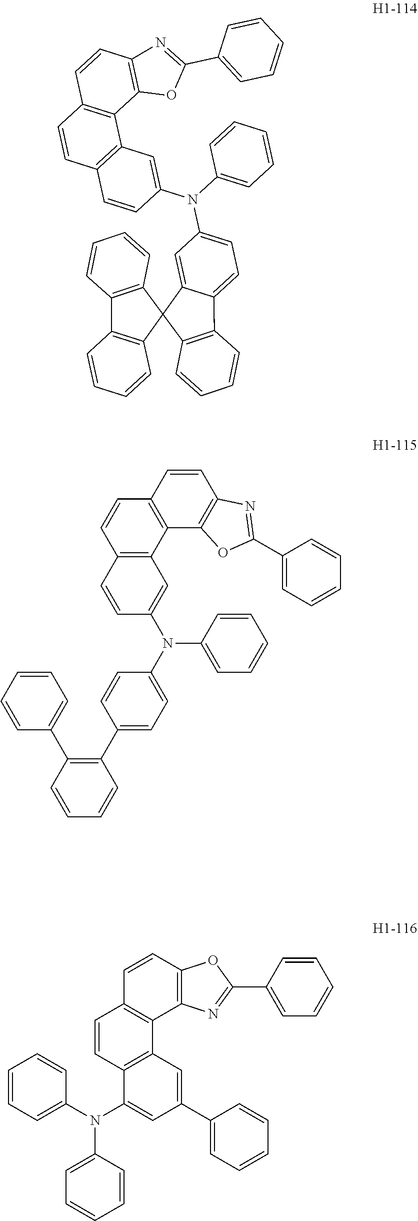 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1]

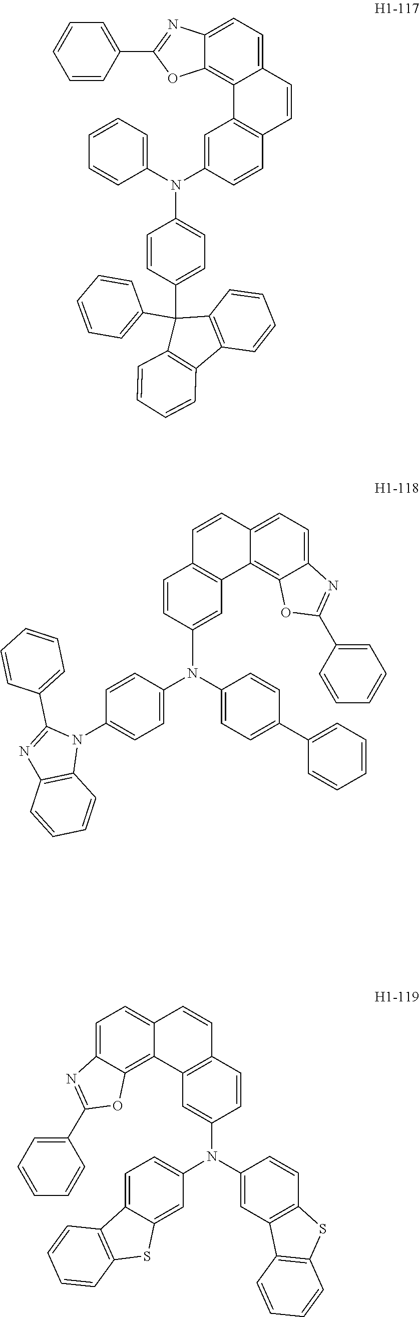 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

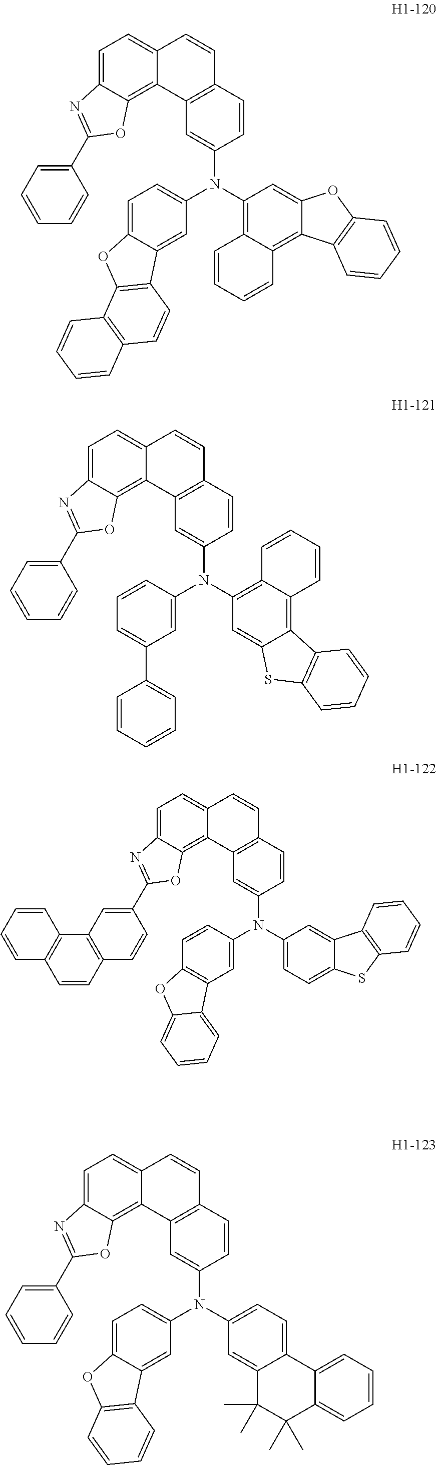 [SMILES: c1ccc(-c2nc3ccc4ccc5ccc(N(c6ccc7oc8c9ccccc9ccc8c7c6)c6cc7oc8ccccc8c7c7ccccc67)cc5c4c3o2)cc1 ; c1ccc(-c2cccc(N(c3ccc4ccc5ccc6nc(-c7ccccc7)oc6c5c4c3)c3cc4sc5ccccc5c4c4ccccc34)c2)cc1 ; c1ccc2c(c1)ccc1ccc(-c3nc4ccc5ccc6ccc(N(c7ccc8oc9ccccc9c8c7)c7ccc8sc9ccccc9c8c7)cc6c5c4o3)cc12 ; CC1(C)c2ccccc2-c2ccc(N(c3ccc4oc5ccccc5c4c3)c3ccc4ccc5ccc6nc(-c7ccccc7)oc6c5c4c3)cc2C1(C)C]

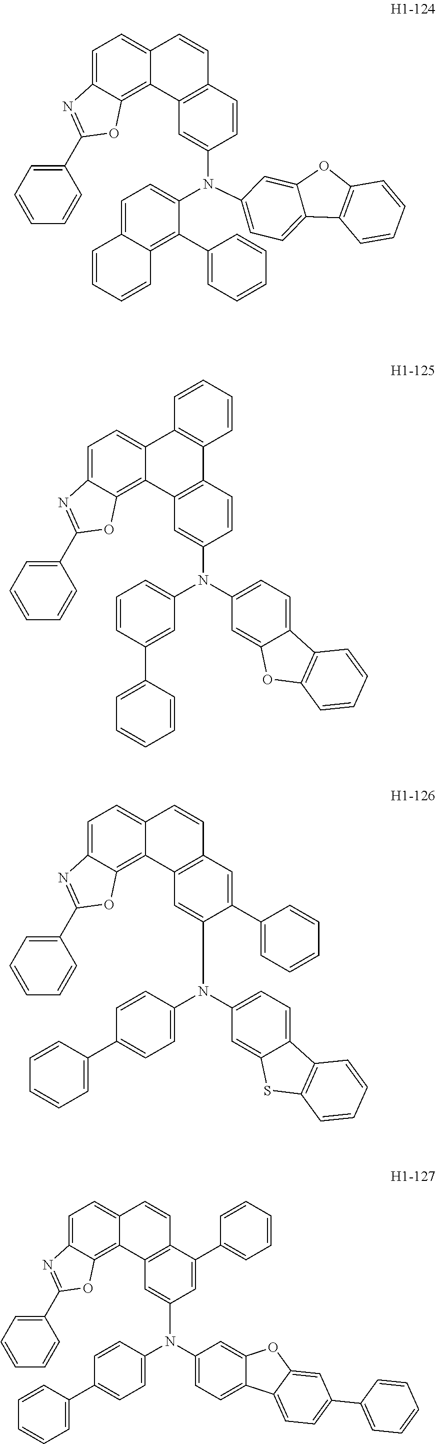 [SMILES: c1ccc(-c2nc3ccc4ccc5ccc(N(c6ccc7c(c6)oc6ccccc67)c6ccc7ccccc7c6-c6ccccc6)cc5c4c3o2)cc1 ; c1ccc(-c2cccc(N(c3ccc4c(c3)oc3ccccc34)c3ccc4c5ccccc5c5ccc6nc(-c7ccccc7)oc6c5c4c3)c2)cc1 ; c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1]

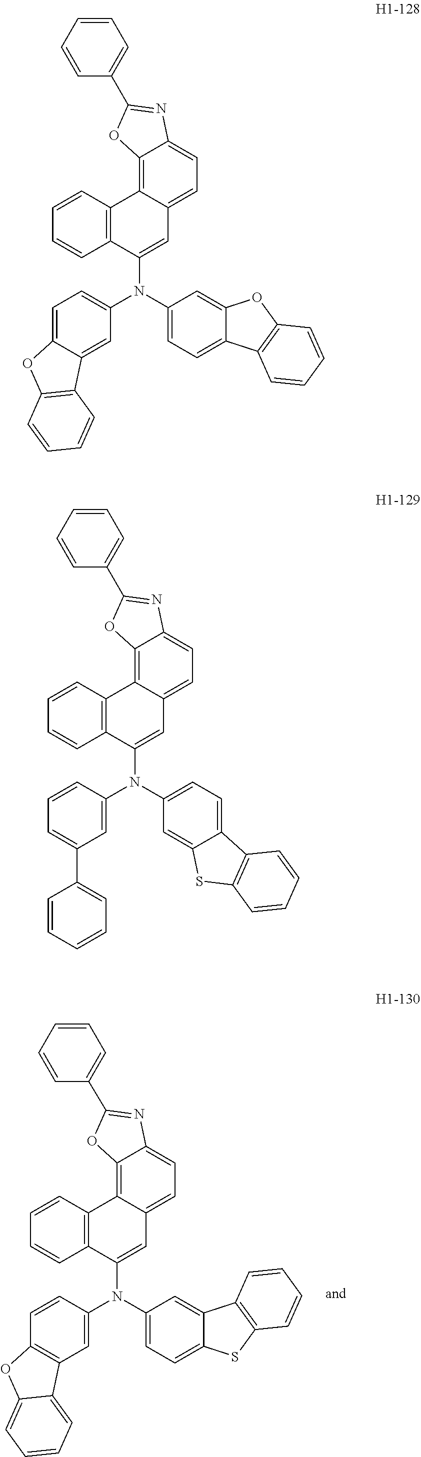 [SMILES: c1ccc(-c2nc3ccc4cc(N(c5ccc6c(c5)oc5ccccc56)c5ccc6oc7ccccc7c6c5)c5ccccc5c4c3o2)cc1 ; c1ccc(-c2cccc(N(c3ccc4c(c3)sc3ccccc34)c3cc4ccc5nc(-c6ccccc6)oc5c4c4ccccc34)c2)cc1 ; c1ccc(-c2nc3ccc4cc(N(c5ccc6oc7ccccc7c6c5)c5ccc6sc7ccccc7c6c5)c5ccccc5c4c3o2)cc1]

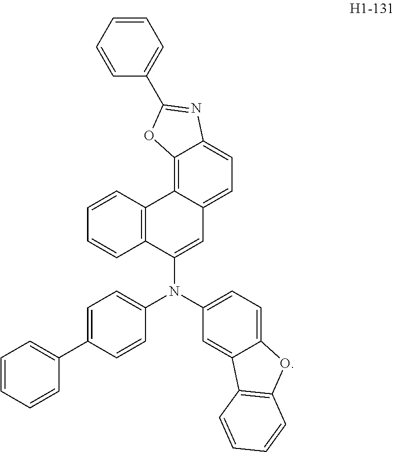 [SMILES: c1ccc(-c2ccc(N(c3ccc4oc5ccccc5c4c3)c3cc4ccc5nc(-c6ccccc6)oc5c4c4ccccc34)cc2)cc1]

7. An organic electroluminescent device comprising the plurality of host materials according to claim 1 in a light-emitting layer.

8. A compound represented by the following Formula 1: 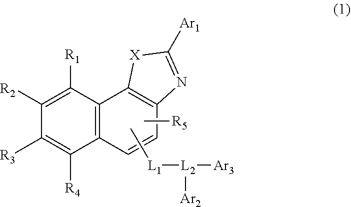
wherein,
X represents —O— or —S—;
R₁ to R₅ each independently represent, hydrogen, deuterium, halogen, cyano, a substituted or unsubstituted (C1-C30)alkyl, a substituted or unsubstituted (C3-C30)cycloalkyl, a substituted or unsubstituted (C6-C30)aryl, a substituted or unsubstituted (3- to- 30-membered)heteroaryl, a substituted or unsubstituted (C1-C30)alkoxy, or —NRₐR_{b};
Rₐ and R_{b} each independently represent, hydrogen, a substituted or unsubstituted (C1-C30)alkyl, a substituted or unsubstituted (C2-C30)alkenyl, a substituted or unsubstituted (C6-C30)aryl, or a substituted or unsubstituted (3- to- 30-membered)heteroaryl;
L₁ represents a single bond or a substituted or unsubstituted (C6-C30)arylene;
L₂ represents a substituted or unsubstituted nitrogen-containing (3- to- 30-membered)heteroarylene; and
Ar₁ to Ar₃ each independently represent, a substituted or unsubstituted (C6-C30)aryl or a substituted or unsubstituted (3- to- 30-membered)heteroaryl; provided that Ar₂ and Ar₃ are different from each other.

9. The compound according to claim 8, wherein the compound represented by Formula I is selected from the following compounds:

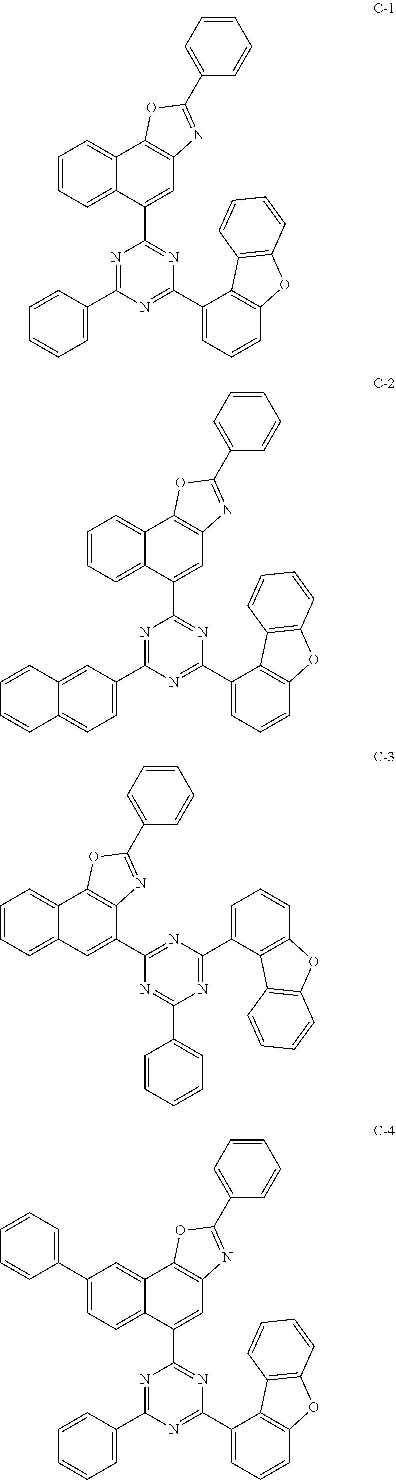

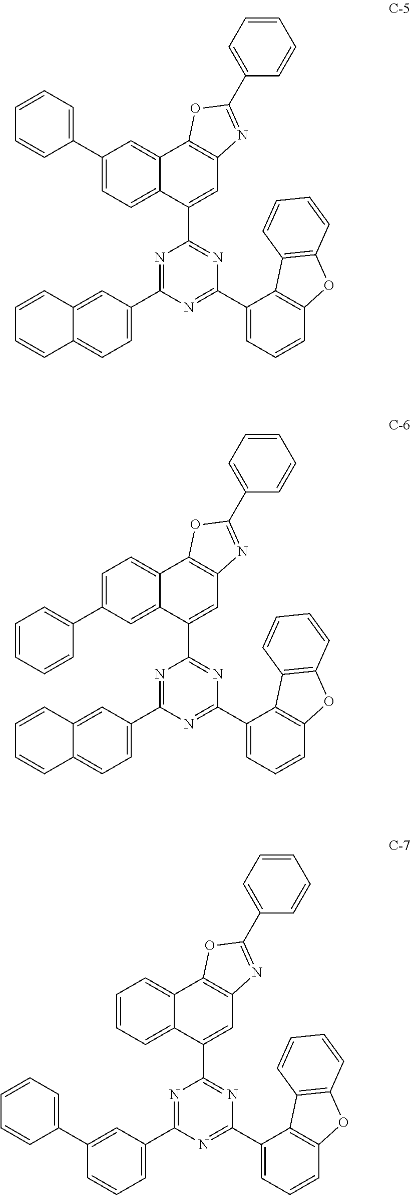 [SMILES: c1ccc(-c2ccc3c(-c4nc(-c5ccc6ccccc6c5)nc(-c5cccc6oc7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5oc6ccccc6c45)n2)cc2nc(-c4ccccc4)oc23)cc1 ; c1ccc(-c2cccc(-c3nc(-c4cc5nc(-c6ccccc6)oc5c5ccccc45)nc(-c4cccc5oc6ccccc6c45)n3)c2)cc1]

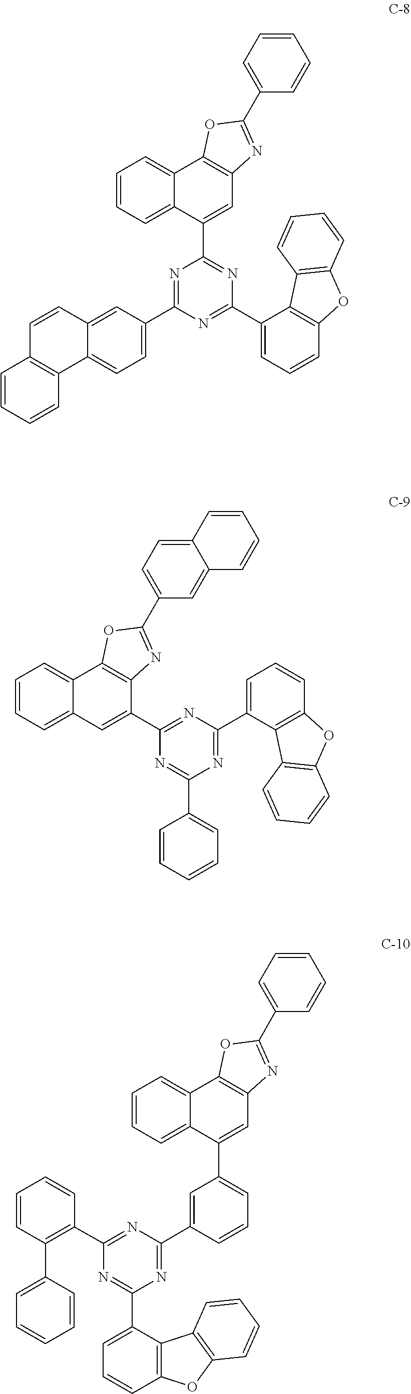 [SMILES: c1ccc(-c2nc3cc(-c4nc(-c5ccc6c(ccc7ccccc76)c5)nc(-c5cccc6oc7ccccc7c56)n4)c4ccccc4c3o2)cc1 ; c1ccc(-c2nc(-c3cc4ccccc4c4oc(-c5ccc6ccccc6c5)nc34)nc(-c3cccc4oc5ccccc5c34)n2)cc1 ; c1ccc(-c2nc3cc(-c4cccc(-c5nc(-c6ccccc6-c6ccccc6)nc(-c6cccc7oc8ccccc8c67)n5)c4)c4ccccc4c3o2)cc1]

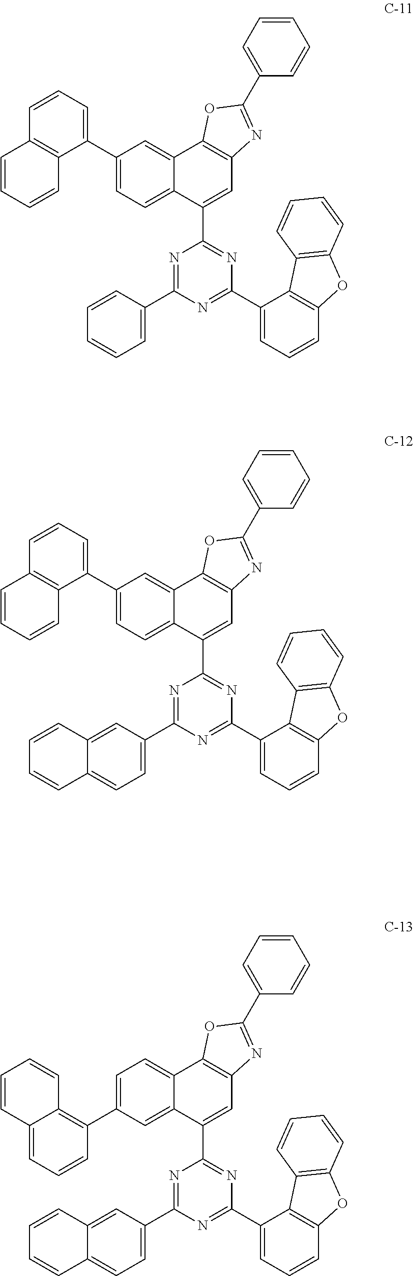 [SMILES: c1ccc(-c2nc(-c3cc4nc(-c5ccccc5)oc4c4cc(-c5cccc6ccccc56)ccc34)nc(-c3cccc4oc5ccccc5c34)n2)cc1 ; c1ccc(-c2nc3cc(-c4nc(-c5ccc6ccccc6c5)nc(-c5cccc6oc7ccccc7c56)n4)c4ccc(-c5cccc6ccccc56)cc4c3o2)cc1 ; c1ccc(-c2nc3cc(-c4nc(-c5ccc6ccccc6c5)nc(-c5cccc6oc7ccccc7c56)n4)c4cc(-c5cccc6ccccc56)ccc4c3o2)cc1]

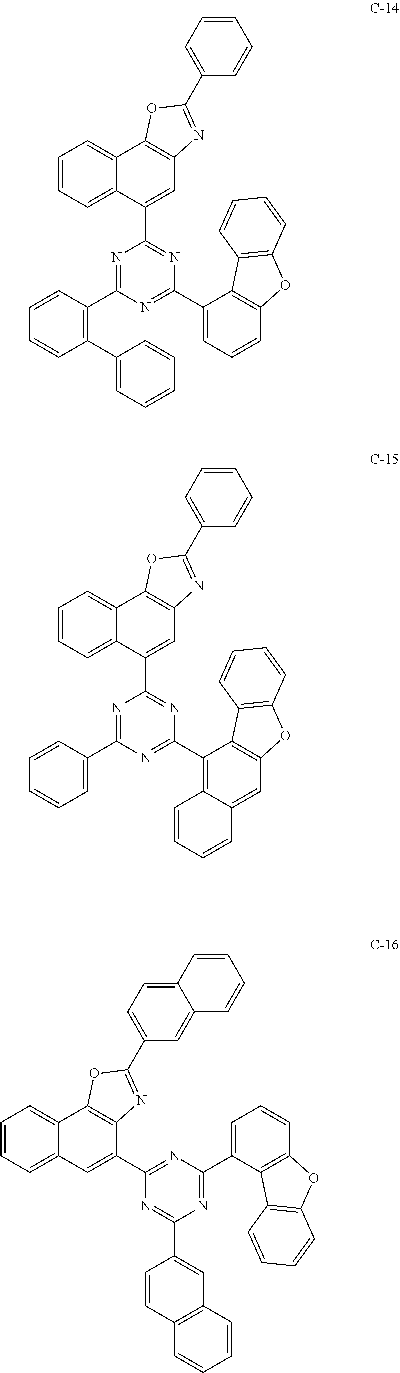 [SMILES: c1ccc(-c2nc3cc(-c4nc(-c5ccccc5-c5ccccc5)nc(-c5cccc6oc7ccccc7c56)n4)c4ccccc4c3o2)cc1 ; c1ccc(-c2nc(-c3cc4nc(-c5ccccc5)oc4c4ccccc34)nc(-c3c4ccccc4cc4oc5ccccc5c34)n2)cc1 ; c1ccc2cc(-c3nc(-c4cc5ccccc5c5oc(-c6ccc7ccccc7c6)nc45)nc(-c4cccc5oc6ccccc6c45)n3)ccc2c1]

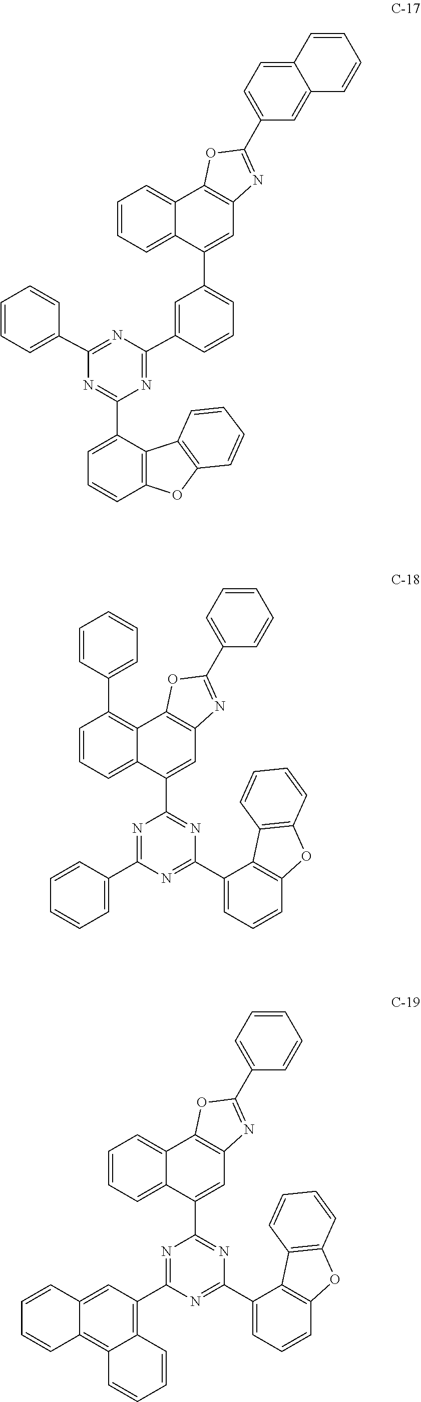 [SMILES: c1ccc(-c2nc(-c3cccc(-c4cc5nc(-c6ccc7ccccc7c6)oc5c5ccccc45)c3)nc(-c3cccc4oc5ccccc5c34)n2)cc1 ; c1ccc(-c2nc(-c3cc4nc(-c5ccccc5)oc4c4c(-c5ccccc5)cccc34)nc(-c3cccc4oc5ccccc5c34)n2)cc1 ; c1ccc(-c2nc3cc(-c4nc(-c5cc6ccccc6c6ccccc56)nc(-c5cccc6oc7ccccc7c56)n4)c4ccccc4c3o2)cc1]

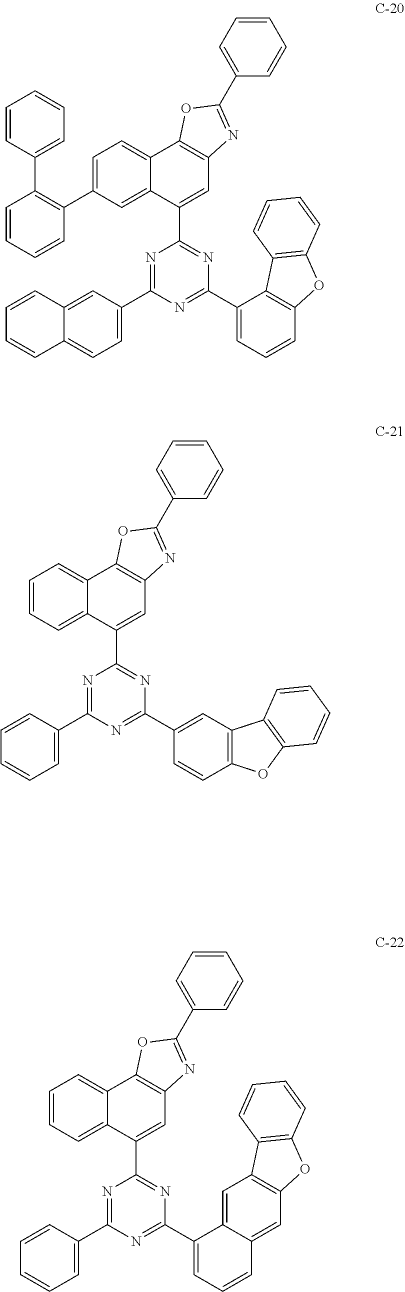 [SMILES: c1ccc(-c2nc3cc(-c4nc(-c5ccc6ccccc6c5)nc(-c5cccc6oc7ccccc7c56)n4)c4cc(-c5ccccc5-c5ccccc5)ccc4c3o2)cc1 ; c1ccc(-c2nc(-c3ccc4oc5ccccc5c4c3)nc(-c3cc4nc(-c5ccccc5)oc4c4ccccc34)n2)cc1 ; c1ccc(-c2nc(-c3cccc4cc5oc6ccccc6c5cc34)nc(-c3cc4nc(-c5ccccc5)oc4c4ccccc34)n2)cc1]

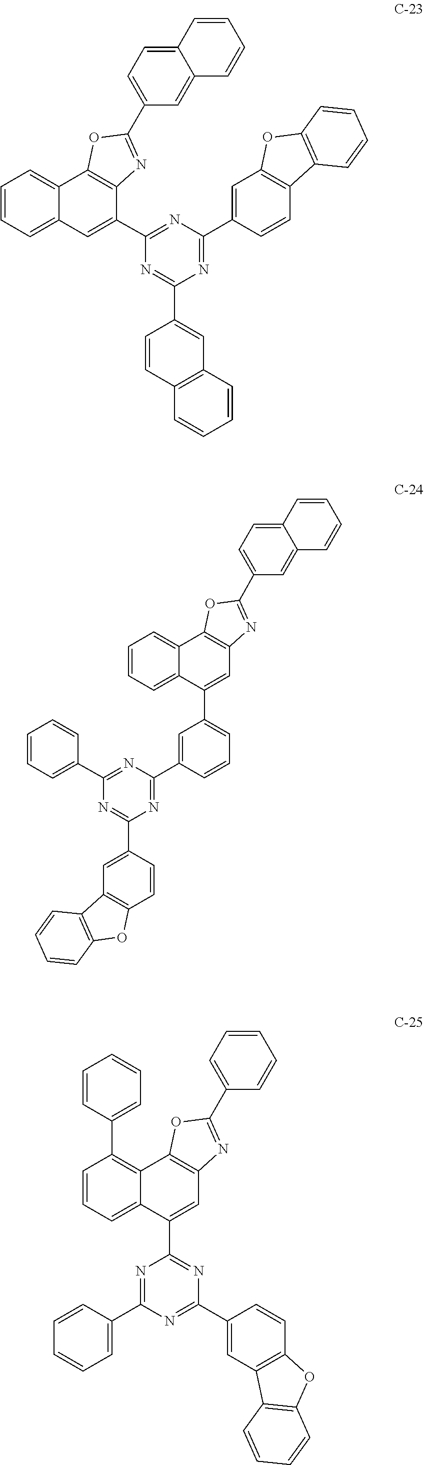 [SMILES: c1ccc2cc(-c3nc(-c4ccc5c(c4)oc4ccccc45)nc(-c4cc5ccccc5c5oc(-c6ccc7ccccc7c6)nc45)n3)ccc2c1 ; c1ccc(-c2nc(-c3cccc(-c4cc5nc(-c6ccc7ccccc7c6)oc5c5ccccc45)c3)nc(-c3ccc4oc5ccccc5c4c3)n2)cc1 ; c1ccc(-c2nc(-c3ccc4oc5ccccc5c4c3)nc(-c3cc4nc(-c5ccccc5)oc4c4c(-c5ccccc5)cccc34)n2)cc1]

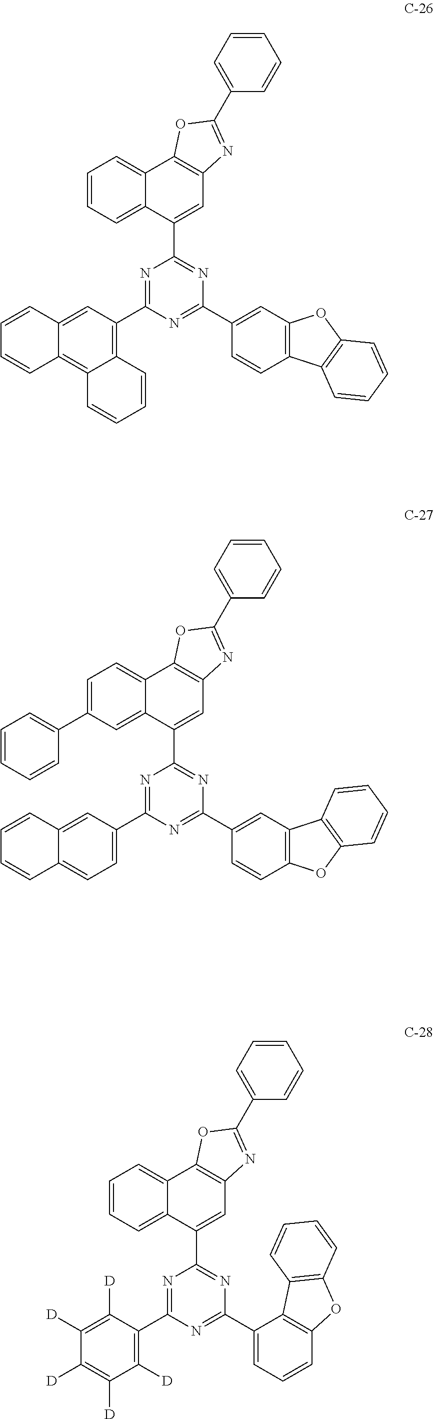 [SMILES: c1ccc(-c2nc3cc(-c4nc(-c5ccc6c(c5)oc5ccccc56)nc(-c5cc6ccccc6c6ccccc56)n4)c4ccccc4c3o2)cc1 ; c1ccc(-c2ccc3c(c2)c(-c2nc(-c4ccc5ccccc5c4)nc(-c4ccc5oc6ccccc6c5c4)n2)cc2nc(-c4ccccc4)oc23)cc1 ; [2H]c1c([2H])c([2H])c(-c2nc(-c3cc4nc(-c5ccccc5)oc4c4ccccc34)nc(-c3cccc4oc5ccccc5c34)n2)c([2H])c1[2H]]

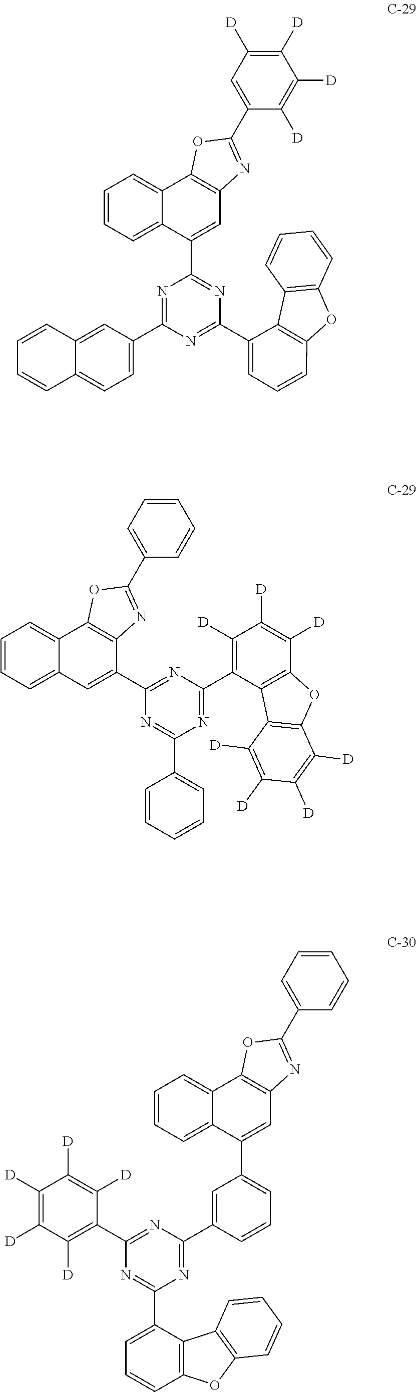 [SMILES: [2H]c1cc(-c2nc3cc(-c4nc(-c5ccc6ccccc6c5)nc(-c5cccc6oc7ccccc7c56)n4)c4ccccc4c3o2)c([2H])c([2H])c1[2H] ; [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H] ; [2H]c1c([2H])c([2H])c(-c2nc(-c3cccc(-c4cc5nc(-c6ccccc6)oc5c5ccccc45)c3)nc(-c3cccc4oc5ccccc5c34)n2)c([2H])c1[2H]]

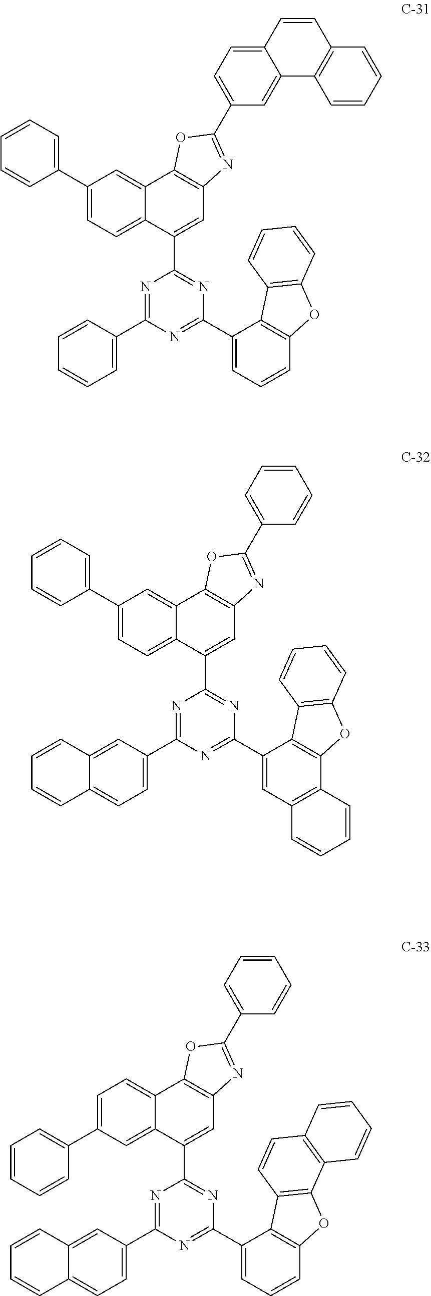 [SMILES: c1ccc(-c2ccc3c(-c4nc(-c5ccccc5)nc(-c5cccc6oc7ccccc7c56)n4)cc4nc(-c5ccc6ccc7ccccc7c6c5)oc4c3c2)cc1 ; c1ccc(-c2ccc3c(-c4nc(-c5ccc6ccccc6c5)nc(-c5cc6ccccc6c6oc7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5oc6c7ccccc7ccc6c45)n2)cc2nc(-c4ccccc4)oc23)cc1]

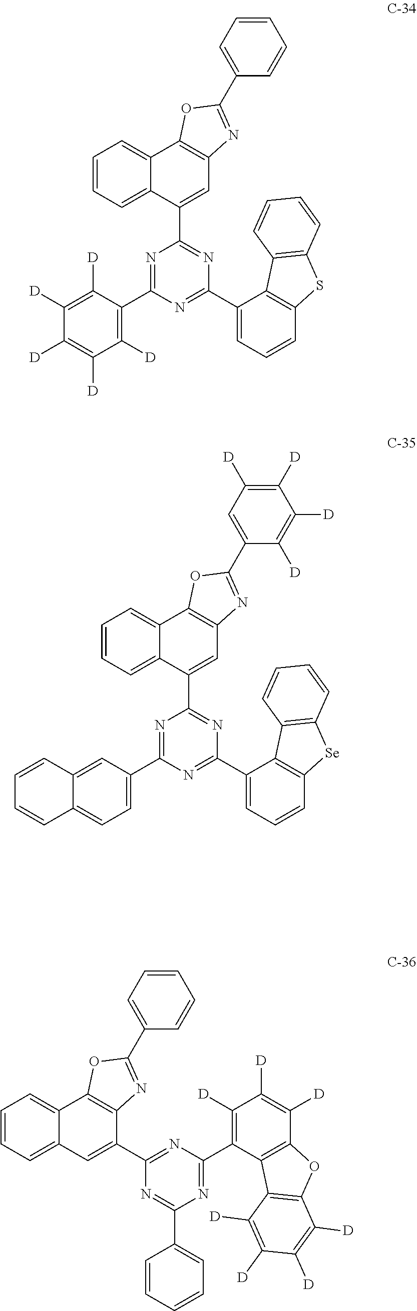 [SMILES: [2H]c1c([2H])c([2H])c(-c2nc(-c3cc4nc(-c5ccccc5)oc4c4ccccc34)nc(-c3cccc4sc5ccccc5c34)n2)c([2H])c1[2H] ; [2H]c1cc(-c2nc3cc(-c4nc(-c5ccc6ccccc6c5)nc(-c5cccc6[se]c7ccccc7c56)n4)c4ccccc4c3o2)c([2H])c([2H])c1[2H] ; [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H]]

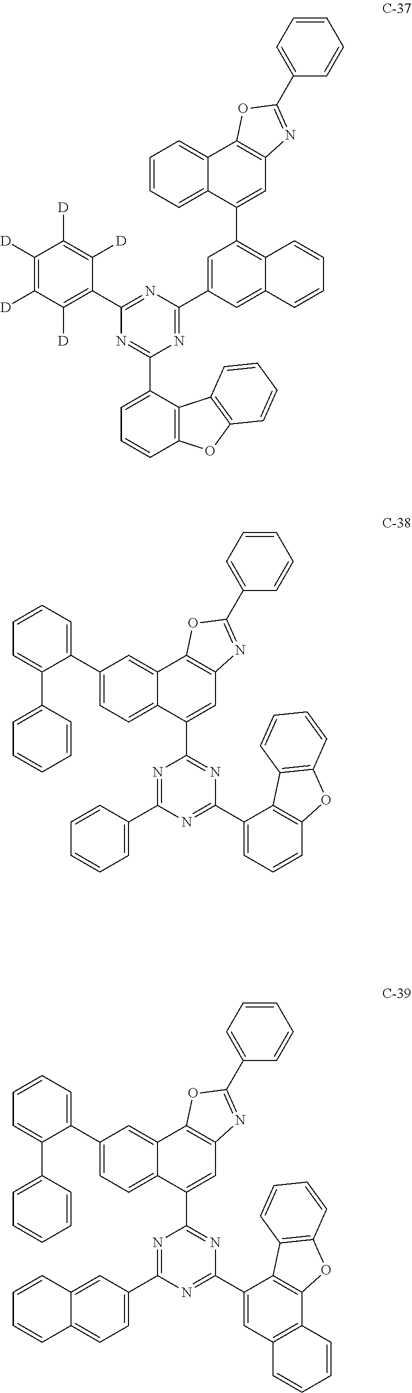 [SMILES: [2H]c1c([2H])c([2H])c(-c2nc(-c3cc(-c4cc5nc(-c6ccccc6)oc5c5ccccc45)c4ccccc4c3)nc(-c3cccc4oc5ccccc5c34)n2)c([2H])c1[2H] ; c1ccc(-c2nc(-c3cc4nc(-c5ccccc5)oc4c4cc(-c5ccccc5-c5ccccc5)ccc34)nc(-c3cccc4oc5ccccc5c34)n2)cc1 ; c1ccc(-c2nc3cc(-c4nc(-c5ccc6ccccc6c5)nc(-c5cc6ccccc6c6oc7ccccc7c56)n4)c4ccc(-c5ccccc5-c5ccccc5)cc4c3o2)cc1]

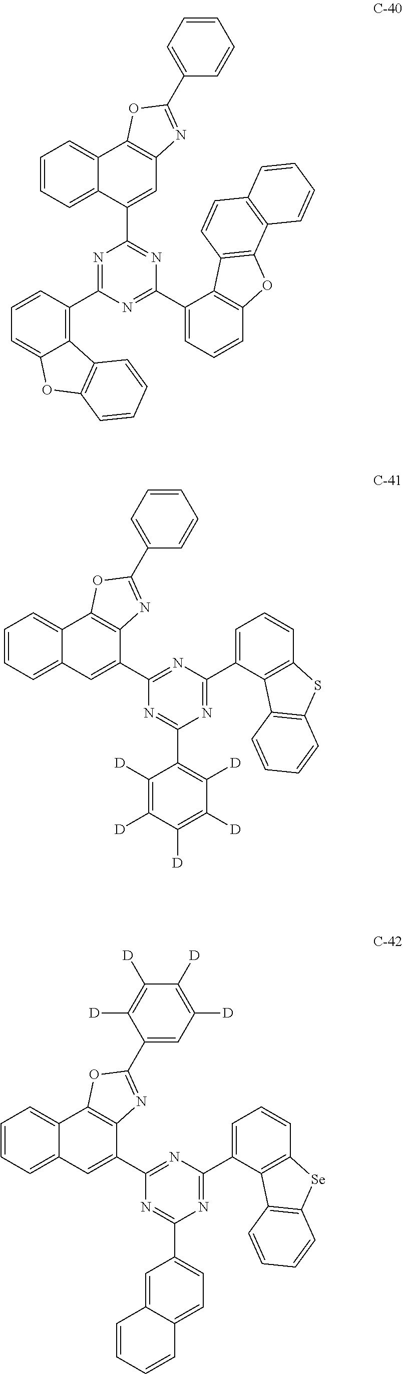 [SMILES: c1ccc(-c2nc3cc(-c4nc(-c5cccc6oc7ccccc7c56)nc(-c5cccc6oc7c8ccccc8ccc7c56)n4)c4ccccc4c3o2)cc1 ; [2H]c1c([2H])c([2H])c(-c2nc(-c3cc4ccccc4c4oc(-c5ccccc5)nc34)nc(-c3cccc4sc5ccccc5c34)n2)c([2H])c1[2H] ; [2H]c1cc(-c2nc3c(-c4nc(-c5ccc6ccccc6c5)nc(-c5cccc6[se]c7ccccc7c56)n4)cc4ccccc4c3o2)c([2H])c([2H])c1[2H]]

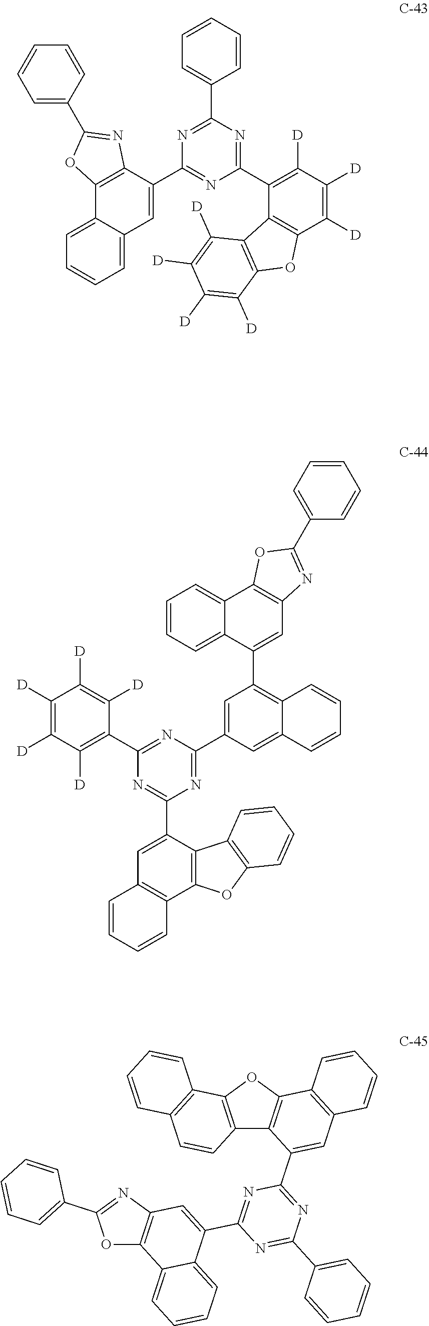 [SMILES: [2H]c1c([2H])c([2H])c2c(oc3c([2H])c([2H])c([2H])c(-c4nc(-c5ccccc5)nc(-c5cc6ccccc6c6oc(-c7ccccc7)nc56)n4)c32)c1[2H] ; [2H]c1c([2H])c([2H])c(-c2nc(-c3cc(-c4cc5nc(-c6ccccc6)oc5c5ccccc45)c4ccccc4c3)nc(-c3cc4ccccc4c4oc5ccccc5c34)n2)c([2H])c1[2H] ; c1ccc(-c2nc(-c3cc4nc(-c5ccccc5)oc4c4ccccc34)nc(-c3cc4ccccc4c4oc5c6ccccc6ccc5c34)n2)cc1]

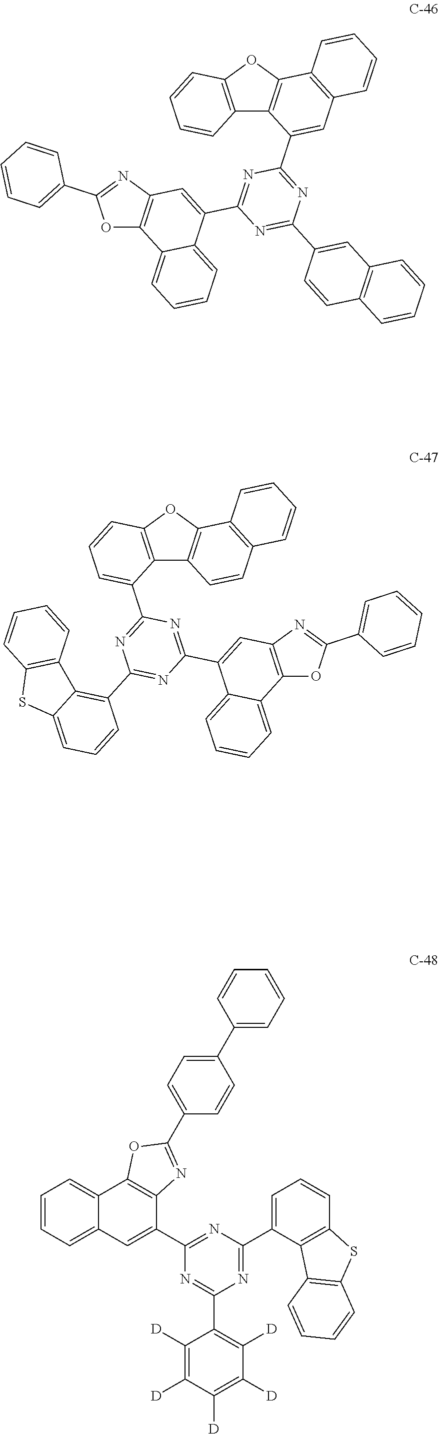 [SMILES: c1ccc(-c2nc3cc(-c4nc(-c5ccc6ccccc6c5)nc(-c5cc6ccccc6c6oc7ccccc7c56)n4)c4ccccc4c3o2)cc1 ; c1ccc(-c2nc3cc(-c4nc(-c5cccc6oc7c8ccccc8ccc7c56)nc(-c5cccc6sc7ccccc7c56)n4)c4ccccc4c3o2)cc1 ; [2H]c1c([2H])c([2H])c(-c2nc(-c3cc4ccccc4c4oc(-c5ccc(-c6ccccc6)cc5)nc34)nc(-c3cccc4sc5ccccc5c34)n2)c([2H])c1[2H]]

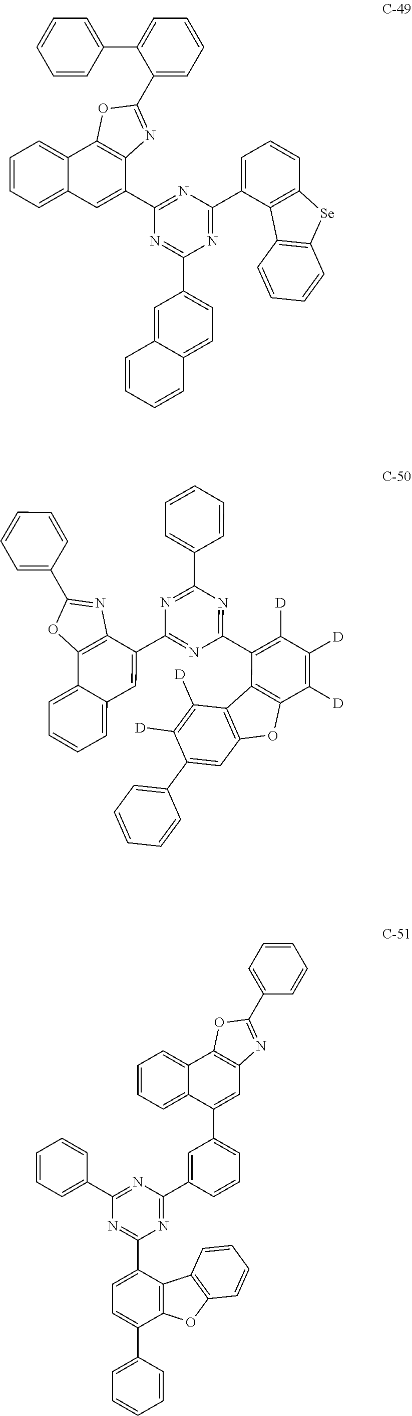 [SMILES: c1ccc(-c2ccccc2-c2nc3c(-c4nc(-c5ccc6ccccc6c5)nc(-c5cccc6[se]c7ccccc7c56)n4)cc4ccccc4c3o2)cc1 ; [2H]c1c([2H])c(-c2nc(-c3ccccc3)nc(-c3cc4ccccc4c4oc(-c5ccccc5)nc34)n2)c2c(oc3cc(-c4ccccc4)c([2H])c([2H])c32)c1[2H] ; c1ccc(-c2nc(-c3cccc(-c4cc5nc(-c6ccccc6)oc5c5ccccc45)c3)nc(-c3ccc(-c4ccccc4)c4oc5ccccc5c34)n2)cc1]

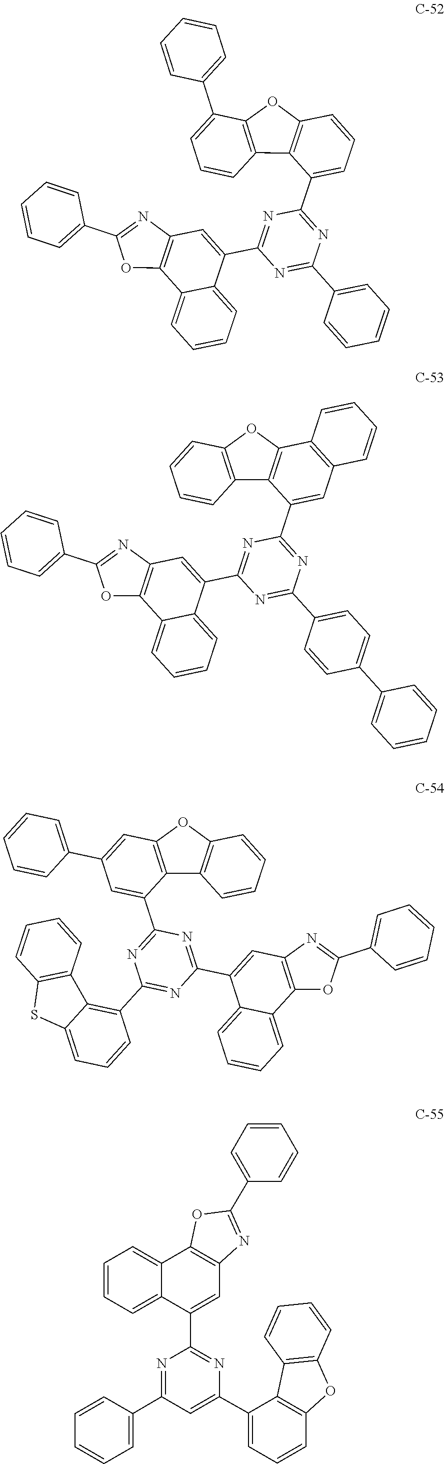 [SMILES: c1ccc(-c2nc(-c3cc4nc(-c5ccccc5)oc4c4ccccc34)nc(-c3cccc4oc5c(-c6ccccc6)cccc5c34)n2)cc1 ; c1ccc(-c2ccc(-c3nc(-c4cc5nc(-c6ccccc6)oc5c5ccccc45)nc(-c4cc5ccccc5c5oc6ccccc6c45)n3)cc2)cc1 ; c1ccc(-c2cc(-c3nc(-c4cc5nc(-c6ccccc6)oc5c5ccccc45)nc(-c4cccc5sc6ccccc6c45)n3)c3c(c2)oc2ccccc23)cc1 ; c1ccc(-c2cc(-c3cccc4oc5ccccc5c34)nc(-c3cc4nc(-c5ccccc5)oc4c4ccccc34)n2)cc1]

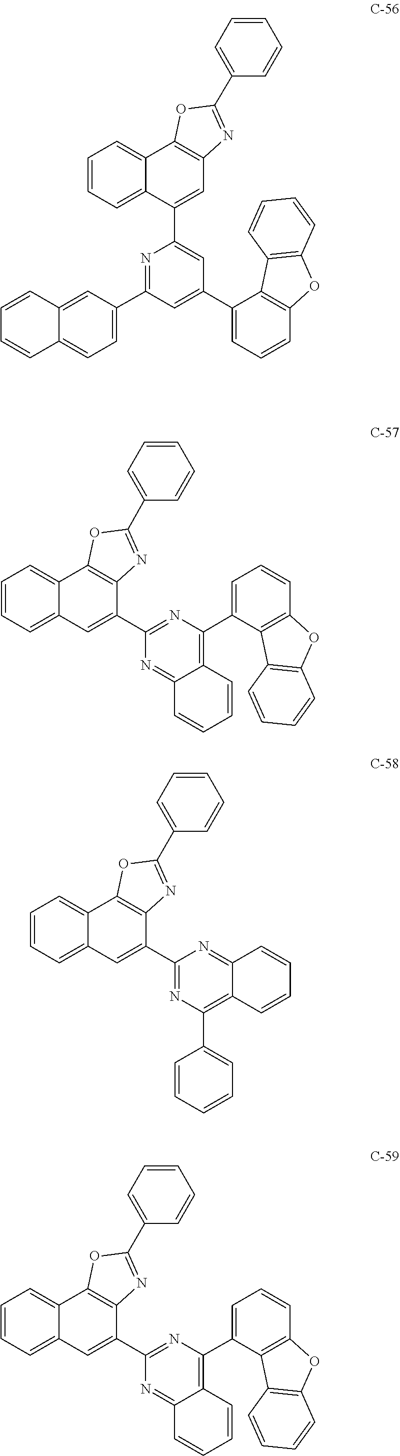

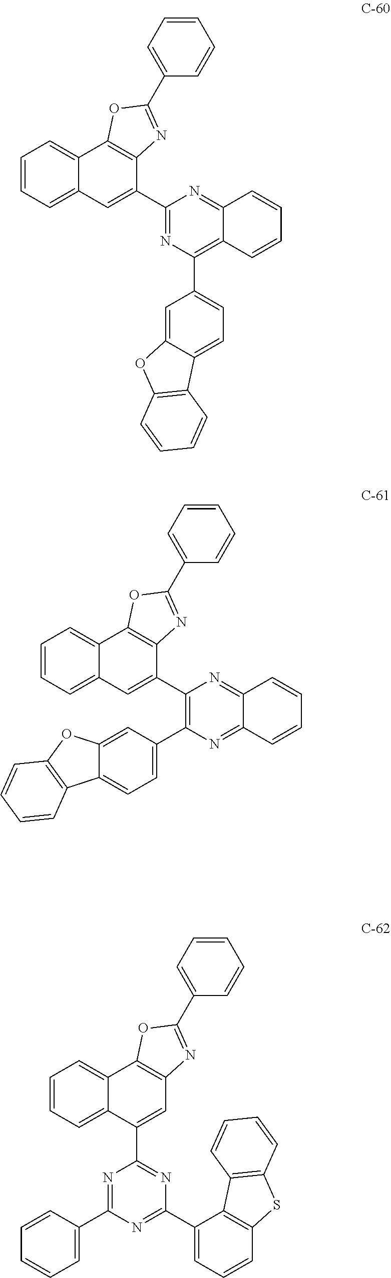 [SMILES: c1ccc(-c2nc3c(-c4nc(-c5ccc6c(c5)oc5ccccc56)c5ccccc5n4)cc4ccccc4c3o2)cc1 ; c1ccc(-c2nc3c(-c4nc5ccccc5nc4-c4ccc5c(c4)oc4ccccc45)cc4ccccc4c3o2)cc1 ; c1ccc(-c2nc(-c3cc4nc(-c5ccccc5)oc4c4ccccc34)nc(-c3cccc4sc5ccccc5c34)n2)cc1]

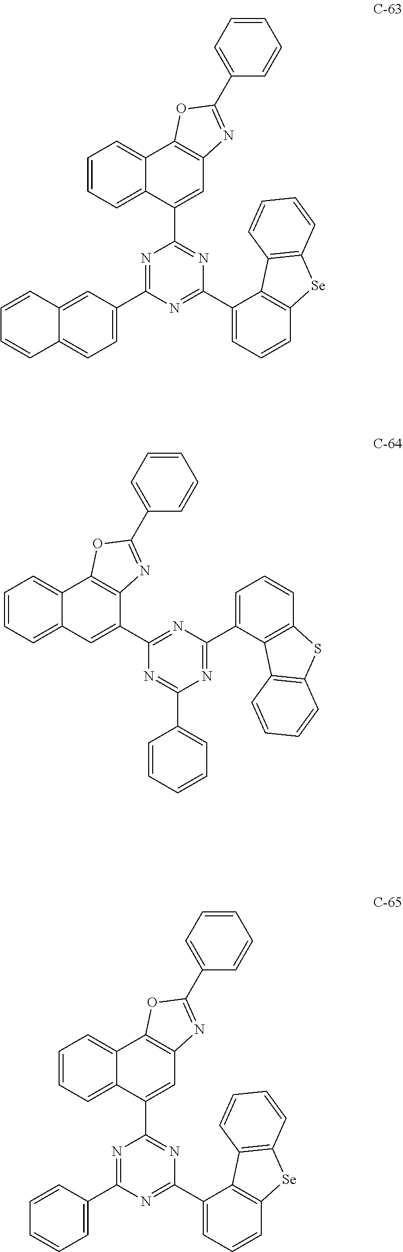 [SMILES: c1ccc(-c2nc3cc(-c4nc(-c5ccc6ccccc6c5)nc(-c5cccc6[se]c7ccccc7c56)n4)c4ccccc4c3o2)cc1 ; c1ccc(-c2nc(-c3cc4ccccc4c4oc(-c5ccccc5)nc34)nc(-c3cccc4sc5ccccc5c34)n2)cc1 ; c1ccc(-c2nc(-c3cc4nc(-c5ccccc5)oc4c4ccccc34)nc(-c3cccc4[se]c5ccccc5c34)n2)cc1]

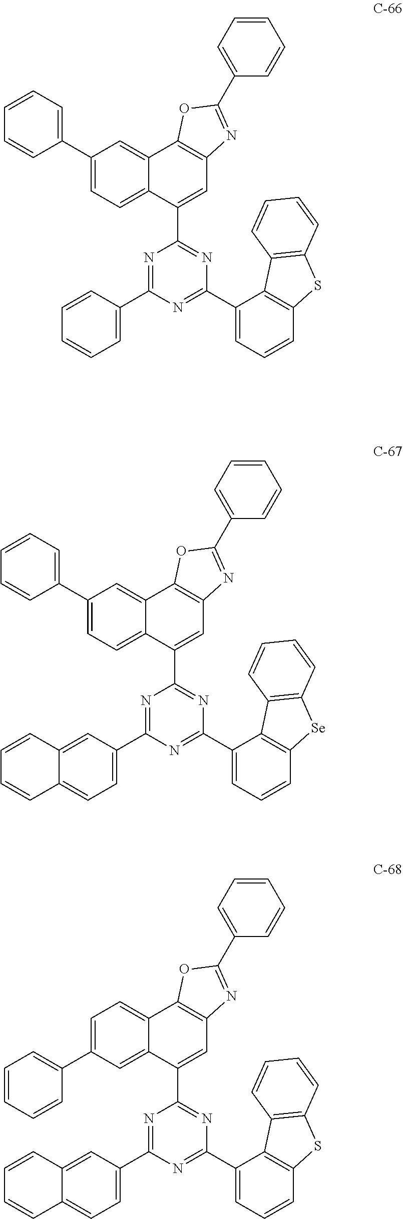 [SMILES: c1ccc(-c2ccc3c(-c4nc(-c5ccccc5)nc(-c5cccc6sc7ccccc7c56)n4)cc4nc(-c5ccccc5)oc4c3c2)cc1 ; c1ccc(-c2ccc3c(-c4nc(-c5ccc6ccccc6c5)nc(-c5cccc6[se]c7ccccc7c56)n4)cc4nc(-c5ccccc5)oc4c3c2)cc1 ; c1ccc(-c2ccc3c(c2)c(-c2nc(-c4ccc5ccccc5c4)nc(-c4cccc5sc6ccccc6c45)n2)cc2nc(-c4ccccc4)oc23)cc1]

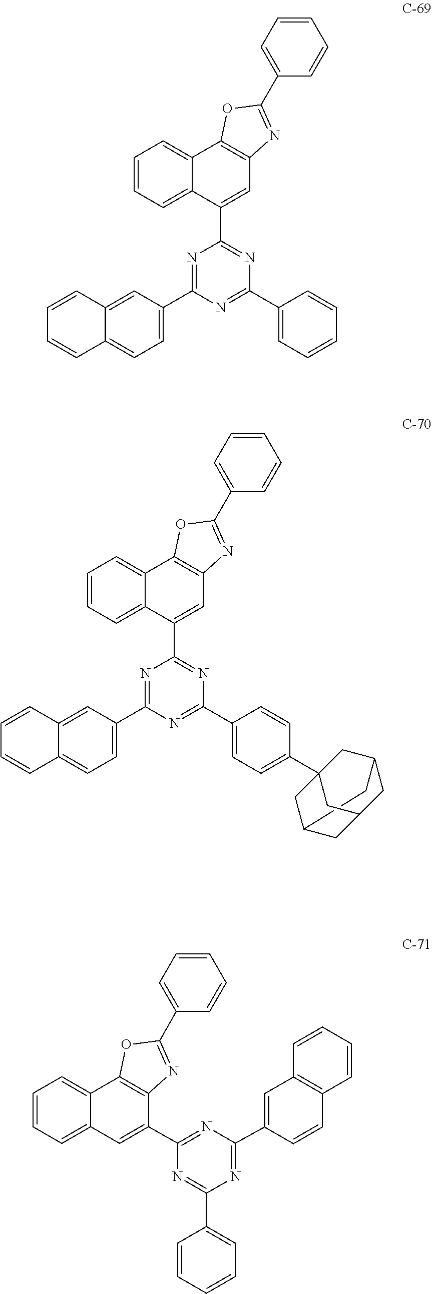 [SMILES: c1ccc(-c2nc(-c3ccc4ccccc4c3)nc(-c3cc4nc(-c5ccccc5)oc4c4ccccc34)n2)cc1 ; c1ccc(-c2nc3cc(-c4nc(-c5ccc(C67CC8CC(CC(C8)C6)C7)cc5)nc(-c5ccc6ccccc6c5)n4)c4ccccc4c3o2)cc1 ; c1ccc(-c2nc(-c3ccc4ccccc4c3)nc(-c3cc4ccccc4c4oc(-c5ccccc5)nc34)n2)cc1]

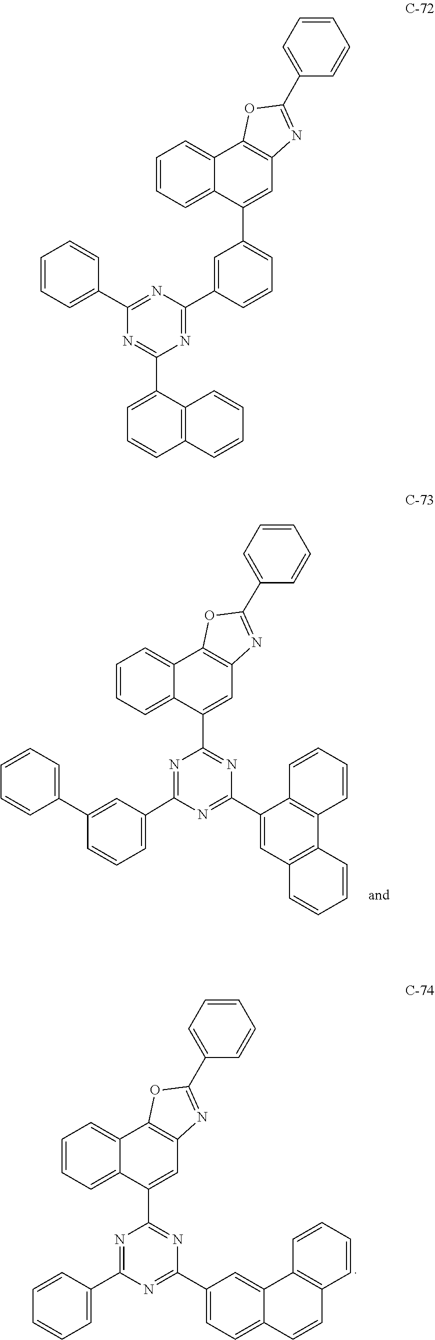 [SMILES: c1ccc(-c2nc(-c3cccc(-c4cc5nc(-c6ccccc6)oc5c5ccccc45)c3)nc(-c3cccc4ccccc34)n2)cc1 ; c1ccc(-c2cccc(-c3nc(-c4cc5ccccc5c5ccccc45)nc(-c4cc5nc(-c6ccccc6)oc5c5ccccc45)n3)c2)cc1 ; c1ccc(-c2nc(-c3ccc4ccc5ccccc5c4c3)nc(-c3cc4nc(-c5ccccc5)oc4c4ccccc34)n2)cc1]

10. An organic electroluminescent device comprising the compound according to claim 8.
